SEQUENCE LISTING (68 sequences)

SEQ ID NO: 1 - RNA, 282 nt, synthetic construct
  source 1..282: mol_type = other RNA
gatgggcggcgcataagagaagcccagaccaattacctacccaaaatcgagaaagttcacgttgacatcgaggaagacagcccattcctcagagctttgcagcggagcttccggcagtttgaggtagaagccaaggaggtgactgataatcaccatcctattgccagagcgttttcgcatctggcttcaaaactgatcgaaacggaggtggacccatccgacacgatccttgacattggaagtgcgcccgcccgcagaaagtattctactagtgccaccatg

SEQ ID NO: 2 - DNA, 64 bp, synthetic construct
  source 1..64: mol_type = other DNA; note = Pre-miR-lacZ
tgctgaaatcgctgatttgtgtagtcgttttggccactgactgacgactacacatcagcgattt

SEQ ID NO: 3 - DNA, 64 bp, synthetic construct
  source 1..64: mol_type = other DNA; note = Pre-miR-neco
tgctgaaatgtactgcgcgtggagacgttttggccactgactgacgtctccacgcagtacattt

SEQ ID NO: 4 - DNA, 64 bp, synthetic construct
  source 1..64: mol_type = other DNA; note = Pre-miR-luc1
tgctgagcccatatcgtttcatagctgttttggccactgactgacagctatgacgatatgggct

SEQ ID NO: 5 - DNA, 64 bp, synthetic construct
  source 1..64: mol_type = other DNA; note = Pre-miR-luc2
tgctgatacctggcagatggaacctcgttttggccactgactgacgaggttccctgccaggtat

SEQ ID NO: 6 - DNA, 60 bp, synthetic construct
  source 1..60: mol_type = other DNA; note = Pre-miR-p53-1
tgctgtccacacgcaaatttccttccgttttggccactgacggaaggaattgcgtgtgga

SEQ ID NO: 7 - DNA, 60 bp, synthetic construct
  source 1..60: mol_type = other DNA; note = Pre-miR-p53-2
tgctgagtagattaccactggagtctgttttggccactgacagactccaggtaatctact

SEQ ID NO: 8 - DNA, 60 bp, synthetic construct
  source 1..60: mol_type = other DNA; note = Pre-miR-p53-3
tgctgcaaacacgcacctcaaagctggttttggccactgaccagctttggtgcgtgtttg

SEQ ID NO: 9 - DNA, 64 bp, synthetic construct
  source 1..64: mol_type = other DNA; note = *Scrambled control
atggatcccaccaagttgagttacgtactaagttctgagtcgtcgcccgcgtttattgatatgt

SEQ ID NO: 10 - DNA, 64 bp, synthetic construct
  source 1..64: mol_type = other DNA; note = Pre-miR-VIP1
tgctgtctataatccctacaccacctgttttggccactgactgacaggtggtggggattataga

SEQ ID NO: 11 - DNA, 64 bp, synthetic construct
  source 1..64: mol_type = other DNA; note = Pre-miR-VIP2
tgctgtaccaatccagcttcagatcagttttggccactgactgactgatctgactggattggta

SEQ ID NO: 12 - DNA, 42 bp, synthetic construct
  source 1..42: mol_type = other DNA; note = VEEV-5CSE-emGFP-fw
ccgcagaaagtattctactagtccaccatggtgagcaagggc

SEQ ID NO: 13 - DNA, 44 bp, synthetic construct
  source 1..44: mol_type = other DNA; note = VEEV-SGP-emGFP-fw
atagtctagtccgccaagactagtccaccatggtgagcaagggc

SEQ ID NO: 14 - DNA, 46 bp, synthetic construct
  source 1..46: mol_type = other DNA; note = miR-3CSE-VEEV-rv
aattgctgctgtattcatgcggccgctgggccatttgttccatgtg

SEQ ID NO: 15 - DNA, 79 bp, synthetic construct
  source 1..79: mol_type = other DNA; note = miR-scrambled-lacZ-rv1
tatcaataaacgcgggcgacgactcagaacttagtacgtaactcaacttggtgggatccattacagccttcagcaagcc

SEQ ID NO: 16 - DNA, 96 bp, synthetic construct
  source 1..96: mol_type = other DNA; note = miR-scrambled-VEEV-rv2
aattgctgctgtattcatgcggccgctgggccatttgttccatgtgagtgctagtaacaggccttgtgtcctgacatatcaataaacgcgggcgac

SEQ ID NO: 17 - DNA, 43 bp, synthetic construct
  source 1..43: mol_type = other DNA; note = SINV-SGP-emGFP-fw
actacaacaccaccacccgatcgccaccatggtgagcaagggc

SEQ ID NO: 18 - DNA, 43 bp, synthetic construct
  source 1..43: mol_type = other DNA; note = SINV-3CSE-emGFP-fw
gctcgtagaacgataaacccgggccaccatggtgagcaagggc

SEQ ID NO: 19 - DNA, 46 bp, synthetic construct
  source 1..46: mol_type = other DNA; note = miR-3CSE-SINV-rv
gcagacgctgcgtggcggccgcgatctgggccatttgttccatgtg

SEQ ID NO: 20 - DNA, 96 bp, synthetic construct
  source 1..96: mol_type = other DNA; note = miR-scrambled-SINV-rv2
gcagacgctgcgtggcggccgcgatctgggccatttgttccatgtgagtgctagtaacaggccttgtgtcctgacatatcaataaacgcgggcgac

SEQ ID NO: 21 - DNA, 93 bp, synthetic construct
  source 1..93: mol_type = other DNA; note = VEE-pA60-rv
ttttttttttttttttttttttttttttttttttttttttttttttttttttttttttttgaaatattaaaaacaaaatccgattcggaaaag

SEQ ID NO: 22 - DNA, 27 bp, synthetic construct
  source 1..27: mol_type = other DNA; note = VEE-T7-5UTR-ATGG
taattaatacgactcactatagatggg

SEQ ID NO: 23 - DNA, 21 bp, synthetic construct
  source 1..21: mol_type = other DNA; note = Human HPRT sense
tgacactggcaaaacaatgca

SEQ ID NO: 24 - DNA, 21 bp, synthetic construct
  source 1..21: mol_type = other DNA; note = Human HPRT antisense
ggtccttttcaccagcaagct

SEQ ID NO: 25 - DNA, 20 bp, synthetic construct
  source 1..20: mol_type = other DNA; note = *Hamster beta-actin sense
cctgtatgccaacacagtgc

SEQ ID NO: 26 - DNA, 20 bp, synthetic construct
  source 1..20: mol_type = other DNA; note = *Hamster beta-actin antisense
atactcctgcttgctgatcc

SEQ ID NO: 27 - DNA, 20 bp, synthetic construct
  source 1..20: mol_type = other DNA; note = Bacterial lacZ gene sense
gtacgtcttcccgagcgaaa

SEQ ID NO: 28 - DNA, 20 bp, synthetic construct
  source 1..20: mol_type = other DNA; note = Bacterial lacZ gene antisense
ctgttgactgtagcggctga

SEQ ID NO: 29 - DNA, 20 bp, synthetic construct
  source 1..20: mol_type = other DNA; note = Human p53 gene sense
acactcgcttctgaatcatc

SEQ ID NO: 30 - DNA, 20 bp, synthetic construct
  source 1..20: mol_type = other DNA; note = Human p53 gene antisense
gagaccattcataagcaacg

SEQ ID NO: 31 - DNA, 23 bp, synthetic construct
  source 1..23: mol_type = other DNA; note = Human RSAD2 gene sense
tgccacaatgtgggtgcttacac

SEQ ID NO: 32 - DNA, 20 bp, synthetic construct
  source 1..20: mol_type = other DNA; note = Human RSAD2 gene antisense
ctcaaggggcagcacaaagg

SEQ ID NO: 33 - DNA, 21 bp, synthetic construct
  source 1..21: mol_type = other DNA; note = Human TGFbetaR2 sense
tgagtccttcaagcagaccga

SEQ ID NO: 34 - DNA, 21 bp, synthetic construct
  source 1..21: mol_type = other DNA; note = Human TGFbetaR2 antisense
acacaccatctggatgccctg

SEQ ID NO: 35 - DNA, 20 bp, synthetic construct
  source 1..20: mol_type = other DNA; note = Human DAZAP2 sense
cgaacaggaagaggacgaaa

SEQ ID NO: 36 - DNA, 20 bp, synthetic construct
  source 1..20: mol_type = other DNA; note = Human DAZAP2 antisense
cagggtaggttggctgtgtt

SEQ ID NO: 37 - DNA, 29 bp, synthetic construct
  source 1..29: mol_type = other DNA; note = murine miR-155 backbone part one
ctggaggcttgctgaaaggctgtatgctg

SEQ ID NO: 38 - DNA, 17 bp, synthetic construct
  source 1..17: mol_type = other DNA; note = murine miR-155 backbone part two
ttttggcctctgactga

SEQ ID NO: 39 - DNA, 43 bp, synthetic construct
  source 1..43: mol_type = other DNA; note = murine miR-155 backbone part three
ggacacaaggcctgttactagcactcacatggaacaaatggcc

SEQ ID NO: 40 - RNA, 87 nt, synthetic construct
  source 1..87: mol_type = other RNA; note = pre miR-p53-2
aggctgtatgctgagtagattaccactggagtctgttttggccactgactgacagactccaggtaatctactcaggacacaaggcct

SEQ ID NO: 41 - RNA, 105 nt, synthetic construct
  source 1..105: mol_type = other RNA
gctaagcacttcgtggccgtcgatcgtttaaagggaggtagtgagtcgaccagtggatcctggaggcttgctgagttactagcactcacatggaacaaatggccc

SEQ ID NO: 42 - DNA, 110 bp, synthetic construct
  source 1..110: mol_type = other DNA; note = polyA tail (A30LinkerA70)
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 43 - DNA, 317 bp, synthetic construct
  source 1..317: mol_type = other DNA; note = FI-element
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 44 - DNA, 249 bp, synthetic construct
  source 1..249: mol_type = other DNA; note = sec/MITD
atgagagtgatggcccccagaaccctgatcctgctgctgtctggcgccctggccctgacagagacatgggccggaagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaa

SEQ ID NO: 45 - DNA, 195 bp, synthetic construct
  source 1..195: mol_type = other DNA; note = P2P16 helper epitope
aagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 46 - DNA, 24 bp, synthetic construct
  source 1..24: mol_type = other DNA; note = subgenomic promoter
ctctctacggctaacctgaatgga

SEQ ID NO: 47 - DNA, 187 bp, synthetic construct
  source 1..187: mol_type = other DNA; note = 5' UTR pST1-OPAL+ 51nt - CSE
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttc

SEQ ID NO: 48 - DNA, 187 bp, synthetic construct; identical to SEQ ID NO: 47
  source 1..187: mol_type = other DNA; note = 5' UTR pVEE + 51nt - CSE

SEQ ID NO: 49 - DNA, 63 bp, synthetic construct
  source 1..63: mol_type = other DNA; note = 3' UTR pST1-OPAL
ggagcttaattcgacgaataattggatttttattttattttgcaattggtttttaatatttcc

SEQ ID NO: 50 - DNA, 121 bp, synthetic construct
  source 1..121: mol_type = other DNA; note = 3' UTR pVEE
tgaatacagcagcaattggcaagctgcttacatagaactcgcggcgattggcatgccgccttaaaatttttattttattttttcttttcttttccgaatcggattttgtttttaatatttc

SEQ ID NO: 51 - DNA, 152 bp, Homo sapiens
  source 1..152: mol_type = other DNA; note = human beta globin 3' UTR
agctcgctttcttgctgtccaatttctattaaaggttcctttgttccctattgttccctaagtccaactactaaactgggggatattatgaagggccttgagcatctggattctgcctaataaaaaacatttattttcattgctgcgtcgag

SEQ ID NO: 52 - DNA, 52 bp, synthetic construct
  source 1..52: mol_type = other DNA; note = human alpha globin 5' UTR
gggcgaactagtattcttctggtccccacagactcagagagaacccgccacc

SEQ ID NO: 53 - RNA, 21 nt, synthetic construct
  source 1..21: mol_type = other RNA
aaatgtactgcgcgtggagac

SEQ ID NO: 54 - RNA, 21 nt, synthetic construct
  source 1..21: mol_type = other RNA
aaatcgctgatttgtgtagtc

SEQ ID NO: 55 - RNA, 21 nt, synthetic construct
  source 1..21: mol_type = other RNA
agcccatatcgtttcatagct

SEQ ID NO: 56 - RNA, 21 nt, synthetic construct
  source 1..21: mol_type = other RNA
atacctggcagatggaacctc

SEQ ID NO: 57 - RNA, 21 nt, synthetic construct
  source 1..21: mol_type = other RNA
tccacacgcaaatttccttcc

SEQ ID NO: 58 - RNA, 21 nt, synthetic construct
  source 1..21: mol_type = other RNA
agtagattaccactggagtct

SEQ ID NO: 59 - RNA, 21 nt, synthetic construct
  source 1..21: mol_type = other RNA
caaacacgcacctcaaagctg

SEQ ID NO: 60 - DNA, 19 bp, synthetic construct
  source 1..19: mol_type = other DNA
ctggaccaagtccttgaac

SEQ ID NO: 61 - DNA, 18 bp, synthetic construct
  source 1..18: mol_type = other DNA
cgctcagaccttcatacg

SEQ ID NO: 62 - RNA, 23 nt, synthetic construct
  source 1..23: mol_type = other RNA
taagtgcttccatgttttggtga

SEQ ID NO: 63 - RNA, 23 nt, synthetic construct
  source 1..23: mol_type = other RNA
taagtgcttccatgttttagtag

SEQ ID NO: 64 - RNA, 23 nt, synthetic construct
  source 1..23: mol_type = other RNA
taagtgcttccatgtttcagtgg

SEQ ID NO: 65 - RNA, 23 nt, synthetic construct
  source 1..23: mol_type = other RNA
taagtgcttccatgtttgagtgt

SEQ ID NO: 66 - RNA, 22 nt, synthetic construct
  source 1..22: mol_type = other RNA
aattgcactttagcaatggtga

SEQ ID NO: 67 - RNA, 87 nt, synthetic construct
  source 1..87: mol_type = other RNA
aggctgtatgctgaaatcgctgatttgtgtagtcgttttggccactgactgacgactacacatgagcgatttcaggacacaaggcct

SEQ ID NO: 68 - RNA, 87 nt, synthetic construct
  source 1..87: mol_type = other RNA
aggctgtaatggatcccaccaagttgagttacgtactaagttctgagtcgtcgcccgcgtttattgatatgtcaggacacaaggcct

(19) United States
(12) Patent Application Publication
(10) Pub. No.: US 2026/0366030 A1
(43) Pub. Date: Nov. 19, 2026

(54) SYSTEMS AND COMPOSITIONS COMPRISING TRANS-AMPLIFYING RNA VECTORS WITH MIRNA

(71) Applicants: BioNTech SE, Mainz (DE); TRON-Translationale Onkologie an der Universitäsmedizin der Johannes Gutenberg-Universität Mainz, Mainz (DE)

(72) Inventors: Tim BEISSERT, Mainz (DE); Mario PERKOVIC, Mainz (DE); Ugur SAHIN, Mainz (DE); Aysegül YILDIZ, Mainz (DE)

(21) Appl. No.: 19/111,586

(22) PCT Filed: Sep. 15, 2023
(86) PCT No.: PCT/EP2023/075427

§ 371 Date: Mar. 13, 2025

(30) Foreign Application Priority Data
Sep. 15, 2022 (WO) PCT/EP2022/075653
Aug. 24, 2023 (WO) PCT/EP2023/073256

(51) Int. Cl. C12N 15/63; A61K 31/7105; A61K 31/7115; A61P 35/00; C12N 9/12

(52) U.S. Cl. CPC C12N 15/63; A61K 31/7105; A61K 31/7115; A61P 35/00; C12N 9/127; C12Y 207/07048; C12N 2310/141; C12N 2310/317; C12N 2310/335; C12N 2770/36122; C12N 2830/50

(57) ABSTRACT

The present invention embraces systems, kits and compositions comprising two RNA molecules, wherein the first RNA molecule comprises an open reading frame encoding a functional RNA-dependent RNA polymerase (replicase) and wherein the second RNA molecule is a replicable RNA molecule comprising at least one miRNA sequence, which miRNA sequence is capable of being excised from the second replicable RNA when present in a cell, and is capable of regulating gene expression in a cell, and which replicable RNA molecule is capable of being replicated in trans by the replicase encoded by the first RNA molecule. The present invention further embraces methods for the treatment or prevention of cancer or an infection or other diseases and disorders with such systems and compositions as well as the use of such systems and compositions in such treatment and prevention methods.

### TECHNICAL FIELD

The present invention embraces systems, kits and compositions comprising two RNA molecules, wherein the first RNA molecule comprises an open reading frame encoding a functional RNA-dependent RNA polymerase (replicase) and wherein the second RNA molecule is a replicable RNA molecule comprising at least one miRNA sequence, which miRNA sequence is capable of being excised from the second replicable RNA when present in a cell, and is capable of regulating gene expression in a cell, and which replicable RNA molecule is capable of being replicated in trans by the replicase encoded by the first RNA molecule. The present invention further embraces methods for the treatment or prevention of cancer or an infection or other diseases and disorders with such systems and compositions as well as the use of such systems and compositions in such treatment and prevention methods.

### BACKGROUND

Alphaviruses belong to the virus family Togaviridae that are enveloped positive-stranded RNA viruses. Alphaviruses can infect insects, fish and mammals, such as domesticated animals and humans. Alphaviruses replicate in the cytoplasm of infected cells (for review of the alphaviral life cycle see José et al., 2009, Future Microbiol. 4:837-856). The genomic RNA of alphaviruses is 5′capped, 3′polyadenylated and between 11 and 12 kilo nucleotides long (J. H. Strauss and E. G. Strauss, Microbiol. Rev., vol. 58, no. 3, pp. 491-562, 1994; J. Y.-S. Leung, M. M.-L. Ng, and J. J. H. Chu, Adv. Virol., vol. 2011, p. 249640, 2011). It possesses two open-reading frames (ORFs). The first ORF encodes a large polyprotein, nsP1234, which builds replication complexes necessary for RNA transcription, modification and replication. The second ORF, which is under the control of the subgenomic promotor (SGP), encodes the structural proteins necessary to form the virus particle (C. M. Rice and J. H. Strauss Proc. Natl. Acad. Sci. U.S.A, vol. 78, no. 4, pp. 2062-6, April 1981). This bicistronic mRNA is flanked by conserved sequence elements (CSE), which form RNA structures, required for subgenomic transcription and replication (J. H. Strauss and E. G. Strauss, Microbiol. Rev., vol. 58, no. 3, pp. 491-562, 1994).

An infection with an alphavirus leads to the direct translation of the viral non-structural proteins from the genomic RNA, while the structural proteins are translatable from a subgenomic transcript (Gould et al., Antiviral Res. 87:111-124, 2010). Early in infection, newly translated nsP1234 is autoproteolytically cleaved into the short-lived alphaviral polyprotein intermediate nsP123 and the non-structural protein 4 (nsP4). nsP123 interact with nsP4 proteins, which form the core viral RNA-dependent RNA polymerases (M. K. Pietilä, K. Hellström, and T. Ahola, Virus Res., 2017). Anti-sense RNA synthesis of the (+) genomic RNA is induced, generating at least one complementary (−) genomic copy as template for positive-strand RNA synthesis. Right after the generation of anti-sense RNA templates, nsP123 is processed sequentially into nsP1 and nsP23, and the latter eventually to nsP2 and nsP3 by viral nsP2 proteases. Together with nsP4, they all form the stable replicase protein or replication complex (L. Carrasco, M. A. Sanz, and E. González-Almela, Viruses, vol. 10, no. 2, 2018). These replication complexes are then transcribing and amplifying positive-sense genomic and subgenomic RNAs (sgRNAs). At the late phase of infection, sgRNAs for the structural proteins are transcribed only, which are necessary for encapsidation of viral RNA, final assembly and virus release.

To generate an alphaviral-based self-amplifying RNA or saRNA vector, heterologous genes of interest (GOI) replace the structural genes within the genomic alphaviral RNA. The replicase polyprotein remains to enable augmented GOI expression resulting from very high numbers of newly synthesized saRNA copies. In this kind of system, virion formation and virus spreading is barred since the structural proteins are lacking (J. H. Aberle, S. W. Aberle, R. M. Kofler, and C. W. Mandl, J. Virol., vol. 79, no. 24, pp. 15107-13, December 2005). However, the RNA replication process of the saRNA is identical to the genome replication in an alphavirus infected cell. Furthermore, transient transfection with saRNA elicits a strong immune response since double-stranded RNA (dsRNA) replication intermediates activate the innate immune system. This equals an intrinsic, self-adjuvanting activity triggering and enhancing the immune response of the host (N. P. Restifo et al., Nat. Med., vol. 5, no. 7, pp. 823-827, July 1999; Perri et al., J. Virol., vol. 77, no. 19, pp. 10394-403, October 2003). This, alongside of being a vehicle to deliver antigens, makes a saRNA a suitable and attractive candidate as an RNA vaccine (A. J. Geall et al., Proc. Natl. Acad. Sci. U.S.A, vol. 109, no. 36, pp. 14604-9, 2012; J. B. Ulmer and A. J. Geall, Curr. Opin. Immunol., vol. 41, pp. 18-22, 2016).

Trans-amplifying or taRNA is a split-vector system comprising two alphaviral sequence-based RNA molecules. One is a capped, replication-incompetent in vitro transcribed (IVT) mRNA encoding the replicase polyprotein. The GOI-encoding IVT RNA is flanked by viral 5′CSE and 3′ CSE so that it is able to be replicated by the replicase protein in trans (called transreplicon (TR) and/or nano-transreplicon (NTR)) (J. O. Rayner, S. A. Dryga, and K. I. Kamrud, Reviews in Medical Virology, vol. 12, no. 5. pp. 279-296, 2002). Upon delivery of both RNA constructs into a cell, the mRNA templated viral replicase protein recognizes the 5′CSE and 3′CSE of the co-transferred TR/NTR and amplifies it in trans.

Gene-controlling systems based on small non-coding RNAs (sncRNAs) are universal in biology. Present in animals, plants and viruses, sncRNAs are <200-nucleotide (nt) in length, endogenous or exogenous, single- or double-stranded RNA molecules (R. W. Carthew and E. J. Sontheimer, Cell, vol. 136, no. 4, pp. 642-55, February 2009). When associated with specialized proteins they help to suppress the expression of invading genes (e.g. of viral origin), or they regulate the expression of the cell's own transcriptome. These mechanisms are generally summarized as RNA interference (RNAi) (A. Fire, S. Xu, M. K. Montgomery, S. A. Kostas, S. E. Driver, and C. C. Mello, Nature, vol. 391, no. 6669, pp. 806-811, 1998; R. C. Wilson and J. A. Doudna, Annu. Rev. Biophys., vol. 42, no. 1, pp. 217-239, 2013.20). In bacteria, clustered regularly interspaced short palindromic repeat RNAs are a crucial component of the endogenous immune defense system, defending invading foreign nuclei acid (L. A. Marraffini and E. J. Sontheimer, Nat. Rev. Genet., vol. 11, no. 3, pp. 181-90, March 2010). In invertebrates and plants, small interfering RNAs (siRNAs) make up the host anti-viral defense system. siRNAs are generated from endogenous, exogenous or viral double-stranded RNAs, and induce post-transcriptional silencing (PTS) of viral transcripts and transposons (S.-W. Ding, Nat. Rev. Immunol., vol. 10, no. 9, pp. 632-44, September 2010). On the contrary, microRNAs (miRNAs) represent a unique class of sncRNA conserved in eukaryotes and responsible for PTS of endogenous mRNAs (M. Ghildiyal and P. D. Zamore, Nature Reviews Genetics, vol. 10, no. 2. pp. 94-108, 2009.24). Currently 1984 pre-miRNA sequences (as of 14.07.2022 taken from data base miRBase version 22.1) are known in humans. Each miRNA possibly targets several hundred genes and all together affect at least 50% of all human genes [(R. C. Friedman, K. K. H. Farh, C. B. Burge, and D. P. Bartel, Genome Res., vol. 19, no. 1, pp. 92-105, 2009). A single mRNA can be regulated by several miRNAs (D. P. Bartel, Cell, vol. 131, no. 4, pp. 11-29, 2007). Since so many protein-coding transcripts are regulated by miRNAs, they play a vital role in nearly all developmental and pathological processes in animals. Hence, defects or dysregulation of miRNAs is associated with many diseases like neurological disorders, cancer and cardiovascular diseases (M. V. Lorio and C. M. Croce, EMBO molecular medicine, vol. 4,3, pp. 143-59, 2012; C. Urbich, A. Kuehbacher, and S. Dimmeler, Cardiovascular Research, vol. 79, no. 4. pp. 581-588, 2008).

MiRNAs act directly on their target genes in a sequence-dependent manner (E. Huntzinger and E. Izaurralde, Nature Reviews Genetics, vol. 12, no. 2. pp. 99-110, 2011). In detail, a mature miRNA associates with Argonaute (AGO) proteins, the effectors of gene silencing, to form the so-called RNA-induced silencing complex (RISC). RISC is a ribonucleoprotein complex, which mediates post-transcriptional silencing (PTS) under the guidance of the miRNA.

The first 2-7 nucleotides at the 5′ end of miRNAs is the seed sequence. This section forms base pairs with the 3′ UTR of the target mRNA and depending on the number of base pair matches, AGO, the active part of RISC, induces cleavage, destabilization or translational inhibition of the target mRNA (D. P. Bartel, Cell, vol. 136, no. 2. pp. 215-233, 2009). Most commonly, a low level of PTS (~20%) is the consequence since most miRNA target sites only have partial complementarity to their target mRNA (D. Baek, J. Villén, C. Shin, F. D. Camargo, S. P. Gygi, and D. P. Bartel, Nature, vol. 455, no. 7209, pp. 64-71, September 2008; H. Seitz, Curr. Biol., vol. 19, no. 10, pp. 870-873, May 2009).

Since its discovery, RNAi mechanisms have continuously been exploited for basic and applied research, or genome engineering. Its superb potential when performing loss-of-function studies in animals has inspired the development of RNAi-based therapies to fight different genetic and viral diseases such as Huntington's disease or viral hepatitis (S. Aguiar, B. van der Gaag, and F. A. B. Cortese, Translational Neurodegeneration, vol. 6, no. 1. 2017; D. Castanotto and J. J. Rossi, Nature, vol. 457, no. 7228. pp. 426-433, 2009).

For sequence-dependent cleavage and reduction of protein coding transcripts, siRNAs and shRNAs are commonly used as RNAi mediators. shRNAs are synthetic short-hairpin RNAs that mimic miRNA precursors. Also, engineered miRNAs, exogenous miRNAs, are used to achieve gene control. RNAi mediators are generally expressed from plasmids or viral DNA-expression vectors, either transiently or stably. For stable silencing of gene expression, excessive efforts have been made on the construction and delivery of miRNA expression cassettes with viral vectors. Four popular and well-studied viral vector systems based on viruses are commonly used to facilitate high level transgene and miRNA expression: adenovirus and adenovirus-associated virus, retrovirus, and the subclass lentivirus. The different viral vector systems have their own advantages and disadvantages. Main drawbacks of adenovirus-based vectors are the requirement of repeated administration and their relatively high immunogenicity. The use of adeno-associated virus-based vectors requires helper virus for replication. Furthermore, this vector system has an overall limited insert capacity (max. 3-5 kb). The well-known main concern of retro- and lentiviral vectors is their risk of insertional mutagenesis (for details see review by E. Herrera-Carrillo, Y. P. Liu, and B. Berkhout, Hum. Gene Ther. Methods, vol. 28, no. 4, 2017).

Thus, there remains an urgent need for systems and compositions providing for high copy numbers of miRNA precursors. Furthermore, there remains a need for efficient systems in which a miRNA could easily be co-transferred together with a protein-coding gene. The present invention fulfills such need.

### SUMMARY

The present invention generally relates to systems comprising two RNA molecules, wherein the first RNA molecule comprises an open reading frame encoding a functional RNA-dependent RNA polymerase (replicase) and wherein the second RNA molecule is a replicable RNA molecule comprising at least one miRNA sequence, which miRNA sequence is capable of being excised from the second replicable RNA when present in a cell, and is capable of regulating gene expression in a cell, and which replicable RNA molecule can be replicated in trans by the replicase encoded by the first RNA molecule.

Without being bound by theory, in some embodiments the second RNA molecule is similar to a primary miRNA (pri-miRNA) from which a miRNA is excised by enzymes present inside a cell, e.g., Drosha and/or Dicer. In accordance with the invention described herein, the second RNA molecule is processed inside the cell to excise the miRNA sequence out of the larger sequence of the second RNA molecule to provide a functional miRNA, which functional miRNA can form an RNA induced silencing complex (RISC) together with proteins from the host cell. Also, the second RNA molecule can be processed inside the cell to excise first a pre-miRNA molecule from the pri-miRNA sequence, which then can be further processed in the cell to obtain a functional miRNA. In some embodiments, a difference between a naturally occurring pri-miRNA and the second RNA molecule of the present invention is that the second RNA molecule comprises, in addition to the miRNA sequence, sequences needed for the replication of the second RNA molecule by the replicase encoded by the first RNA molecule. The second RNA molecule, similar to the pri-miRNA sequence, comprises sequences needed for excising the miRNA out of the second RNA molecule. Such excising can take place inside the cell using cellular mechanisms, e.g., using enzymes present inside the cell, such as RNA-cleaving enzymes, ribonucleases, ribozymes, etc. to excise the functional miRNA sequence.

In an embodiment, the second RNA molecule comprises at least one pre-miRNA sequence. The miRNA sequence in this embodiment is flanked by further sequences, which together with the miRNA sequence form a pre-miRNA sequence. The excision from the second RNA molecule typically happens in a cell capable of excising the miRNA sequence from the second RNA molecule, e.g. a cell which expresses Drosha and Dicer.

The present invention is also partially based, without being bound by theory, on the observation that a miRNA can be introduced into a cell without the need to introduce the pri-miRNA into the nucleus where the pri-miRNA is normally processed. The present invention is also based on the observation that it is beneficial to include the miRNA sequence on a replicable RNA in order to enhance the efficiency of the miRNA in regulating gene expression, e.g., by interfering with the translation of a mRNA molecule to which the miRNA binds.

In one embodiment, the first and/or second RNA molecule, preferably the second RNA molecule, further comprises at least one open reading frame (ORF) encoding a protein of interest. The inventors have surprisingly found that a miRNA sequence can be combined with a coding sequence of a protein of interest on the same replicable RNA such that the protein of interest and the miRNA can be provided to a subject at the same time, as well as in the same cell. For example, the protein of interest can be a de-differentiation factor and the miRNA can inhibit the expression of a gene responsible for differentiation or be a stem cell-specific miRNA (i.e., the miRNA is preferentially expressed in a stem cell compared to a differentiated cell). In another example, the protein of interest can be a tumor antigen and the miRNA can inhibit the expression of an oncogene expressed in the tumor.

In an embodiment, the miRNA can be a stem cell-specific miRNA (i.e., the miRNA is preferentially expressed in a stem cell compared to a differentiated cell) and the protein of interest can be factor for inducing pluripotency, e.g., OCT4. In an embodiment, the miRNA sequence targets an mRNA encoding a protein that is overexpressed in a cancer cell and the open reading frame encodes a protein useful in the treatment of said cancer.

Described herein are systems comprising two RNA molecules, wherein the first RNA molecule comprises an open reading frame encoding a functional RNA-dependent RNA polymerase (replicase) and wherein the second RNA molecule is a replicable RNA molecule comprising at least one non-coding RNA sequence which is capable of being excised from the second replicable RNA molecule when present in a cell and is capable of regulating gene expression in a cell, and which replicable RNA molecule can be replicated in trans by the replicase encoded by the first RNA molecule. In an embodiment, the second RNA molecule further comprises at least one open reading frame (ORF) encoding a protein of interest, as described herein. Each non-coding RNA sequence comprised in the second RNA molecule may be 10-500 nucleotides in length, optionally 10-400, 10-300, 10-200, 10-100, 10-50, 20-400, 20-300, 20-200, 20-100, 20-50, 10-40, 10-30, 20-40, or 20-30 nucleotides in length, optionally 10-100, preferably 10-50 nucleotides in length. Exemplary non-coding RNA sequences include miRNAs, shRNAS, siRNAs, and antisense molecules, but the skilled person will be aware of other non-coding RNA sequences capable of regulating gene expression in a cell which may be incorporated into the second replicable RNA molecule. In an embodiment, the second RNA molecule can be an mRNA. In an embodiment, the second RNA molecule can be a replicable RNA molecule and an mRNA.

In an embodiment, the first RNA molecule can be a replicable RNA molecule that can be replicated by its encoded replicase. In an embodiment, the first RNA molecule is not a replicable RNA molecule. In an embodiment, the first RNA molecule can be an mRNA. In an embodiment, the first RNA molecule can be an mRNA and the second RNA molecule can be an mRNA. In an embodiment, the first RNA molecule is an mRNA and is not a replicable RNA molecule, and the second RNA molecule is an mRNA and is a replicable RNA molecule. In an embodiment, the first RNA molecule is an mRNA and is a replicable RNA molecule, and the second RNA molecule is an mRNA and is a replicable RNA molecule.

In an embodiment, the replicase is derived from the functional non-structural protein from a self-replicating virus. In an embodiment, the self-replicating virus is a self-replicating single-stranded RNA virus. In an embodiment, the self-replicating virus is a positive-sense, single-stranded RNA virus (e.g., alphavirus, flavivirus, etc.). In an embodiment, the self-replicating virus is an alphavirus, preferably selected from the group consisting of Venezuelan equine encephalitis virus, Eastern equine encephalitis virus, Western equine encephalitis virus, Chikungunya virus, Semliki Forest virus, Sindbis virus, Barmah Forest virus, Middelburg virus and Ndumu virus. Preferably, the alphavirus is Venezuelan equine encephalitis virus or Semliki Forest virus.

In an embodiment, the second RNA molecule can comprise at least two, at least three, at least four, at least five, or at least ten miRNA sequences, preferably at least 5 miRNA sequences. In an embodiment, the second RNA molecule can comprise 1-20, 1-10, 1-8, 1-5, 1-4, 1-3 or 1-2 miRNA sequences, optionally 1-10, preferably 2-8 miRNA sequences. In an embodiment, the second RNA molecule can comprise 1-20, 1-10, 1-8, 1-5, 1-4, 1-3 or 1-2 different miRNA sequences, optionally 1-10, preferably 2-8 different miRNA sequences. In an embodiment, the second RNA molecule can comprise 1-20, 1-10, 1-8, 1-5, 1-4, 1-3 or 1-2 copies of the same miRNA sequence, optionally 1-10, preferably 2-8 copies of the same miRNA sequence.

In an embodiment, the sequence of at least one miRNA can differ in its sequence from at least one other miRNA, preferably wherein the sequence of each miRNA differs from the other. In an embodiment, the sequences of the miRNAs can be the same sequence.

In an embodiment, the miRNA targets an mRNA and can affect the translation of the mRNA such that the expression of the gene encoding the mRNA can be regulated. For example, the miRNA binds the mRNA such that the mRNA cannot be translated. In an embodiment, the same or different miRNAs can target the same mRNA. In an embodiment, the different miRNAs target different mRNAs. In an embodiment, the different miRNAs target 1, 2, 3, 4, 5, 6, 7, 8, 9, 10 or more different mRNAs, preferably 1-5 different mRNAs. In an embodiment, each of the miRNA sequences comprised in the second RNA molecule can target a different mRNA. In an embodiment, the different miRNAs target can different sites on the same mRNA, or wherein the different miRNAs target different sites on two or more mRNAs.

In an embodiment, the miRNA sequence can be a naturally occurring miRNA sequence, preferably a human miRNA sequence. In an embodiment, the miRNA sequence can be an artificial miRNA sequence. In an embodiment, the miRNA can be a non-viral miRNA. In an embodiment, the miRNA can be a stem cell specific miRNA.

In an embodiment, the miRNA can supress the innate immune response in a cell, e.g., targets the mRNA of a cytokine that contributes to the innate immune response, such as an interleukin.

In an embodiment, the target of the miRNA can be an mRNA relevant for the onset or progression of a disease, preferably an mRNA of an oncogene, mutated tumor suppressor gene or of a viral, bacterial or fungal gene. In an embodiment, the target of the miRNA can be a mutated (non-functional) tumor suppressor gene. For example, the mutated tumor suppressor gene is mutated TP53.

In an embodiment, the target of the miRNA can be an interferon stimulated gene, preferably RSAD2 (viperin). These genes can be upregulated upon infection with an alphavirus, which leads to inhibition of the translation machinery. In an embodiment, the target of the miRNA can be retinoic acid-inducible gene I (RIG-I). In an embodiment, the target of the miRNA can be the Eukaryotic Translation Initiation Factor 2 Alpha Kinase 2 (EIF2AK2) gene encoding protein kinase R (PKR). Not meant to be limiting, but the targeting of RIG-I and/or PKR has the advantage of helping to suppress innate immunity induced by transfection and results in the inhibition of the translation machinery in a cell.

In an embodiment, the target of the miRNA can be DAZ-associated protein 2 (DAZAP2) and/or TGF beta receptor 2 (TGFβR2).

In an embodiment, the sequence of the miRNA can be flanked 5′ and/or 3′ by flanking and loop sequences from a naturally occurring miRNA, preferably from murine miR-155, which flanking and loop sequences are, as is known in the miRNA art, required for excising the miRNA sequence from the larger sequence of the second RNA molecule. In this embodiment, the miRNA is preferably an artificial miRNA, in particular a miRNA that is designed to bind completely to its target mRNA. In an embodiment, the miRNA sequence can be at least one of miR-30 or miR-124. In an embodiment, the miRNA sequence can be at least one miRNA sequence of the miR-302/367 cluster. Preferably the miRNA sequence is all miRNA sequences of the miR-302/367 cluster. Preferably the miRNA sequence is the miR-302/367 cluster.

In an embodiment, the ORF can be flanked by a 5′ untranslated region (UTR) and/or a 3′ UTR.

Exemplary 5′ UTR sequences are depicted in SEQ ID NOs: 47, 48 and 52. In an embodiment, a 5′ UTR sequence useful in the RNA molecules described herein is one that is at least 75%, 80%, 85%, 90%, 95%, 98% or 99% homologous to SEQ ID NO: 47 or 48 or 52. Exemplary 3′ UTR sequences are depicted in SEQ ID Nos: 49, 50 and 51. In an embodiment, a 3′ UTR sequence useful in the RNA molecules described herein is one that is at least 75%, 80%, 85%, 90%, 95%, 98% or 99% homologous to SEQ ID NO: 49 or 50 or 51.

In an embodiment, the protein of interest can be a reporter protein, preferably GFP or a variant thereof. In an embodiment, the protein of interest can be a pharmaceutically active peptide or protein, pluripotency factor or differentiation factor, preferably a pluripotency factor. In an embodiment, the protein of interest can be an antigen or epitope, thereof, preferably a T cell epitope. In an embodiment, the protein of interest is a polyepitopic protein comprising more than one antigenic epitope. In an embodiment, the protein of interest comprises a signal sequence for extracellular expression and/or sequences which enhance expression or enhance presentation of the protein, e.g., epitope, on the surface of a cell, such as antigen presenting cells. In an embodiment, the protein of interest further comprises a MHC class I trafficking signal (MITD) and/or a HLA-II helper epitope, such as the P2P16 amino acid sequence derived from the tetanus toxoid of Clostridium tetanii. An exemplary MITD sequence is depicted in SEQ ID NO: 44. In an embodiment, a MITD sequence useful in the RNA molecules described herein is one that is at least 75%, 80%, 85%, 90%, 95%, 98% or 99% homologous to SEQ ID NO: 44. An exemplary P2P16 sequence is depicted in SEQ ID NO: 45. In an embodiment, a P2P16 sequence useful in the RNA molecules described herein is one that is at least 75%, 80%, 85%, 90%, 95%, 98% or 99% homologous to SEQ ID NO: 45.

In an embodiment, the antigen or epitope is a or is derived from a bacterial, viral, parasitical or fungal antigen. In an embodiment, the antigen or epitope is or is derived from a tumor antigen. Tumor antigens can be overexpressed in tumors or preferably are expressed only in tumors/tumor tissue.

In an embodiment, the protein of interest can be a Vaccinia virus immune evasion protein, such as E3 or B18.

In an embodiment, the sequence of the miRNA can be located anywhere in the second RNA molecule, as long as its insertion does not disrupt the translation or replication of the second RNA molecule. In an embodiment, the miRNA is not located in the 5′ or 3′ replication recognition sequences of the second RNA molecule. In an embodiment, the miRNA is not located within an ORF of the second RNA molecule. In an embodiment, the miRNA is not located in the poly(A) sequence of the second RNA molecule. In an embodiment, the sequence of the miRNA can be located in the 5′ untranslated region (UTR) or the 3′ untranslated region (UTR) of the ORF of the second RNA molecule. In an embodiment, the sequence of the miRNA can be located in the 3′ untranslated region (UTR) of the ORF of the second RNA molecule. In an embodiment, the 5′-end of the miRNA sequence can be connected to the ORF by a linker sequence. In an embodiment, the 3′-end of the miRNA sequence can be connected to the 3′ UTR of the second RNA molecule by a linker sequence. In an embodiment, each of the miRNA sequences can be connected by a linker sequence. In an embodiment, each linker sequence comprises at least one cleavage site which is capable of being cleaved when the second replicable RNA molecule is present in a cell. In an embodiment, the linker sequence can comprise 5 to 30 nucleotides.

In an embodiment, the first and/or second RNA molecule can be a modified RNA molecule or unmodified RNA molecule. Preferably the first and/or second RNA molecule is a modified RNA molecule.

In an embodiment, the first and/or second RNA molecule can be a modified RNA molecule comprising at least one modified uridine. Preferably at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the uridines in the RNA molecules are pseudouridine (ψ), N1-methyl-pseudouridine (m1ψ), or 5-methyl-uridine (m5U), preferably N1-methyl-pseudouridine (1mψ).

In an embodiment, the first and/or second RNA molecule can further comprise a 5′ cap, a 5′ regulatory region, a 5′ replication recognition sequence, a 3′ replication recognition sequence and/or a poly(A) sequence. In an embodiment, the first and/or second RNA molecule can comprise a 5′ cap, which is a naturally occurring 5′ cap or a 5′ cap analog. In an embodiment, the 5′ cap analog can be one of ARCA, beta-S-ARCA, beta-S-ARCA (D1), beta-S-ARCA (D2), CleanCap, Cap0, Cap1 or AU (Cap1).

In an embodiment, where at least one of the uridines in the first and/or second RNA molecule is a modified uridine and wherein the RNA molecules comprises a 5′ cap, the 5′ cap has the sequence NpppNU, wherein the U in the 5′ cap is an unmodified uridine. Preferably the 5′ cap has the sequence NpppAU wherein the U in the 5′ cap is an unmodified uridine and the A can be a modified or unmodified adenosine nucleotide.

In an embodiment, the first and/or second RNA molecule comprises a 5′ cap comprising a Cap1 and a cap proximal sequence comprising positions +1, +2, +3, +4, and +5 of the RNA molecule(s), wherein:
(i) the Cap1 comprises m⁷G (5′)ppp(5′) (2′OMeN1) pN2, wherein N1 is position+1 of the RNA molecule, and N₂ is position+2 of the RNA molecule, and wherein N₁ and N₂ are each independently chosen from: A, C, G, or U; and
(ii) the cap proximal sequence comprises N₁ and N₂ of the Cap1, and:
   (a) a sequence selected from the group consisting of: A₃A₄X₅; C₃A₄X₅; A₃C₄A₅ and A₃U₄G₅; or
   (b) a sequence comprising: X₃Y₄X₅;
   wherein X₃ or X₅ is each independently chosen from A, G, C, or U; and wherein Y₄ is not C.

In an embodiment, the first and/or second RNA molecule can comprise a modified 5′ regulatory region of a self-replicating RNA virus, which modified regulatory region comprises a point mutation at one or more of positions 67, 244, 245, 246, 248 of the 5′ regulatory region (SEQ ID NO: 1). Preferably, the self-replicating RNA virus is an alphavirus. Also preferably, the 5′ regulatory region further comprises a point mutation at position 4 of the 5′ regulatory region (SEQ ID NO: 1). Most preferably, the point mutations are G4A, A67C, G244A, C245A, G246A, or C248A.

In an embodiment, the first and/or second RNA molecule can comprise a 5′ replication recognition sequence, which is characterized in that at least one initiation codon is removed compared to a native 5′ replication recognition sequence. In an embodiment, the 5′ replication recognition sequence comprises a sequence homologous to an open reading frame of a non-structural protein or a portion thereof from a self-replicating virus, wherein the sequence homologous to an open reading frame of a non-structural protein or a portion thereof from a self-replicating virus is characterized in that it comprises the removal of at least one initiation codon compared to the native viral sequence. In an embodiment, the sequence homologous to an open reading frame of a non-structural protein or a portion thereof from a self-replicating virus is characterized in that it comprises the removal of at least the native start codon of the open reading frame of a non-structural protein from a self-replicating virus. In an embodiment, the sequence homologous to an open reading frame of a non-structural protein or a portion thereof from a self-replicating virus is characterized in that it comprises the removal of at least one initiation codon other than the native start codon of the open reading frame of a non-structural protein from a self-replicating virus. In an embodiment, the sequence homologous to an open reading frame of a non-structural protein or a portion thereof from a self-replicating virus is characterized in that it is free of initiation codons. The sequence homologous to an open reading frame of a non-structural protein or a portion thereof further comprises at least one nucleotide change compensating for nucleotide pairing disruptions within at least one stem loop introduced by the removal of at least one initiation codon.

In an embodiment, wherein the first and/or second RNA molecule can comprise a 3′ replication recognition sequence. In an embodiment, the 5′ and/or 3′ replication recognition sequences can be derived from a self-replicating virus, preferably the same self-replicating virus species. In an embodiment, the 5′ and/or 3′ replication recognition sequences can be derived from a self-replicating single-stranded RNA virus, such as a positive-sense, single-stranded RNA virus (e.g., alphavirus, flavivirus, etc.), preferably the same self-replicating virus species.

In an embodiment, the first and/or second RNA molecule can comprise an interrupted poly(A) sequence.

An exemplary poly(A) sequence is depicted in SEQ ID NO: 42. In an embodiment, a poly(A) sequence useful in the RNA molecules described herein is one that is at least 75%, 80%, 85%, 90%, 95%, 98% or 99% homologous to SEQ ID NO: 42.

In an embodiment, the first and/or second RNA molecule does not comprise an open reading frame for an intact virus structural protein.

In an embodiment, the system can further comprise a third or more replicable RNA molecules that can be replicated by the replicase encoded by the first RNA molecule. All embodiments described herein for the second RNA molecule also can apply to a third or further replicable RNA molecule.

In an embodiment, the system further can comprise a reagent capable of forming particles with the RNA molecules. In an embodiment, the reagent can be or comprise a polyalkyleneimine or a lipid. In an embodiment, the reagent can be or comprise a lipid, preferably comprising a cationic headgroup. In an embodiment, the reagent can be or comprise a pH responsive lipid. In an embodiment, the reagent can be or comprise a PEGylated-lipid. In an embodiment, the reagent can be conjugated to polysarcosine (pSar), poly(oxazoline) (POX); poly(oxazine) (POZ), poly(vinyl pyrrolidone) (PVP); poly(N-(2-hydroxypropyl)-methacrylamide) (pHPMA); poly(dehydroalanine) (pDha); poly(aminoethoxy ethoxy acetic acid) (pAEEA) or poly(2-methylaminoethoxy ethoxy acetic acid) (pmAEEA). Thus, the reagent can be or comprise a “grafted” or “stealth” lipid, i.e., a lipid conjugated to a polymer selected from the group consisting of: polyethylene-glycol (PEG); poly(aminoethoxy ethoxy acetic acid) (pAEEA), polysarcosine (pSar), poly(2-methylaminoethoxy ethoxy acetic acid) (pmAEEA); poly(oxazoline) (POX); poly(oxazine) (POZ), poly(vinyl pyrrolidone) (PVP); poly(N-(2-hydroxypropyl)-methacrylamide) (pHPMA); and poly(dehydroalanine) (pDha). The reagent can be or comprise a lipid conjugated to pAEEA or pSar. In some cases, the reagent does not comprise a lipid conjugated to PEG.

In an embodiment, the particles formed from the RNA molecules and the reagent can be lipid nanoparticles (LNP), lipoplexes (LPX), liposomes, or polymer-based polyplexes (PLX).

In an embodiment, the particle can further comprise at least one phosphatidylserine.

In an embodiment, the particles can be nanoparticles, in which:
(i) the number of positive charges in the nanoparticles does not exceed the number of negative charges in the nanoparticles and/or
(ii) the nanoparticles have a neutral or net negative charge and/or
(iii) the charge ratio of positive charges to negative charges in the nanoparticles is 1.4:1 or less and/or
(iv) the zeta potential of the nanoparticles is 0 or less.

Preferably, the charge ratio of positive charges to negative charges in the nanoparticles is between 1.4:1 and 1:8, preferably between 1.2:1 and 1:4.

In an embodiment, the nanoparticles can comprise at least one lipid, preferably comprise at least one cationic lipid. In an embodiment, the positive charges are contributed by the at least one cationic lipid and the negative charges are contributed by the RNA molecules. In an embodiment, the nanoparticles further can comprise at least one helper lipid. Preferably the helper lipid is a neutral lipid.

In an embodiment, the at least one cationic lipid comprises 1,2-di-O-octadecenyl-3-trimethylammonium propane (DOTMA), 1,2-dioleyloxy-3-dimethylaminopropane (DODMA), and/or 1,2-dioleoyl-3-trimethylammonium-propane (DOTAP). In an embodiment, the at least one helper lipid comprises 1,2-di-(9Z-octadecenoyl)-sn-glycero-3-phosphoethanolamine (DOPE), cholesterol (Chol), 1,2-dioleoyl-sn-glycero-3-phosphocholine (DOPC), and/or 1,2-distearoyl-sn-glycero-3-phosphocholine (DSPC). In an embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1.

In an embodiment, the nanoparticles are lipoplexes comprising DODMA and DOPE in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles are lipoplexes comprising DODMA and Cholesterol in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles are lipoplexes comprising DODMA and DSPC in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about lipoplexes 1.2:2. In an embodiment, the nanoparticles are comprising DODMA:Cholesterol:DOPE:PEGcerC16 in a molar ratio of 40:48:10:2.

In an embodiment, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In an embodiment, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In an embodiment, the reagent can comprise a lipid and the particles formed are LNPs which are complexed with and/or encapsulate the nucleic acid molecules, e.g., RNA molecules. In an embodiment, the reagent can comprise a lipid and the particles formed are vesicles encapsulating the nucleic acid molecules, e.g., RNA molecules, optionally unilamellar liposomes. In an embodiment the composition comprising the nucleic acid molecule is an LNP composition, such as an RNA-LNP composition. The reagent capable of forming particles with the nucleic acid molecules can be or comprise a cationically ionizable lipid, neutral (e.g., helper) lipid, a steroid (e.g., cholesterol), and a polymer conjugated lipid.

In an embodiment, the reagent can be or comprise polyalkyleneimine.

In an embodiment, the molar ratio of the number of nitrogen atoms (N) in the polyalkyleneimine to the number of phosphor atoms (P) in the RNA molecules (N:P ratio) can be 2.0 to 15.0, preferably 6.0 to 12.0. In an embodiment, the molar ratio of the number of the number of nitrogen atoms (N) in the polyalkyleneimine to the number of phosphor atoms (P) in the RNA molecules (N:P ratio) can be at least about 48, optionally about 48 to 300, about 60 to 200, or about 80 to 150.

In an embodiment, the ionic strength of the composition can be 50 mM or less, preferably wherein the concentration of monovalent cationic ions can be 25 mM or less and the concentration of divalent cationic ions can be 20 UM or less.

In an embodiment, the particles formed are polyplexes.

In an embodiment, the polyalkyleneimine comprises the following general formula (I):

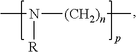

wherein
R is H, an acyl group or a group comprising the following general formula (II):

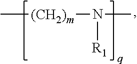

wherein R¹ is H or a group comprising the following general formula (III):

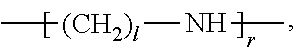

n, m, and l are independently selected from integers from 2 to 10; and
p, q, and r are integers, wherein the sum of p, q, and r is such that the average molecular weight of the polymer is 1.5.10² to 10⁷ Da, preferably 5000 to 10⁵ Da, more preferably 10000 to 40000 Da, more preferably 15000 to 30000 Da, even more preferably 20000 to 25000 Da. Preferably n, m, and l are independently selected from 2, 3, 4, and 5, preferably from 2 and 3. Preferably R¹ is H. Preferably R is H or an acyl group.

In an embodiment, the polyalkyleneimine can comprise polyethylenimine and/or polypropylenimine, preferably polyethyleneimine. In an embodiment, at least 92% of the N atoms in the polyalkyleneimine are protonatable.

In an embodiment, the system further can comprise one or more peptide-based adjuvants, wherein peptide-based adjuvants optionally comprise immune regulatory molecules, such as cytokines, lymphokines and/or co-stimulatory molecules.

In an embodiment, the system further can comprise one or more additives, wherein the additives optionally are selected from the group consisting of buffering substances, saccharides, stabilizers, cryoprotectants, lyoprotectants, and chelating agents. Preferably the buffering substances comprise at least one selected from the group consisting of 4-(2-hydroxyethyl)-1-piperazineethanesulfonic acid (HEPES), 2-(N-morpholino) ethanesulfonic acid (MES), 3-morpholino-2-hydroxypropanesulfonic acid (MOPSO), acetic acid, acetate buffers and analogues, phosphoric acid and phosphate buffers, and citric acid and citrate buffers. Preferably the saccharides comprise at least one selected from the group consisting of monosaccharides, disaccharides, trisaccharides, oligosaccharides, and polysaccharides preferably from glucose, trehalose, and saccharose. Preferably the cryoprotectants comprise at least one selected from the group consisting of glycols, such as ethylene glycol, propylene glycol, and glycerol. Preferably the chelating agent comprises EDTA.

Also described herein is a kit comprising two RNA molecules, wherein the first RNA molecule comprises an open reading frame encoding a functional RNA-dependent RNA polymerase (replicase) and wherein the second RNA molecule is a replicable RNA molecule comprising at least one miRNA sequence, which miRNA sequence is capable of being excised from the second replicable RNA when present in a cell, and is capable of regulating gene expression in a cell, which replicable RNA molecule cis capable of being replicated in trans by the replicase encoded by the first RNA molecule. In an embodiment, the two RNA molecules are in separate containers contained within the kit.

Also described herein is a pharmaceutical composition comprising two RNA molecules, wherein the first RNA molecule comprises an open reading frame encoding a functional RNA-dependent RNA polymerase (replicase) and wherein the second RNA molecule is a replicable RNA molecule comprising at least one miRNA sequence, which miRNA sequence is capable of being excised from the second replicable RNA when present in a cell, and is capable of regulating gene expression in a cell, which replicable RNA molecule is capable of being replicated in trans by the replicase encoded by the first RNA molecule, and a pharmaceutically acceptable carrier.

In an embodiment, the first and/or second RNA molecule in the composition, preferably the second RNA molecule, further comprises at least one open reading frame (ORF) encoding a protein of interest.

In an embodiment, the pharmaceutical composition can be formulated for intradermal, subcutaneous, and/or intramuscular administration, such as by injection. In an embodiment, the kit or the pharmaceutical composition can be for use in therapy. In an embodiment, the kit or the pharmaceutical composition can be for use in a method of treating or preventing a disease, preferably wherein the subject is a mammal, more preferably wherein the mammal is a human, said method comprising administering a pharmaceutical composition according to the invention to the subject. Preferably, administering the kit or the pharmaceutical composition comprises intradermal, subcutaneous, or intramuscular administration, such as by intradermal, subcutaneous or intramuscular injection. The injection is by use of a needle or is by use of a needleless injection device. Preferably, administering comprises administration by intramuscular injection, preferably with a needle. In an embodiment, the RNA molecules are administered separately, preferably by the same rout of administration.

In an embodiment, the disease is a bacterial, viral, parasitical or fungal infection, or cancer. The subject is preferably a human.

Also described herein is a method for the treatment or prevention of a bacterial, viral, parasitical or fungal infection in a subject, said method comprising administering to the subject a composition described herein, preferably a pharmaceutical composition. Also described herein is a method for the treatment or prevention of cancer in a subject, said method comprising administering to the subject a composition described herein, preferably a pharmaceutical composition.

Also described herein is a first RNA molecule and a second RNA molecule for use in therapy, wherein the first RNA molecule comprises an open reading frame encoding a functional RNA-dependent RNA polymerase (replicase) and the second RNA molecule is a replicable RNA molecule comprising at least one miRNA sequence, which miRNA sequence is capable of being excised from the second replicable RNA when present in a cell, and is capable of regulating gene expression in a cell, and which replicable RNA molecule is capable of being replicated in trans by the replicase encoded by the first RNA molecule. Preferably, the therapy is the treatment or prevention of cancer or an infectious disease.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

The term “about” means approximately or nearly, and in the context of a numerical value or range set forth herein preferably means+/−10% of the numerical value or range recited or claimed.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

Terms such as “reduce” or “inhibit” as used herein means the ability to cause an overall decrease, preferably of 5% or greater, 10% or greater, 20% or greater, more preferably of 50% or greater, and most preferably 75% or greater, in the level. The term “inhibit” or similar phrases includes a complete or essentially complete inhibition, i.e., a reduction to zero or essentially to zero.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%.

The term “net charge” refers to the charge on a whole object, such as a compound or particle.

An ion having an overall net positive charge is a cation, while an ion having an overall net negative charge is an anion. Thus, according to the invention, an anion is an ion with more electrons than protons, giving it a net negative charge; and a cation is an ion with fewer electrons than protons, giving it a net positive charge.

Terms as “charged”, “net charge”, “negatively charged” or “positively charged”, with reference to a given compound or particle, refer to the electric net charge of the given compound or particle when dissolved or suspended in water at pH 7.0.

The term “nucleic acid” according to the invention also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the invention, nucleic acids comprise genomic DNA, cDNA, mRNA, viral RNA, recombinantly prepared and chemically synthesized molecules. According to the invention, a nucleic acid may be in the form of a single-stranded or double-stranded and linear or covalently closed circular molecule.

According to the invention “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g. a fragment) thereof.

According to the present invention, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the invention, RNA may be single-stranded or double-stranded. In some embodiments of the present invention, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

The term “translation efficiency” relates to the amount of translation product provided by an RNA molecule within a particular period of time.

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability and/or translational efficiency.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence.

The term “variant” with respect to, for example, nucleic acid and amino acid sequences, according to the invention includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

Nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

As described below, some embodiments of the present invention are characterized, inter alia, by nucleic acid sequences that are homologous to other nucleic acid sequences. These homologous sequences are variants of other nucleic acid sequences.

Preferably the degree of identity between a given nucleic acid sequence and a nucleic acid sequence which is a variant of said given nucleic acid sequence or between a given amino acid sequence of a protein and an amino acid sequence which is a variant of said given amino acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence similarity” indicates the percentage of amino acids that either are identical or that represent conservative amino acid substitutions. “Sequence identity” between two amino acid or nucleic acid sequences indicates the percentage of amino acids or nucleotides that are identical between the sequences.

The term “% identical” is intended to refer, in particular, to a percentage of amino acids or nucleotides which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, J. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another. According to the invention, hybridization is preferably carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York and refer, for example, to hybridization at 65 °C in hybridization buffer (3.5×SSC, 0.02% Ficoll, 0.02% polyvinylpyrrolidone, 0.02% bovine serum albumin, 2.5 mM NaH₂PO₄ (PH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68 °C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the invention is at least 70%, preferably at least 75%, preferably at least 80%, more preferably at least 85%, even more preferably at least 90% or most preferably at least 95%, 96%, 97%, 98% or 99%. Most preferably, the degree of complementarity according to the invention is 100%.

The term “derivative” comprises any chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

A “nucleic acid sequence which is derived from a nucleic acid sequence” refers to a nucleic acid which is a variant of the nucleic acid from which it is derived. Preferably, a sequence which is a variant with respect to a specific sequence, when it replaces the specific sequence in an RNA molecule retains RNA stability and/or translational efficiency.

“nt” is an abbreviation for nucleotide; or for nucleotides, preferably consecutive nucleotides in a nucleic acid molecule.

According to the invention, the term “codon” refers to a base triplet in a coding nucleic acid that specifies which amino acid will be added next during protein synthesis at the ribosome.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an alphaviral nucleic acid template, the template is typically RNA. Subsequently, the transcribed RNA may be translated into protein. According to the present invention, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single-stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

“3′ end of a nucleic acid” refers according to the invention to that end which has a free hydroxy group. In a diagrammatic representation of double-stranded nucleic acids, in particular DNA, the 3′ end is always on the right-hand side. “5′ end of a nucleic acid” refers according to the invention to that end which has a free phosphate group. In a diagrammatic representation of double-strand nucleic acids, in particular DNA, the 5′ end is always on the left-hand side.

| | | |
|---|---|---|
| 5′ end | 5′--P-NNNNNNN-OH-3′ | 3′ end |
| | 3′-HO-NNNNNNN-P--5′ | |

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5” of that second element. For a double-stranded nucleic acid molecule, indications like “upstream” and “downstream” are given with respect to the (+) strand.

According to the invention, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript).

In particular embodiments, a nucleic acid is functionally linked according to the invention to expression control sequences which may be homologous or heterologous with respect to the nucleic acid.

The term “expression control sequence” comprises according to the invention promoters, ribosome-binding sequences and other control elements which control transcription of a gene or translation of the derived RNA. In particular embodiments of the invention, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like. An expression control sequence of alphaviral RNA may include a subgenomic promoter and/or one or more conserved sequence element(s). A specific expression control sequence according to the present invention is a subgenomic promoter of an alphavirus, as described herein.

The nucleic acid sequences specified herein, in particular transcribable and coding nucleic acid sequences, may be combined with any expression control sequences, in particular promoters, which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A transcribable nucleic acid sequence, in particular a nucleic acid sequence coding for a peptide or protein, and an expression control sequence are “functionally” linked to one another, if they are covalently linked to one another in such a way that transcription or expression of the transcribable and in particular coding nucleic acid sequence is under the control or under the influence of the expression control sequence. If the nucleic acid sequence is to be translated into a functional peptide or protein, induction of an expression control sequence functionally linked to the coding sequence results in transcription of said coding sequence, without causing a frame shift in the coding sequence or the coding sequence being unable to be translated into the desired peptide or protein.

The term “promoter” or “promoter region” refers to a nucleic acid sequence which controls synthesis of a transcript, e.g. a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. A specific promoter according to the present invention is a subgenomic promoter, e.g., of an alphavirus, as described herein. An exemplary subgenomic promoter is depicted in SEQ ID NO: 46. In an embodiment, a subgenomic promoter useful in the RNA molecules described herein is one that is at least 85%, 90%, 95%, 98% or 99% homologous to SEQ ID NO: 46. Other specific promoters are genomic plus-strand or negative-strand promoters, e.g., of an alphavirus.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

An “RNA-dependent RNA polymerase” or “RdRP”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. RNA-dependent RNA polymerase is thus synonymously referred to as “RNA replicase” or simply “replicase”. In nature, RNA-dependent RNA polymerases are typically encoded by all RNA viruses except retroviruses. Typical representatives of viruses encoding an RNA-dependent RNA polymerase are alphaviruses.

According to the present invention, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by a RNA-dependent RNA polymerase (RdRP): a template RNA strand (first RNA strand)—or a part thereof-serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the invention, the term “template RNA” refers to RNA that can be transcribed or replicated by an RNA-dependent RNA polymerase.

According to the invention, the term “gene” refers to a particular nucleic acid sequence which is responsible for producing one or more cellular products and/or for achieving one or more intercellular or intracellular functions.

More specifically, said term relates to a nucleic acid section (typically DNA; but RNA in the case of RNA viruses) which comprises a nucleic acid coding for a specific protein or a functional or structural RNA molecule.

An “isolated molecule” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the invention, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g. messenger RNA) directs the assembly of a sequence of amino acids to make a peptide or protein.

According to the invention, the term “mRNA” means “messenger-RNA” and relates to a transcript which encodes a peptide or protein. The mRNA is translated such that the encoded peptide or protein is produced. mRNA may also refer more broadly to a transcript which does not get translated but which encodes/provides a functional nucleotide sequence, such as a a miRNA or other non-coding RNA species. Typically, mRNA comprises a 5′-UTR, a protein coding region, a 3′-UTR, and a poly(A) sequence. Replicable RNA molecules, such as self-amplifying RNA (saRNA) or cis-replicons, or trans-replicons (TRs) or nano-transreplicons (NTRs) may be understood to be a type of mRNA regardless of whether they are actually translated. mRNA may be generated by in vitro transcription from a DNA template. The in vitro transcription methodology is known to the skilled person. For example, there is a variety of in vitro transcription kits commercially available. According to the invention, mRNA may be modified by stabilizing modifications and capping.

According to the invention, the terms “poly(A) sequence” or “poly(A) tail” or “poly(A) structure” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. While a poly(A) sequence is normally not encoded in eukaryotic DNA, but is attached during eukaryotic transcription in the cell nucleus to the free 3′ end of the RNA by a template-independent RNA polymerase after transcription, the present invention encompasses poly(A) sequences encoded by DNA. In a preferred embodiment, the RNA molecules described herein comprise an uninterrupted poly(A)-sequence.

According to the invention, the term “primary structure”, with reference to a nucleic acid molecule, refers to the linear sequence of nucleotide monomers.

According to the invention, the term “secondary structure”, with reference to a nucleic acid molecule, refers to a two-dimensional representation of a nucleic acid molecule that reflects base pairings; e.g.; in the case of a single-stranded RNA molecule particularly intramolecular base pairings. Although each RNA molecule has only a single polynucleotide chain, the molecule is typically characterized by regions of (intramolecular) base pairs. According to the invention, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., 2006; J. Graph Algorithms Appl. 10:329-351). As described herein, the secondary structure of certain RNA molecules is relevant in the context of the present invention.

According to the invention, secondary structure of a nucleic acid molecule, particularly of a single-stranded RNA molecule, is determined by prediction using the web server for RNA secondary structure prediction (http://rna.urmc.rochester.edu/RNAstructureWeb/Servers/Predict1/Predict1.html). Preferably, according to the invention, “secondary structure”, with reference to a nucleic acid molecule, specifically refers to the secondary structure determined by said prediction. The prediction may also be performed or confirmed using MFOLD structure prediction (http://unafold.rna.albany.edu/?q=mfold).

According to the invention, a “base pair” is a structural motif of a secondary structure wherein two nucleotide bases associate with each other through hydrogen bonds between donor and acceptor sites on the bases. The complementary bases, A:U and G:C, form stable base pairs through hydrogen bonds between donor and acceptor sites on the bases; the A:U and G:C base pairs are called Watson-Crick base pairs. A weaker base pair (called Wobble base pair) is formed by the bases G and U (G:U). The base pairs A:U and G:C are called canonical base pairs. Other base pairs like G:U (which occurs fairly often in RNA) and other rare base-pairs (e.g. A:C; U:U) are called non-canonical base pairs.

According to the invention, “nucleotide pairing” refers to two nucleotides that associate with each other so that their bases form a base pair (canonical or non-canonical base pair, preferably canonical base pair, most preferably Watson-Crick base pair).

According to the invention, the terms “stem loop” or “hairpin” or “hairpin loop”, with reference to a nucleic acid molecule, all interchangeably refer to a particular secondary structure of a nucleic acid molecule, typically a single-stranded nucleic acid molecule, such as single-stranded RNA. The particular secondary structure represented by the stem loop consists of a consecutive nucleic acid sequence comprising a stem and a (terminal) loop, also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as, e.g., stem loop elements stem 1 and stem 2. The stem loop is formed when these two neighbored entirely or partially reverse complementary sequences, e.g. stem loop elements stem 1 and stem 2, form base-pairs with each other, leading to a double stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule—form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure requires the presence of a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present invention, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by a stem loop, the respective complementary nucleic acid sequence is typically also characterized by a stem loop. A stem loop is typically formed by single-stranded RNA molecules. For example, several stem loops are present in the 5′ replication recognition sequence of alphaviral genomic RNA.

According to the invention, “disruption” or “disrupt”, with reference to a specific secondary structure of a nucleic acid molecule (e.g., a stem loop) means that the specific secondary structure is absent or altered. Typically, a secondary structure may be disrupted as a consequence of a change of at least one nucleotide that is part of the secondary structure. For example, a stem loop may be disrupted by change of one or more nucleotides that form the stem, so that nucleotide pairing is not possible.

According to the invention, “compensates for secondary structure disruption” or “compensating for secondary structure disruption” refers to one or more nucleotide changes in a nucleic acid sequence; more typically it refers to one or more second nucleotide changes in a nucleic acid sequence, which nucleic acid sequence also comprises one or more first nucleotide changes, characterized as follows: while the one or more first nucleotide changes, in the absence of the one or more second nucleotide changes, cause a disruption of the secondary structure of the nucleic acid sequence, the co-occurrence of the one or more first nucleotide changes and the one or more second nucleotide changes does not cause the secondary structure of the nucleic acid to be disrupted. Co-occurrence means presence of both the one or more first nucleotide changes and of the one or more second nucleotide changes. Typically, the one or more first nucleotide changes and the one or more second nucleotide changes are present together in the same nucleic acid molecule. In a specific embodiment, one or more nucleotide changes that compensate for secondary structure disruption is/are one or more nucleotide changes that compensate for one or more nucleotide pairing disruptions. Thus, in one embodiment, “compensating for secondary structure disruption” means “compensating for nucleotide pairing disruptions”, i.e. one or more nucleotide pairing disruptions, for example one or more nucleotide pairing disruptions within one or more stem loops. The one or more one or more nucleotide pairing disruptions may have been introduced by the removal of at least one initiation codon. Each of the one or more nucleotide changes that compensates for secondary structure disruption is a nucleotide change, which can each be independently selected from a deletion, an addition, a substitution and/or an insertion of one or more nucleotides. In an illustrative example, when the nucleotide pairing A:U has been disrupted by substitution of A to C (C and U are not typically suitable to form a nucleotide pair); then a nucleotide change that compensates for nucleotide pairing disruption may be substitution of U by G, thereby enabling formation of the C:G nucleotide pairing. The substitution of U by G thus compensates for the nucleotide pairing disruption. In an alternative example, when the nucleotide pairing A:U has been disrupted by substitution of A to C; then a nucleotide change that compensates for nucleotide pairing disruption may be substitution of C by A, thereby restoring formation of the original A:U nucleotide pairing. In general, in the present invention, those nucleotide changes compensating for secondary structure disruption are preferred which do neither restore the original nucleic acid sequence nor create novel AUG triplets. In the above set of examples, the U to G substitution is preferred over the C to A substitution.

According to the invention, the term “tertiary structure”, with reference to a nucleic acid molecule, refers to the three-dimensional structure of a nucleic acid molecule, as defined by the atomic coordinates.

According to the invention, a nucleic acid such as RNA, e.g., rRNA, may encode a protein. Accordingly, a transcribable nucleic acid sequence or a transcript thereof may contain an open reading frame (ORF) encoding a protein.

According to the invention, the term “nucleic acid encoding a protein” means that the nucleic acid, if present in the appropriate environment, preferably within a cell, can direct the assembly of amino acids to produce the protein during the process of translation. Preferably, coding RNA according to the invention is able to interact with the cellular translation machinery allowing translation of the coding RNA to yield a protein.

According to the invention, the term “peptide” comprises oligo- and polypeptides and refers to substances which comprise two or more, preferably 3 or more, preferably 4 or more, preferably 6 or more, preferably 8 or more, preferably 10 or more, preferably 13 or more, preferably 16 or more, preferably 20 or more, and up to preferably 50, preferably 100 or preferably 150, consecutive amino acids linked to one another via peptide bonds. The terms “peptide” and “protein” are used herein usually as synonyms.

The terms “peptide” and “protein” comprise, according to the invention, substances which contain not only amino acid components but also non-amino acid components such as sugars and phosphate structures, and also comprise substances containing bonds such as ester, thioether or disulfide bonds.

According to the invention, the term “polyprotein” refers to a single peptide, which comprises the amino acid sequences for at least 2, preferably at least 3, preferably at least 4, proteins, preferably as an intermediate. The single peptide is cleaved by proteases to produce the single proteins. The proteins included in the polyprotein can already function within the context of the polyprotein or can gain a function upon cleavage from the polyprotein. In addition, the function of a protein may change upon cleavage from the polyprotein. The proteases cleaving the polyprotein can be included in the polyprotein itself, i.e. the polyprotein has auto-proteolytic activity. The polyprotein is usually produced by translation of a single open reading frame of an RNA.

According to the invention, the terms “initiation codon” and “start codon” synonymously refer to a codon (base triplet) of an RNA molecule that is potentially the first codon that is translated by a ribosome. Such codon typically encodes the amino acid methionine in eukaryotes and a modified methionine in prokaryotes. The most common initiation codon in eukaryotes and prokaryotes is AUG. Unless specifically stated herein that an initiation codon other than AUG is meant, the terms “initiation codon” and “start codon”, with reference to an RNA molecule, refer to the codon AUG. According to the invention, the terms “initiation codon” and “start codon” are also used to refer to a corresponding base triplet of a deoxyribonucleic acid, namely the base triplet encoding the initiation codon of an RNA. If the initiation codon of messenger RNA is AUG, the base triplet encoding the AUG is ATG. According to the invention, the terms “initiation codon” and “start codon” preferably refer to a functional initiation codon or start codon, i.e., to an initiation codon or start codon that is used or would be used as a codon by a ribosome to start translation. There may be AUG codons in an RNA molecule that are not used as codons by a ribosome to start translation, e.g., due to a short distance of the codons to the cap. These codons are not encompassed by the term functional initiation codon or start codon.

According to the invention, the terms “start codon of the open reading frame” or “initiation codon of the open reading frame” refer to the base triplet that serves as initiation codon for protein synthesis in a coding sequence, e.g., in the coding sequence of a nucleic acid molecule found in nature. In an RNA molecule, the start codon of the open reading frame is often preceded by a 5′ untranslated region (5′-UTR), although this is not strictly required.

According to the invention, the terms “native start codon of the open reading frame” or “native initiation codon of the open reading frame” refer to the base triplet that serves as initiation codon for protein synthesis in a native coding sequence. A native coding sequence may be, e.g., the coding sequence of a nucleic acid molecule found in nature. In some embodiments, the present invention provides variants of nucleic acid molecules found in nature, which are characterized in that the native start codon (which is present in the native coding sequence) has been removed (so that it is not present in the variant nucleic acid molecule).

According to the invention, “first AUG” means the most upstream AUG base triplet of a messenger RNA molecule, preferably the most upstream AUG base triplet of a messenger RNA molecule that is used or would be used as a codon by a ribosome to start translation. Accordingly, “first ATG” refers to the ATG base triplet of a coding DNA sequence that encodes the first AUG. In some instances, the first AUG of a mRNA molecule is the start codon of an open reading frame, i.e., the codon that is used as start codon during ribosomal protein synthesis.

According to the invention, the terms “comprises the removal” or “characterized by the removal” and similar terms, with reference to a certain element of a nucleic acid variant, mean that said certain element is not functional or not present in the nucleic acid variant, compared to a reference nucleic acid molecule. Without limitation, a removal can consist of deletion of all or part of the certain element, of substitution of all or part of the certain element, or of alteration of the functional or structural properties of the certain element. The removal of a functional element of a nucleic acid sequence requires that the function is not exhibited at the position of the nucleic acid variant comprising the removal. For example, an RNA variant characterized by the removal of a certain initiation codon requires that ribosomal protein synthesis is not initiated at the position of the RNA variant characterized by the removal. The removal of a structural element of a nucleic acid sequence requires that the structural element is not present at the position of the nucleic acid variant comprising the removal. For example, a RNA variant characterized by the removal of a certain AUG base triplet, i.e., of a AUG base triplet at a certain position, may be characterized, e.g., by deletion of part or all of the certain AUG base triplet (e.g., AAUG), or by substitution of one or more nucleotides (A, U, G) of the certain AUG base triplet by any one or more different nucleotides, so that the resulting nucleotide sequence of the variant does not comprise said AUG base triplet. Suitable substitutions of one nucleotide are those that convert the AUG base triplet into a GUG, CUG or UUG base triplet, or into a AAG, ACG or AGG base triplet, or into a AUA, AUC or AUU base triplet. Suitable substitutions of more nucleotides can be selected accordingly.

According to the invention, the term “self-replicating virus” includes RNA viruses capable of replicating autonomously in a host cell. Self-replicating viruses may have a single-stranded RNA (ssRNA) genome and include alphaviruses, flaviviruses, measles viruses (MVs) and rhabdoviruses. Alphaviruses and flaviviruses possess a genome of positive polarity, whereas the genome of measles viruses (MVs) and rhabdoviruses is negative strand SSRNA. Typically, a self-replicating virus is a virus with a (+) stranded RNA genome which can be directly translated after infection of a cell, and this translation provides an RNA-dependent RNA polymerase which then produces both antisense and sense transcripts from the infected RNA. In the following, the invention is illustrated by referring to alphavirus-derived vectors as an example of self-replicating virus-derived vectors. However, it is to be understood that the present invention is not limited to alphavirus-derived vectors.

According to the invention, the term “alphavirus” is to be understood broadly and includes any virus particle that has characteristics of alphaviruses. Characteristics of alphavirus include the presence of a (+) stranded RNA which encodes genetic information suitable for replication in a host cell, including RNA polymerase activity. Further characteristics of many alphaviruses are described, e.g., in Strauss & Strauss, 1994, Microbiol. Rev. 58:491-562. The term “alphavirus” includes alphavirus found in nature, as well as any variant or derivative thereof. In some embodiments, a variant or derivative is not found in nature.

In one embodiment, the alphavirus is an alphavirus found in nature. Typically, an alphavirus found in nature is infectious to any one or more eukaryotic organisms, such as an animal (including a vertebrate such as a human, and an arthropod such as an insect). An alphavirus found in nature is preferably selected from the group consisting of the following: Barmah Forest virus complex (comprising Barmah Forest virus); Eastern equine encephalitis complex (comprising seven antigenic types of Eastern equine encephalitis virus); Middelburg virus complex (comprising Middelburg virus); Ndumu virus complex (comprising Ndumu virus); Semliki Forest virus complex (comprising Bebaru virus, Chikungunya virus, Mayaro virus and its subtype Una virus, O'Nyong Nyong virus, and its subtype Igbo-Ora virus, Ross River virus and its subtypes Bebaru virus, Getah virus, Sagiyama virus, Semliki Forest virus and its subtype Me Tri virus); Venezuelan equine encephalitis complex (comprising Cabassou virus, Everglades virus, Mosso das Pedras virus, Mucambo virus, Paramana virus, Pixuna virus, Rio Negro virus, Trocara virus and its subtype Bijou Bridge virus, Venezuelan equine encephalitis virus); Western equine encephalitis complex (comprising Aura virus, Babanki virus, Kyzylagach virus, Sindbis virus, Ockelbo virus, Whataroa virus, Buggy Creek virus, Fort Morgan virus, Highlands J virus, Western equine encephalitis virus); and some unclassified viruses including Salmon pancreatic disease virus; Sleeping Disease virus; Southern elephant seal virus; Tonate virus. More preferably, the alphavirus is selected from the group consisting of Semliki Forest virus complex (comprising the virus types as indicated above, including Semliki Forest virus), Western equine encephalitis complex (comprising the virus types as indicated above, including Sindbis virus), Eastern equine encephalitis virus (comprising the virus types as indicated above), Venezuelan equine encephalitis complex (comprising the virus types as indicated above, including Venezuelan equine encephalitis virus).

In a further preferred embodiment, the alphavirus is Semliki Forest virus. In an alternative further preferred embodiment, the alphavirus is Sindbis virus. In an alternative further preferred embodiment, the alphavirus is Venezuelan equine encephalitis virus.

In some embodiments of the present invention, the alphavirus is not an alphavirus found in nature. Typically, an alphavirus not found in nature is a variant or derivative of an alphavirus found in nature, that is distinguished from an alphavirus found in nature by at least one mutation in the nucleotide sequence, i.e., the genomic RNA. The mutation in the nucleotide sequence may be selected from an insertion, a substitution or a deletion of one or more nucleotides, compared to an alphavirus found in nature. A mutation in the nucleotide sequence may or may not be associated with a mutation in a polypeptide or protein encoded by the nucleotide sequence. For example, an alphavirus not found in nature may be an attenuated alphavirus. An attenuated alphavirus not found in nature is an alphavirus that typically has at least one mutation in its nucleotide sequence by which it is distinguished from an alphavirus found in nature, and that is either not infectious at all, or that is infectious but has a lower disease-producing ability or no disease-producing ability at all. As an illustrative example, TC83 is an attenuated alphavirus that is distinguished from the Venezuelan equine encephalitis virus (VEEV) found in nature (Mckinney et al., 1963, Am. J. Trop. Med. Hyg. 12:597-603).

Members of the alphavirus genus may also be classified based on their relative clinical features in humans: alphaviruses associated primarily with encephalitis, and alphaviruses associated primarily with fever, rash, and polyarthritis.

The term “alphaviral” means found in an alphavirus, or originating from an alphavirus or derived from an alphavirus, e.g., by genetic engineering.

According to the invention, “SFV” stands for Semliki Forest virus. According to the invention, “SIN” or “SINV” stands for Sindbis virus. According to the invention, “VEE” or “VEEV” stands for Venezuelan equine encephalitis virus.

According to the invention, the term “of an alphavirus” refers to an entity of origin from an alphavirus. For illustration, a protein of an alphavirus may refer to a protein that is found in alphavirus and/or to a protein that is encoded by alphavirus; and a nucleic acid sequence of an alphavirus may refer to a nucleic acid sequence that is found in alphavirus and/or to a nucleic acid sequence that is encoded by alphavirus. Preferably, a nucleic acid sequence “of an alphavirus” refers to a nucleic acid sequence “of the genome of an alphavirus” and/or “of genomic RNA of an alphavirus”.

According to the invention, the term “alphaviral RNA” refers to any one or more of alphaviral genomic RNA (i.e., (+) strand), complement of alphaviral genomic RNA (i.e., (−) strand), and the subgenomic transcript (i.e. (+) strand), or a fragment of any thereof.

According to the invention, “alphavirus genome” refers to genomic (+) strand RNA of an alphavirus.

According to the invention, the term “native alphavirus sequence” and similar terms typically refer to a (e.g., nucleic acid) sequence of a naturally occurring alphavirus (alphavirus found in nature). In some embodiments, the term “native alphavirus sequence” also includes a sequence of an attenuated alphavirus.

According to the invention, the term “5′ replication recognition sequence” preferably refers to a continuous nucleic acid sequence, preferably a ribonucleic acid sequence, that is identical or homologous to a 5′ fragment of a genome of a self-replicating virus, such as an alphavirus genome. The “5′ replication recognition sequence” is a nucleic acid sequence that can be recognized by a replicase such as an alphaviral replicase. The term 5′ replication recognition sequence includes native 5′ replication recognition sequences as well as functional equivalents thereof, such as, e.g., functional variants of a 5′ replication recognition sequence of a self-replicating virus found in nature, e.g., alphavirus found in nature. According to the invention, functional equivalents include derivatives of 5′ replication recognition sequences characterized by the removal of at least one initiation codon as described herein. The 5′ replication recognition sequence is required for synthesis of the (−) strand complement of alphavirus genomic RNA, and is required for synthesis of (+) strand viral genomic RNA based on a (−) strand template. A native 5′ replication recognition sequence typically encodes at least the N-terminal fragment of nsP1; but does not comprise the entire open reading frame encoding nsP1234. In view of the fact that a native 5′ replication recognition sequence typically encodes at least the N-terminal fragment of nsP1, a native 5′ replication recognition sequence typically comprises at least one initiation codon, typically AUG. In one embodiment, the 5′ replication recognition sequence comprises conserved sequence element 1 of an alphavirus genome (CSE 1) or a variant thereof and conserved sequence element 2 of an alphavirus genome (CSE 2) or a variant thereof. The 5′ replication recognition sequence is typically capable of forming four stem loops (SL), i.e. SL1, SL2, SL3, SL4. The numbering of these stem loops begins at the 5′ end of the 5′ replication recognition sequence.

The term “conserved sequence element” or “CSE” refers to a nucleotide sequence found in alphavirus RNA. These sequence elements are termed “conserved” because orthologs are present in the genome of different alphaviruses, and orthologous CSEs of different alphaviruses preferably share a high percentage of sequence identity and/or a similar secondary or tertiary structure. The term CSE includes CSE 1, CSE 2, CSE 3 and CSE 4.

According to the invention, the terms “CSE 1” or “44-nt CSE” synonymously refer to a nucleotide sequence that is required for (+) strand synthesis from a (−) strand template. The term “CSE 1” refers to a sequence on the (+) strand; and the complementary sequence of CSE 1 (on the (−) strand) functions as a promoter for (+) strand synthesis. Preferably, the term CSE 1 includes the most 5′ nucleotide of the alphavirus genome. CSE 1 typically forms a conserved stem-loop structure. Without wishing to be bound to a particular theory, it is believed that, for CSE 1, the secondary structure is more important than the primary structure, i.e., the linear sequence. In genomic RNA of the model alphavirus Sindbis virus, CSE 1 consists of a consecutive sequence of 44 nucleotides, which is formed by the most 5′ 44 nucleotides of the genomic RNA (Strauss & Strauss, 1994, Microbiol. Rev. 58:491-562).

According to the invention, the terms “CSE 2” or “51-nt CSE” synonymously refer to a nucleotide sequence that is required for (−) strand synthesis from a (+) strand template. The (+) strand template is typically alphavirus genomic RNA or an RNA replicon (note that the subgenomic RNA transcript, which does not comprise CSE 2, does not function as a template for (−) strand synthesis). In alphavirus genomic RNA, CSE 2 is typically localized within the coding sequence for nsP1. In genomic RNA of the model alphavirus Sindbis virus, the 51-nt CSE is located at nucleotide positions 155-205 of genomic RNA (Frolov et al., 2001, RNA, vol. 7, pp. 1638-1651). CSE 2 forms typically two conserved stem loop structures. These stem loop structures are designated as stem loop 3 (SL3) and stem loop 4 (SL4) because they are the third and fourth conserved stem loop, respectively, of alphavirus genomic RNA, counted from the 5′ end of alphavirus genomic RNA. Without wishing to be bound to a particular theory, it is believed that, for CSE 2, the secondary structure is more important than the primary structure, i.e. the linear sequence.

According to the invention, the terms “CSE 3” or “junction sequence” synonymously refer to a nucleotide sequence that is derived from alphaviral genomic RNA and that comprises the start site of the subgenomic RNA. The complement of this sequence in the (−) strand acts to promote subgenomic RNA transcription. In alphavirus genomic RNA, CSE 3 typically overlaps with the region encoding the C-terminal fragment of nsP4 and extends to a short non-coding region located upstream of the open reading frame encoding the structural proteins.

According to the invention, the terms “CSE 4” or “19-nt conserved sequence” or “19-nt CSE” synonymously refer to a nucleotide sequence from alphaviral genomic RNA, immediately upstream of the poly(A) sequence in the 3′ untranslated region of the alphavirus genome. CSE 4 typically consists of 19 consecutive nucleotides. Without wishing to be bound to a particular theory, CSE 4 is understood to function as a core promoter for initiation of (−) strand synthesis (José et al., 2009, Future Microbiol. 4:837-856); and/or CSE 4 and the poly(A) sitail of the alphavirus genomic RNA are understood to function together for efficient (−) strand synthesis (Hardy & Rice, 2005, J. Virol. 79:4630-4639).

According to the invention, the term “subgenomic promoter” or “SGP” refers to a nucleic acid sequence upstream (5′) of a nucleic acid sequence (e.g., coding sequence), which controls transcription of said nucleic acid sequence by providing a recognition and binding site for RNA polymerase, typically RNA-dependent RNA polymerase, in particular functional alphavirus non-structural protein. The SGP may include further recognition or binding sites for further factors. A subgenomic promoter is typically a genetic element of a positive strand RNA virus, such as an alphavirus. A subgenomic promoter of alphavirus is a nucleic acid sequence comprised in the viral genomic RNA. The subgenomic promoter is generally characterized in that it allows initiation of the transcription (RNA synthesis) in the presence of an RNA-dependent RNA polymerase, e.g., functional alphavirus non-structural protein. An RNA (−) strand, i.e., the complement of alphaviral genomic RNA, serves as a template for synthesis of a (+) strand subgenomic transcript, and synthesis of the (+) strand subgenomic transcript is typically initiated at or near the subgenomic promoter. The term “subgenomic promoter” as used herein, is not confined to any particular localization in a nucleic acid comprising such subgenomic promoter. In some embodiments, the SGP is identical to CSE 3 or overlaps with CSE 3 or comprises CSE 3.

The terms “subgenomic transcript” or “subgenomic RNA” synonymously refer to an RNA molecule that is obtainable as a result of transcription using a RNA molecule as template (“template RNA”), wherein the template RNA comprises a subgenomic promoter that controls transcription of the subgenomic transcript. The subgenomic transcript is obtainable in the presence of an RNA-dependent RNA polymerase, in particular functional alphavirus non-structural protein. For instance, the term “subgenomic transcript” may refer to the RNA transcript that is prepared in a cell infected by an alphavirus, using the (−) strand complement of alphavirus genomic RNA as template. However, the term “subgenomic transcript”, as used herein, is not limited thereto and also includes transcripts obtainable by using heterologous RNA as template. For example, subgenomic transcripts are also obtainable by using the (−) strand complement of SGP-containing replicons according to the present invention as template. Thus, the term “subgenomic transcript” may refer to an RNA molecule that is obtainable by transcribing a fragment of alphavirus genomic RNA, as well as to an RNA molecule that is obtainable by transcribing a fragment of a replicable RNA according to the present invention.

The term “heterologous” is used to describe something consisting of multiple different elements. As an example, the introduction of one individual's cell into a different individual constitutes a heterologous transplant. A heterologous gene is a gene derived from a source other than the subject.

Cells that may be used in the methods for identifying sequence changes are any appropriate cell in which the RNA, with or without any nucleotide modifications, can be replicated and/or translated. The cell may be a mammalian cell, for example, a human cell. The cell may constitutively express a replicase which recognizes the sequences present in a replicable RNA for replication or may transiently express such replicase.

The following provides specific and/or preferred variants of the individual features of the invention. The present invention also contemplates as particularly preferred embodiments those embodiments, which are generated by combining two or more of the specific and/or preferred variants described for two or more of the features of the present invention.

### System Comprising Two RNA Molecules

According to the invention, a system comprising two RNA molecules refers to a combination of physical entities, wherein the entities can be realized, for example, as separate compositions or as a single composition. In a preferred embodiment, the system is a composition comprising the RNA molecules and further components, such as lipids, which form particles with the RNA. It is also possible that the system is made by combining two different compositions, wherein the first composition comprises the first RNA and the second composition the second RNA. It is also possible in another embodiment that the two RNAs are present in separate compositions, each composition comprising lipids or polymers for complexing the RNAs. In this embodiment, each composition can be used separately for providing, such as by administration, the RNAs to a subject, e.g., subsequently.

In a preferred embodiment the system can comprise one or more cells, wherein the two RNA molecules are present in the same cell or can be present in different cells, preferably in the same. In a preferred embodiment, these cells can be in a subject or can be administered to a subject.

### RNAs

RNA molecules according to the invention may optionally be characterized by further features, e.g. by a 5′-cap, a 5′-UTR, a 3′-UTR, a poly(A) sequence, and/or adaptation of the codon usage for optimized translation and/or stabilization of the RNA molecule, as detailed below.

### Cap

In some embodiments, the RNA molecules according to the present invention comprises a 5′-cap.

The terms “5′-cap”, “cap”, “5′-cap structure”, “cap structure” are used synonymously to refer to a dinucleotide that is found on the 5′ end of some eukaryotic primary transcripts such as precursor messenger RNA. A 5′-cap is a structure wherein a (optionally modified) guanosine is bonded to the first nucleotide of an mRNA molecule via a 5′ to 5′ triphosphate linkage (or modified triphosphate linkage in the case of certain cap analogs). The terms can refer to a conventional cap or to a cap analog.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA molecules comprise a 5′-cap. In one embodiment, the RNA molecules do not comprise a 5′-cap. In one embodiment, only one RNA molecule, the first or second, comprises a 5′-cap.

The term “conventional 5′-cap” refers to a naturally occurring 5′-cap, preferably to the 7-methylguanosine cap. In the 7-methylguanosine cap, the guanosine of the cap is a modified guanosine wherein the modification consists of a methylation at the 7-position.

In the context of the present invention, the term “5′-cap analog” refers to a molecular structure that resembles a conventional 5′-cap, but is modified to possess the ability to stabilize RNA if attached thereto, preferably in vivo and/or in a cell. A cap analog is not a conventional 5′-cap.

For the case of eukaryotic mRNA, the 5′-cap has been generally described to be involved in efficient translation of mRNA: in general, in eukaryotes, translation is initiated only at the 5′ end of a messenger RNA (mRNA) molecule, unless an internal ribosomal entry site (IRES) is present. Eukaryotic cells are capable of providing an RNA with a 5′-cap during transcription in the nucleus: newly synthesized mRNAs are usually modified with a 5′-cap structure, e.g.; when the transcript reaches a length of 20 to 30 nucleotides. First, the 5′ terminal nucleotide pppN (ppp representing triphosphate; N representing any nucleoside) is converted in the cell to 5′ GpppN by a capping enzyme having RNA 5′-triphosphatase and guanylyltransferase activities. The GpppN may subsequently be methylated in the cell by a second enzyme with (guanine-7)-methyltransferase activity to form the mono-methylated m⁷GpppN cap. In one embodiment, the 5′-cap used in the present invention is a natural 5′-cap.

In the present invention, a natural 5′-cap dinucleotide is typically selected from the group consisting of a non-methylated cap dinucleotide (G(5′)ppp(5′) N; also termed GpppN) and a methylated cap dinucleotide ((m⁷G(5′)ppp(5′) N; also termed m⁷GpppN). m⁷GpppN (wherein N is G) is represented by the following formula:

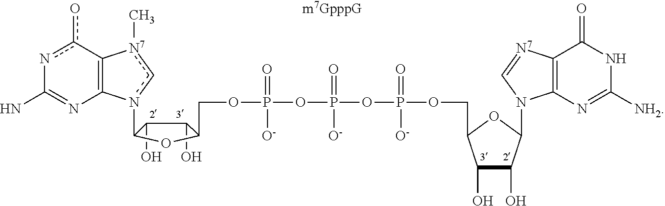

Capped RNA of the present invention can be prepared in vitro, and therefore, does not depend on a capping machinery in a host cell. The most frequently used method to make capped RNAs in vitro is to transcribe a DNA template with either a bacterial or bacteriophage RNA polymerase in the presence of all four ribonucleoside triphosphates and a cap dinucleotide such as m⁷G (5′)ppp(5′) G (also called m⁷GpppG). The RNA polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule). The formation of the competing GTP-initiated product pppGpN is suppressed by setting the molar ratio of cap to GTP between 5 and 10 during in vitro transcription.

In preferred embodiments of the present invention, the 5′-cap (if present) is a 5′-cap analog. These embodiments are particularly suitable if the RNA is obtained by in vitro transcription, e.g. is an in vitro transcribed RNA (IVT-RNA). Cap analogs have been initially described to facilitate large scale synthesis of RNA transcripts by means of in vitro transcription.

For messenger RNA, some cap analogs (synthetic caps) have been generally described to date, and they can all be used in the context of the present invention. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN). Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′—OH group of the methylated guanosine unit (Stepinski et al., 2001, RNA J. 7:1486-1495; Peng et al., 2002, Org. Lett. 24:161-164). RNAs which are synthesized in presence of such “anti reverse cap analogs” are translated more efficiently than RNAs which are in vitro transcribed in presence of the conventional 5′-cap m⁷GpppG. To that end, one cap analog in which the 3′ OH group of the methylated guanosine unit is replaced by OCH₃ is described, e.g., by Holtkamp et al., 2006, Blood 108:4009-4017 (7-methyl (3′-O-methyl) GpppG; anti-reverse cap analog (ARCA)). ARCA is a suitable cap dinucleotide according to the present invention.

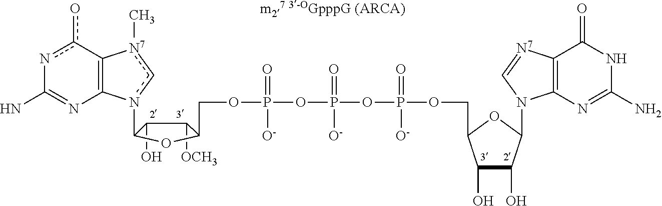

In an embodiment, the RNA of the present invention is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and B phosphate groups of the triphosphate bridge. In the present invention, a cap analog may be selected or present that is not susceptible, or less susceptible, to that type of cleavage. A suitable cap analog for this purpose may be selected from a cap dinucleotide according to formula (I):

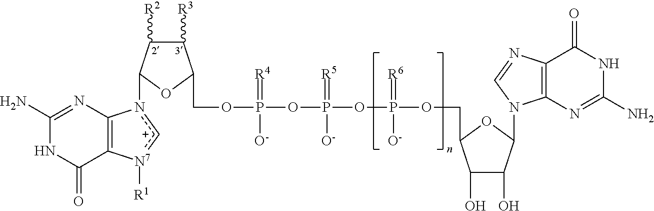

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form-O—CH₂— or —CH₂—O—,
R⁵ is selected from the group consisting of S, Se, and BH₃,
R⁴ and R⁶ are independently selected from the group consisting of O, S, Se, and BH₃.
n is 1, 2, or 3.

Preferred embodiments for R¹, R², R³, R⁴, R⁵, R⁶ are disclosed in WO 2011/015347 A1 and may be selected accordingly in the present invention.

For example, in an embodiment, the RNA of the present invention comprises a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present invention, R⁵ in Formula (I) is S; and R⁴ and R⁶ are O.

In a further embodiment, the RNA of the present invention comprises a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present invention. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for eIF4E than the corresponding ARCAs lacking a phosphorothioate group.

A respective cap analog that is particularly preferred in the present invention, i.e., m_{2′}^{7,2′-0}GppₛpG, is termed beta-S-ARCA (WO 2008/157688 A2; Kuhn et al., 2010, Gene Ther. 17:961-971). Thus, in one embodiment of the present invention, the RNA of the present invention is modified with beta-S-ARCA. beta-S-ARCA is represented by the following structure:

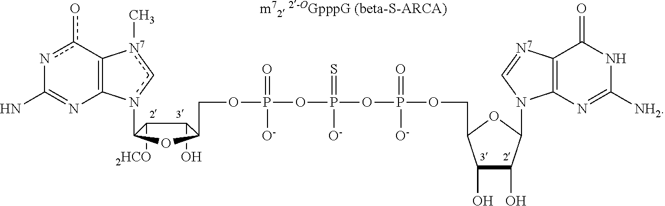

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA″ or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. Determination of the stereochemical configuration by HPLC is described in WO 2011/015347 A1.

In a first particularly preferred embodiment of the present invention, RNA of the present invention is modified with the beta-S-ARCA (D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5′-cap), whereas RNA with the beta-S-ARCA (D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA (D2) cap are more efficiently translated in mammalian cells than RNAs carrying the beta-S-ARCA (D1) cap. Therefore, in one embodiment of the present invention, RNA of the present invention is modified with a cap analog according to Formula (I), characterized by a stereochemical configuration at the P atom comprising the substituent R⁵ in Formula (I) that corresponds to that at the Pp atom of the D2 diastereomer of beta-S-ARCA. In that embodiment, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In a second particularly preferred embodiment, RNA of the present invention is modified with the beta-S-ARCA (D1) diastereomer. This embodiment is particularly suitable for transfer of capped RNA into immature antigen presenting cells, such as for vaccination purposes. It has been demonstrated that the beta-S-ARCA (D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present invention, RNA of the present invention is modified with a cap analog according to Formula (I), characterized by a stereochemical configuration at the P atom comprising the substituent R⁵ in Formula (I) that corresponds to that at the PB atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the Pp atom of the D1 diastereomer of beta-S-ARCA, may be used in the present invention. Preferably, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In one embodiment, RNA of the present invention is modified with a 5′-cap structure according to Formula (I) wherein any one phosphate group is replaced by a boranophosphate group or a phosphoroselenoate group. Such caps have increased stability both in vitro and in vivo. Optionally, the respective compound has a 2′-O- or 3′-O-alkyl group (wherein alkyl is preferably methyl); respective cap analogs are termed BH₃-ARCAs or Se-ARCAs. Compounds that are particularly suitable for capping of mRNA include the β-BH₃-ARCAs and β-Se-ARCAs, as described in WO 2009/149253 A2. For these compounds, a stereochemical configuration at the P atom comprising the substituent R⁵ in Formula (I) that corresponds to that at the PB atom of the D1 diastereomer of beta-S-ARCA is preferred.

In one embodiment, the 5′ cap can be a CleanCap supplied by Trilink Biotechnologies, San Diego, CA having the following structure:

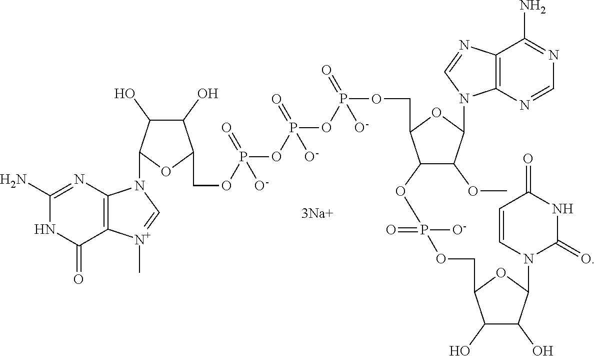 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In one embodiment, the 5′ cap can be a CleanCap supplied by Trilink Biotechnologies, San Diego, CA having the following structure:

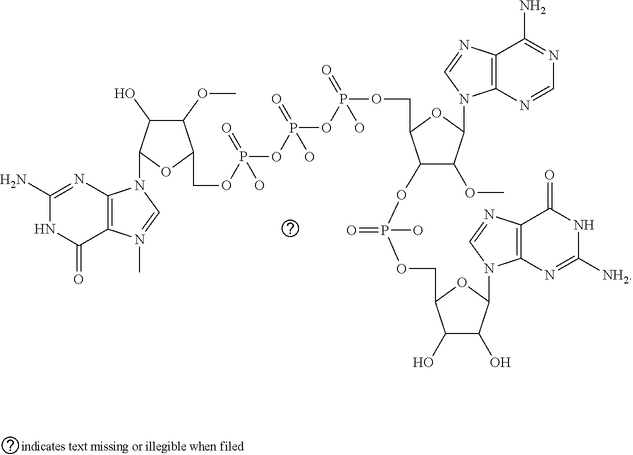

In an embodiment, a modified RNA molecule comprises a 5′-cap and wherein at least one of the uridines in the molecule is a modified uridine, preferably N1-methyl-pseudouridine (1mψ), and wherein the molecule comprises a 5′ cap having the sequence NpppNU, wherein the U in the 5′ cap is an unmodified uridine. In an embodiment, the 5′ cap has the sequence NpppAU with A representing a modified or unmodified adenosine nucleotide. For example, a modified nucleotide N or A 3′ to the triphosphate linkage may have a modified ribose structure such as a 2′-O-methylated ribose (Nm or Am) resulting in a so-called “Cap 1”. In contrast, a cap comprising a nucleotide N or A 3′ to the triphosphate linkage having an unmethylated ribose is usually referred to as “Cap 0”.

In an embodiment, the modified adenosine is selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine.

### UTR

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) sitail. Thus, the 3′-UTR is upstream of the poly(A) tail (if present), e.g. directly adjacent to the poly(A) sitail.

A 5′-UTR, if present, is located at the 5′ end of a gene, upstream of the start codon of a protein-encoding region. A 5′-UTR is downstream of the 5′-cap (if present), e.g. directly adjacent to the 5′-cap.

5′- and/or 3′-untranslated regions may, according to the invention, be functionally linked to an open reading frame, so as for these regions to be associated with the open reading frame in such a way that the stability and/or translation efficiency of the RNA comprising said open reading frame are increased.

In some embodiments, the RNA molecules according to the present invention comprise a 5′-UTR and/or a 3′-UTR. In some embodiments, the at least one miRNA sequence as described herein is located or comprised within the 3′-UTR of the second RNA molecule.

UTRs are implicated in stability and translation efficiency of RNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA molecules. Independently or additionally, it is preferable that a 3′-UTR is present that is active in order to increase the translation efficiency and/or stability of the RNA molecules.

The terms “active in order to increase the translation efficiency” and/or “active in order to increase the stability”, with reference to a first nucleic acid sequence (e.g. a UTR), means that the first nucleic acid sequence is capable of modifying, in a common transcript with a second nucleic acid sequence, the translation efficiency and/or stability of said second nucleic acid sequence in such a way that said translation efficiency and/or stability is increased in comparison with the translation efficiency and/or stability of said second nucleic acid sequence in the absence of said first nucleic acid sequence.

In one embodiment, the RNA molecules according to the present invention comprise a 5′-UTR and/or a 3′-UTR which is heterologous or non-native to the alphavirus from which the functional alphavirus replicase is derived. This allows the untranslated regions to be designed according to the desired translation efficiency and RNA stability. Thus, heterologous or non-native UTRs allow for a high degree of flexibility, and this flexibility is advantageous compared to native alphaviral UTRs.

Preferably, the RNA molecules according to the present invention comprise a 5′-UTR and/or a 3′-UTR that is not of virus origin; particularly not of alphavirus origin. In one embodiment, the RNA molecules comprise a 5′-UTR derived from a eukaryotic 5′-UTR and/or a 3′-UTR derived from a eukaryotic 3′-UTR.

A 5′-UTR according to the present invention can comprise any combination of more than one nucleic acid sequence, optionally separated by a linker. A 3′-UTR according to the present invention can comprise any combination of more than one nucleic acid sequence, optionally separated by a linker.

The term “linker” according to the invention relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3′-untranslated region of tPA is about 800 nucleotides in length, that of factor VIII is about 1800 nucleotides in length and that of erythropoietin is about 560 nucleotides in length. In some embodiments, the 3′-UTR of the second RNA molecule further comprises at least one miRNA sequence as described herein. Each miRNA sequence may be 10-200 nucleotides in length, optionally 10-100, 10-90, 10-80, 10-70, 10-60, 10-50, 10-40, 10-30, 20-100, 20-90, 20-80, 20-70, 20-60, 20-50, 20-40, or 20-30 nucleotides in length, optionally 10-50, preferably 10-30 nucleotides in length.

The 3′-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. 3′-untranslated regions may contain one or more inverted repeats which can fold to give stem-loop structures which act as barriers for exoribonucleases or interact with proteins known to increase RNA stability (e.g. RNA-binding proteins).

The human beta-globin 3′-UTR, particularly two consecutive identical copies of the human beta-globin 3′-UTR, contributes to high transcript stability and translational efficiency (Holtkamp et al., 2006, Blood 108:4009-4017). Thus, in one embodiment, the RNA molecules according to the present invention comprise two consecutive identical copies of the human beta-globin 3′-UTR. Thus, it comprises in the 5′→3′ direction: (a) optionally a 5′-UTR; (b) an open reading frame; (c) a 3′-UTR; said 3′-UTR comprising two consecutive identical copies of the human beta-globin 3′-UTR, a fragment thereof, or a variant of the human beta-globin 3′-UTR or fragment thereof.

In an embodiment, the RNA molecules according to the present invention comprise a 3′-UTR which is active in order to increase translation efficiency and/or stability, but which is not the human beta-globin 3′-UTR, a fragment thereof, or a variant of the human beta-globin 3′-UTR or fragment thereof. An exemplary human beta-globin 3′-UTR sequence is depicted in SEQ ID NO: 51. In an embodiment, a human beta-globin 3′-UTR sequence useful in the RNA molecules described herein is one that is at least 75%, 80%, 85%, 90%, 95%, 98% or 99% homologous to SEQ ID NO: 51.

In an embodiment, the RNA molecules according to the present invention comprise a 5′-UTR which is active in order to increase translation efficiency and/or stability.

In some embodiments, the RNA molecules can comprise a 3′-UTR sequence, which is a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA. These were identified by an ex vivo selection process for sequences that confer RNA stability and augment total protein expression (see WO 2017/060314, herein incorporated by reference). An exemplary FI element sequence is depicted in SEQ ID NO: 43. In an embodiment, an FI element sequence useful in the RNA molecules described herein is one that is at least 75%, 80%, 85%, 90%, 95%, 98% or 99% homologous to SEQ ID NO: 43.

### Poly(A) Sequence

In some embodiments, the first and/or second RNA molecule according to the present invention comprises a poly(A) sequence. If an RNA molecule comprises conserved sequence element 4 (CSE 4), the poly(A) sequence of the RNA molecule is preferably present downstream of CSE 4, most preferably directly adjacent to CSE 4. In some embodiments, the poly(A) sequence is a 3′ poly(A) sequence.

According to the invention, in one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150, A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e. 100% by number of nucleotides in the poly(A) sequence, are A nucleotides. The term “A nucleotide” or “A” refers to adenylate.

Indeed, it has been demonstrated that a 3′ poly(A) sequence of about 120 A nucleotides has a beneficial influence on the levels of RNA in transfected eukaryotic cells, as well as on the levels of protein that is translated from an open reading frame that is present upstream (5′) of the 3′ poly(A) sequence (Holtkamp et al., 2006, Blood, vol. 108, pp. 4009-4017).

In alphaviruses, a 3′ poly(A) sequence of at least 11 consecutive adenylate residues, or at least 25 consecutive adenylate residues, is thought to be important for efficient synthesis of the minus strand. In particular, in alphaviruses, a 3′ poly(A) sequence of at least 25 consecutive adenylate residues is understood to function together with conserved sequence element 4 (CSE 4) to promote synthesis of the (−) strand (Hardy & Rice, 2005, J. Virol. 79:4630-4639).

The present invention provides for a 3′ poly(A) sequence to be attached during RNA transcription, i.e. during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

The first and/or second RNA molecule may comprise an interrupted 3′ poly(A) sequence. In a preferred embodiment of the present invention, the 3′ poly(A) cassette present in the coding strand of DNA essentially consists of dA nucleotides, but is interrupted by a random sequence having an equal distribution of the four nucleotides (dA, dC, dG, dT). Such random sequence may be 5 to 50, preferably 10 to 30, more preferably 10 to 20 nucleotides in length. Such a cassette is disclosed in WO 2016/005004 A1. Any poly(A) cassette disclosed in WO 2016/005004 A1 may be used in the present invention. A poly(A) cassette that essentially consists of dA nucleotides, but is interrupted by a random sequence having an equal distribution of the four nucleotides (dA, dC, dG, dT) and having a length of, e.g., 5 to 50 nucleotides shows, on DNA level, constant propagation of plasmid DNA in E. coli and is still associated, on RNA level, with the beneficial properties with respect to supporting RNA stability and translational efficiency.

Consequently, in a preferred embodiment of the present invention, the 3′ poly(A) sequence contained in an RNA molecule described herein essentially consists of A nucleotides, but is interrupted by a random sequence having an equal distribution of the four nucleotides (A, C, G, U). Such random sequence may be 5 to 50, preferably 10 to 30, more preferably 10 to 20 nucleotides in length. In some embodiments, the first and/or second RNA molecule comprise an interrupted 3′ poly(A) sequence which consists of A30-L-A70, wherein the linker (L) is 10 nucleotides in length.

### Codon Usage

In general, the degeneracy of the genetic code will allow the substitution of certain codons (base triplets coding for an amino acid) that are present in an RNA sequence by other codons (base triplets), while maintaining the same coding capacity (so that the replacing codon encodes the same amino acid as the replaced codon). In some embodiments of the present invention, at least one codon of an open reading frame comprised by an RNA molecule differs from the respective codon in the respective open reading frame in the species from which the open reading frame originates. In that embodiment, the coding sequence of the open reading frame is said to be “adapted” or “modified”. The coding sequence of an open reading frame comprised by the first and/or second RNA may be adapted.

For example, when the coding sequence of an open reading frame is adapted, frequently used codons may be selected: WO 2009/024567 A1 describes the adaptation of a coding sequence of a nucleic acid molecule, involving the substitution of rare codons by more frequently used codons. Since the frequency of codon usage depends on the host cell or host organism, that type of adaptation is suitable to fit a nucleic acid sequence to expression in a particular host cell or host organism. Generally, speaking, more frequently used codons are typically translated more efficiently in a host cell or host organism, although adaptation of all codons of an open reading frame is not always required.

For example, when the coding sequence of an open reading frame is adapted, the content of G (guanylate) residues and C (cytidylate) residues may be altered by selecting codons with the highest GC-rich content for each amino acid. RNA molecules with GC-rich open reading frames were reported to have the potential to reduce immune activation and to improve translation and half-life of RNA (Thess et al., 2015, Mol. Ther. 23:1457-1465).

In particular, the coding sequence for non-structural protein can be adapted as desired. This freedom is possible because the open reading frame encoding non-structural protein does not overlap with the 5′ replication recognition sequence of the replicon.

### RNA Modifications

In an embodiment, the first and/or second RNA described herein may have modified nucleotides/nucleosides/backbone modifications. The term “RNA modification” as used herein may refer to chemical modifications comprising backbone modifications as well as sugar modifications or base modifications.

In this context, a modified RNA molecule as defined herein may contain nucleotide analogues/modifications, e.g., backbone modifications, sugar modifications or base modifications. A backbone modification in connection with the present invention is a modification, in which phosphates of the backbone of the nucleotides contained in an RNA molecule as defined herein are chemically modified. A sugar modification in connection with the present invention is a chemical modification of the sugar of the nucleotides of the RNA molecule as defined herein. Furthermore, a base modification in connection with the present invention is a chemical modification of the base moiety of the nucleotides of the RNA molecule. In this context, nucleotide analogues or modifications are preferably selected from nucleotide analogues, which are applicable for transcription and/or translation.

Sugar Modifications: The modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), -0(CH2CH20)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be incorporated into a modified RNA molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present invention, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-tri-phosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, N6-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-I-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, I-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O-(I-thiophosphate)-adenosine, 5′-O-(1-thiophosphate)-cytidine, 5′-O-(I-thiophosphate)-guanosine, 5′-O-(I-thiophosphate)-uridine or 5′-O-(I-thiophosphate)-pseudouridine.

In further embodiments, a modified RNA may comprise nucleoside modifications selected from 6-aza-cytidine, 2-thio-cytidine, a-thio-cytidine, pseudo-iso-cytidine, 5-aminoallyl-uridine, 5-iodo-uridine, NI-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytdine, 8-oxo-guanosine, 7-deaza-guanosine, NI-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the RNA comprises a modified nucleoside in place of at least one (e.g., every) uridine.

The term “uracil,” as used herein, describes one of the nucleobases that can occur in the nucleic acid of RNA. The structure of uracil is:

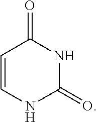

The term “uridine,” as used herein, describes one of the nucleosides that can occur in RNA. The structure of uridine is:

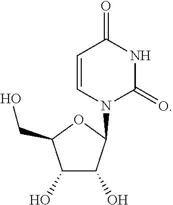 [SMILES: O=c1ccn([C@@H]2O[C@H](CO)[C@@H](O)[C@H]2O)c(=O)[nH]1]

UTP (uridine 5′-triphosphate) has the following structure:

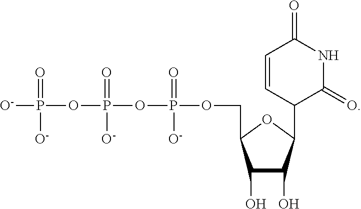

Pseudo-UTP (pseudouridine 5′-triphosphate) has the following structure:

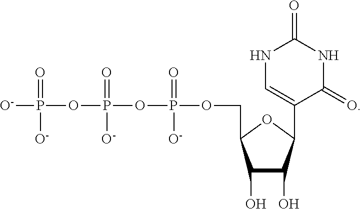

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

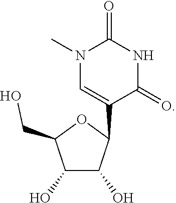 [SMILES: Cn1cc([C@@H]2O[C@H](CO)[C@@H](O)[C@H]2O)c(=O)[nH]c1=O]

N1-methyl-pseudo-UTP has the following structure:

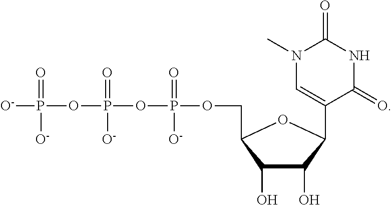

Another exemplary modified nucleoside is 5-methyl-uridine (m5U), which has the structure:

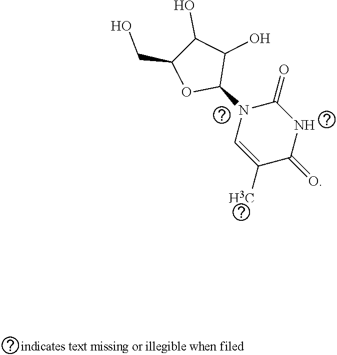 [SMILES: [3CH3]c1cn([C@H]2O[C@@H](CO)C(O)C2O)c(=O)[nH]c1=O]

In certain preferred embodiments, one or more uridine in the RNA described herein is replaced by a modified nucleoside. In some embodiments, the modified nucleoside is a modified uridine.

In certain preferred embodiments, RNA comprises a modified nucleoside in place of at least one uridine. In some embodiments, RNA comprises a modified nucleoside in place of each uridine.

In certain preferred embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In certain preferred embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s²U), 5-aminomethyl-2-thio-uridine (nm5s²U), 5-methylaminomethyl-uridine (mnm5U), 1-ethyl-pseudouridine, 5-methylaminomethyl-2-thio-uridine (mnm5s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s²U), 1-taurinomethyl-4-thio-pseudouridine), 5-methyl-2-thio-uridine (m⁵s²U), 1-methyl-4-thio-pseudouridine (m1s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ w), 5-(isopentenylaminomethyl) uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art. In some embodiments, the first and the second RNA molecule comprise a modified nucleoside in place of at least one uridine, preferably in place of each uridine; preferably wherein the modified nucleoside is independently selected from pseudouridine (ψ), N1-methyl-pseudouridine (m1ψ), and 5-methyl-uridine (m5U). In some embodiments, the first RNA molecule, but not the second RNA molecule, comprises a modified nucleoside in place of at least one uridine, preferably in place of each uridine; preferably wherein the modified nucleoside is independently selected from pseudouridine (ψ), N1-methyl-pseudouridine (m1ψ), and 5-methyl-uridine (m5U). In some embodiments, the second RNA molecule, but not the first RNA molecule, comprises a modified nucleoside in place of at least one uridine, preferably in place of each uridine; preferably wherein the modified nucleoside is independently selected from pseudouridine (ψ), N1-methyl-pseudouridine (m1ψ), and 5-methyl-uridine (m5U).

In an embodiment, the RNA comprises other modified nucleosides or comprises further modified nucleosides, e.g., modified cytidine such as those described above. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In an embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

### First RNA Molecule

The first RNA molecule comprises an open reading frame encoding a functional RNA-dependent RNA polymerase (replicase). In an embodiment, the first RNA molecule is a replicon, which can be replicated by its encoded replicase. In this embodiment, the first RNA molecule comprises nucleotide sequences that can be recognized by the replicase such that the RNA is replicated. The first RNA molecule can further comprise other features.

In an embodiment, the first RNA molecule cannot be replicated by its encoded replicase, preferably cannot be replicated by any replicase from a self-replicating virus. In this embodiment, the first RNA molecule may lack sequences usually required for replication as described herein.

In an embodiment, the first RNA is an mRNA and preferably comprises further features of typical eukaryotic mRNAs, such as 5′cap or poly(A) tail, as described herein.

In an embodiment, the first RNA molecule comprises an open reading frame encoding a functional replicase and a further open reading frame encoding a protein of interest.

### Functional Replicase

The term “non-structural protein” relates to a protein encoded by a virus but that is not part of the viral particle. This term typically includes the various enzymes and transcription factors the virus uses to replicate itself, such as RNA replicase or other template-directed polymerases. The term “non-structural protein” includes each and every co- or post-translationally modified form, including carbohydrate-modified (such as glycosylated) and lipid-modified forms of a non-structural protein and preferably relates to an “alphavirus non-structural protein”.

In some embodiments, the term “alphavirus non-structural protein” refers to any one or more of individual non-structural proteins of alphavirus origin (nsP1, nsP2, nsP3, nsP4), or to a poly-protein comprising the polypeptide sequence of more than one non-structural protein of alphavirus origin. In some embodiments, “alphavirus non-structural protein” refers to nsP123 and/or to nsP4. In other embodiments, “alphavirus non-structural protein” refers to nsP1234. In one embodiment, the protein of interest encoded by an open reading frame consists of all of nsP1, nsP2, nsP3 and nsP4 as one single, optionally cleavable poly-protein: nsP1234. In one embodiment, the protein of interest encoded by an open reading frame consists of nsP1, nsP2 and nsP3 as one single, optionally cleavable polyprotein: nsP123. In that embodiment, nsP4 may be a further protein of interest and may be encoded by a further open reading frame.

In some embodiments, non-structural protein is capable of forming a complex or association, e.g., in a host cell. In some embodiments, “alphavirus non-structural protein” refers to a complex or association of nsP123 (synonymously P123) and nsP4. In some embodiments, “alphavirus non-structural protein” refers to a complex or association of nsP1, nsP2, and nsP3. In some embodiments, “alphavirus non-structural protein” refers to a complex or association of nsP1, nsP2, nsP3 and nsP4. In some embodiments, “alphavirus non-structural protein” refers to a complex or association of any one or more selected from the group consisting of nsP1, nsP2, nsP3 and nsP4. In some embodiments, the alphavirus non-structural protein comprises at least nsP4.

The terms “complex” or “association” refer to two or more same or different protein molecules that are in spatial proximity. Proteins of a complex are preferably in direct or indirect physical or physicochemical contact with each other. A complex or association can consist of multiple different proteins (heteromultimer) and/or of multiple copies of one particular protein (homomultimer). In the context of alphavirus non-structural protein, the term “complex or association” describes a multitude of at least two protein molecules, of which at least one is an alphavirus non-structural protein. The complex or association can consist of multiple copies of one particular protein (homomultimer) and/or of multiple different proteins (heteromultimer). In the context of a multimer, “multi” means more than one, such as two, three, four, five, six, seven, eight, nine, ten, or more than ten.

The term “functional non-structural protein” includes non-structural protein that has replicase function. Thus, “functional non-structural protein” includes alphavirus replicase. “Replicase function” comprises the function of an RNA-dependent RNA polymerase (RdRP), i.e., an enzyme which is capable to catalyze the synthesis of (−) strand RNA based on a (+) strand RNA template, and/or which is capable to catalyze the synthesis of (+) strand RNA based on a (−) strand RNA template. Thus, the term “functional non-structural protein” can refer to a protein or complex that synthesizes (−) stranded RNA, using the (+) stranded (e.g. genomic) RNA as template, to a protein or complex that synthesizes new (+) stranded RNA, using the (−) stranded complement of genomic RNA as template, and/or to a protein or complex that synthesizes a subgenomic transcript, using a fragment of the (−) stranded complement of genomic RNA as template. The functional non-structural protein may additionally have one or more additional functions, such as, e.g., a protease (for auto-cleavage), helicase, terminal adenylyltransferase (for poly(A) tail addition), methyltransferase and guanylyltransferase (for providing a nucleic acid with a 5′-cap), nuclear localization sites, triphosphatase (Gould et al., 2010, Antiviral Res. 87:111-124; Rupp et al., 2015, J. Gen. Virol. 96:2483-500).

In some embodiments, the term “functional non-structural protein” is a synonym for “functional replicase”.

The term “replicase” includes RNA-dependent RNA polymerase. According to the invention, the term “replicase” includes “alphavirus replicase”, including a RNA-dependent RNA polymerase from a naturally occurring alphavirus (alphavirus found in nature) and a RNA-dependent RNA polymerase from a variant or derivative of an alphavirus, such as from an attenuated alphavirus. The term “replicase” may also include a RNA-dependent RNA polymerase from other self-replicating viruses, such as a self-replicating single-stranded RNA virus, optionally a positive-sense, single-stranded RNA virus (e.g., alphavirus, flavivirus, etc.).

The term “replicase” comprises all variants, in particular post-translationally modified variants, conformations, isoforms and homologs of alphavirus replicase, which are expressed by alphavirus-infected cells or which are expressed by cells that have been transfected with a nucleic acid that codes for alphavirus replicase. Moreover, the term “replicase” comprises all forms of replicase that have been produced and can be produced by recombinant methods. For example, a replicase comprising a tag that facilitates detection and/or purification of the replicase in the laboratory, e.g.; a myc-tag, a HA-tag or an oligohistidine tag (His-tag) may be produced by recombinant methods.

Optionally, the alphavirus replicase is additionally functionally defined by the capacity of binding to any one or more of alphavirus conserved sequence element 1 (CSE 1) or complementary sequence thereof, conserved sequence element 2 (CSE 2) or complementary sequence thereof, conserved sequence element 3 (CSE 3) or complementary sequence thereof, conserved sequence element 4 (CSE 4) or complementary sequence thereof. Preferably, the replicase is capable of binding to CSE 2 [i.e., to the (+) strand] and/or to CSE 4 [i.e., to the (+) strand], or of binding to the complement of CSE 1 [i.e. to the (−) strand] and/or to the complement of CSE 3 [i.e., to the (−) strand].

The origin of the alphavirus replicase is not limited to any particular alphavirus. In a preferred embodiment, the alphavirus replicase comprises non-structural protein from Semliki Forest virus, including a naturally occurring Semliki Forest virus and a variant or derivative of Semliki Forest virus, such as an attenuated Semliki Forest virus. In an alternative preferred embodiment, the alphavirus replicase comprises non-structural protein from Sindbis virus, including a naturally occurring Sindbis virus and a variant or derivative of Sindbis virus, such as an attenuated Sindbis virus. In an alternative preferred embodiment, the alphavirus replicase comprises non-structural protein from Venezuelan equine encephalitis virus (VEEV), including a naturally occurring VEEV and a variant or derivative of VEEV, such as an attenuated VEEV. In an alternative preferred embodiment, the alphavirus replicase comprises non-structural protein from chikungunya virus (CHIKV), including a naturally occurring CHIKV and a variant or derivative of CHIKV, such as an attenuated CHIKV.

A replicase can also comprise non-structural proteins from more than one virus, e.g., from more than one alphavirus. Thus, heterologous complexes or associations comprising alphavirus non-structural protein and having replicase function are equally comprised by the present invention. Merely for illustrative purposes, replicase may comprise one or more non-structural proteins (e.g., nsP1, nsP2) from a first alphavirus, and one or more non-structural proteins (nsP3, nsP4) from a second alphavirus. Non-structural proteins from more than one different alphavirus may be encoded by separate open reading frames, or may be encoded by a single open reading frame as poly-protein, e.g., nsP1234.

In some embodiments, functional non-structural protein is capable of forming membranous replication complexes and/or vacuoles in cells in which the functional non-structural protein is expressed.

If functional non-structural protein, i.e., non-structural protein with replicase function, is encoded by a nucleic acid molecule according to the present invention, it is preferable that the subgenomic promoter of the replicon, if present, is compatible with said replicase. Compatible in this context means that the replicase is capable of recognizing the subgenomic promoter, if present. In one embodiment, this is achieved when the subgenomic promoter is native to the virus from which the replicase is derived, i.e. the natural origin of these sequences is the same virus. In an alternative embodiment, the subgenomic promoter is not native to the virus from which the virus replicase is derived, provided that the virus replicase is capable of recognizing the subgenomic promoter. In other words, the replicase is compatible with the subgenomic promoter (cross-virus compatibility). Examples of cross-virus compatibility concerning subgenomic promoter and replicase originating from different alphaviruses are known in the art. Any combination of subgenomic promoter and replicase is possible as long as cross-virus compatibility exists. Cross-virus compatibility can readily be tested by the skilled person working the present invention by incubating a replicase to be tested together with an RNA, wherein the RNA has a subgenomic promoter to be tested, at conditions suitable for RNA synthesis from the a subgenomic promoter. If a subgenomic transcript is prepared, the subgenomic promoter and the replicase are determined to be compatible. Various examples of cross-virus compatibility are known.

The replicon can preferably be replicated by the functional non-structural protein. In particular, the RNA replicon that encodes functional non-structural protein can be replicated by the functional non-structural protein encoded by the replicon. In a preferred embodiment, the second RNA molecule comprises an miRNA and an open reading frame encoding a protein of interest. This embodiment is particularly suitable in some methods for producing a protein of interest according to the present invention together with a miRNA. The further open reading frame encoding a protein of interest is preferably located downstream from the 5′ replication recognition sequence and upstream from the miRNA. In an embodiment, the further open reading frame is located downstream from the miRNA. In one embodiment, the second RNA molecule comprises one or more open reading frames encoding one or more proteins of interest.

The one or more further open reading frames encoding one or more proteins of interest are generally controlled by (a) subgenomic promoter(s).

### Replicable RNAs

A replicable RNA molecule or replicable RNA (rRNA) is an RNA that can be replicated by an RNA-dependent RNA polymerase (replicase) by virtue of comprising nucleotide sequences that can be recognized by the replicase such that the RNA is replicated. The replication of the rRNA yields-without DNA intermediate-one or multiple identical or essentially identical copies of the rRNA. “Without DNA intermediate” means that no deoxyribonucleic acid (DNA) copy or complement of the rRNA is formed in the process of forming the copies of the rRNA, and/or that no deoxyribonucleic acid (DNA) molecule is used as a template in the process of forming the copies of the rRNA, or complement thereof. The replicase function is typically provided by functional non-structural proteins, e.g., functional alphavirus non-structural proteins.

According to the invention at least the second RNA molecule is a replicable RNA molecule. The second RNA molecule according to the invention is preferably replicated in trans, e.g. by a replicase not encoded on the second RNA molecule, but by a functional replicase encoded on the first RNA molecule. Preferably, the second RNA molecule does not comprise a functional replicase. The first RNA molecule may also be a replicable RNA molecule. Preferably any further RNA molecule, e.g., a third RNA molecule, is a replicable RNA molecule.

The terms “RNA replicon”, “replicon”, “replicable RNA molecule” and “replicable RNA” can be used interchangeably.

According to the invention, the terms “can be replicated” and “capable of being replicated” generally describe that one or more identical or essentially identical copies of a nucleic acid can be prepared. When used together with the term “replicase”, such as in “capable of being replicated by a replicase”, the terms “can be replicated” and “capable of being replicated” describe functional characteristics of a nucleic acid molecule, e.g. a RNA replicon, with respect to a replicase. These functional characteristics comprise at least one of (i) the replicase is capable of recognizing the replicon and (ii) the replicase is capable of acting as RNA-dependent RNA polymerase (RdRP). Preferably, the replicase is capable of both (i) recognizing the replicon and (ii) acting as RNA-dependent RNA polymerase. In a preferred embodiment, the term “can be replicated” means that the RNA contains sequences that can be recognized or bound by a functional replicase, such as any one or more of a conserved sequence element 1 (CSE 1) or complementary sequence thereof, conserved sequence element 2 (CSE 2) or complementary sequence thereof, conserved sequence element 3 (CSE 3) or complementary sequence thereof and/or conserved sequence element 4 (CSE 4) or complementary sequence thereof.

The expression “capable of recognizing” describes that the replicase is capable of physically associating with the replicon, and preferably, that the replicase is capable of binding to the replicon, typically non-covalently. The term “binding” can mean that the replicase has the capacity of binding to any one or more of a conserved sequence element 1 (CSE 1) or complementary sequence thereof (if comprised by the replicon), conserved sequence element 2 (CSE 2) or complementary sequence thereof (if comprised by the replicon), conserved sequence element 3 (CSE 3) or complementary sequence thereof (if comprised by the replicon), conserved sequence element 4 (CSE 4) or complementary sequence thereof (if comprised by the replicon). Preferably, the replicase is capable of binding to CSE 2 [i.e., to the (+) strand] and/or to CSE 4 [i.e., to the (+) strand], or of binding to the complement of CSE 1 [i.e. to the (−) strand] and/or to the complement of CSE 3 [i.e., to the (−) strand].

In one embodiment, the expression “capable of acting as RdRP” means that the replicase is capable to catalyze the synthesis of the (−) strand complement of viral genomic (+) strand RNA, wherein the (+) strand RNA has template function, and/or that the replicase is capable to catalyze the synthesis of (+) strand viral genomic RNA, wherein the (−) strand RNA has template function. In general, the expression “capable of acting as RdRP” can also include that the replicase is capable to catalyze the synthesis of a (+) strand subgenomic transcript wherein a (−) strand RNA has template function, and wherein synthesis of the (+) strand subgenomic transcript is typically initiated at a subgenomic promoter. In one embodiment, the virus is an alphavirus.

The expressions “capable of binding” and “capable of acting as RdRP” refer to the capability at normal physiological conditions. In particular, they refer to the conditions inside a cell, which expresses non-structural proteins or which has been transfected with a nucleic acid that codes for functional non-structural proteins. The cell is preferably a eukaryotic cell. The capability of binding and/or the capability of acting as RdRP can be experimentally tested, e.g. in a cell-free in vitro system or in a eukaryotic cell. Optionally, said eukaryotic cell is a cell from a species to which the particular virus that represents the origin of the replicase is infectious. For example, when the virus replicase from a particular virus is used that is infectious to humans, the normal physiological conditions are conditions in a human cell. More preferably, the eukaryotic cell (in one example human cell) is from the same tissue or organ to which the particular virus that represents the origin of the replicase is infectious.

### Uncoupling of Sequence Elements Required for Replication and Protein-Coding Regions

In an embodiment, the first and/or second replicable RNAs (rRNAs) comprise a modified regulatory region of a self-replicating single-stranded positive-sense virus comprising sequence changes compared to a reference modified regulatory region, which sequence changes restore or improve the function of the rRNA molecule that comprises at least one modified nucleotide. These changes may be identified by the methods described herein for identifying such sequence changes. In an embodiment, the modified regulatory region is an alphavirus regulatory region, e.g., a 5′ or a 3′ regulatory region. In an embodiment, the 5′ regulatory region is the VEEV alphaviral 5′ regulatory region.

Versatile alphavirus-derived vectors are difficult to develop because the open reading frame encoding nsP1234 overlaps with the 5′ replication recognition sequence of the alphavirus genome (coding sequence for nsP1) and typically also with the subgenomic promoter comprising CSE 3 (coding sequence for nsP4).

The rRNAs described herein generally comprise sequence elements required for replication by a replicase, in particular a 5′ replication recognition sequence. In an embodiment, the coding sequence for one or more non-structural proteins is under the control of an IRES and thus an IRES is located upstream of the coding sequence for non-structural proteins. Thus, in one embodiment, the 5′ replication recognition sequence which normally overlaps with the coding sequence for the N-terminal fragment of the alphavirus non-structural protein, is located upstream of the IRES and does not overlap with the coding sequence for one or more non-structural proteins.

In an embodiment, coding sequences of the 5′ replication recognition sequence such as nsP1 coding sequences are fused in frame to a gene of interest which is located upstream from the IRES.

In an embodiment, the 5′ replication recognition sequence does not encode any protein or fragment thereof, such as an alphavirus non-structural protein or fragment thereof. Thus, in rRNAs according to the invention, the sequence elements required for replication by the replicase and protein-coding regions may be uncoupled. The uncoupling may be achieved by the removal of at least one initiation codon in the 5′ replication recognition sequence compared to a native virus genomic RNA, e.g., native alphavirus genomic RNA.

Thus, the rRNAs may comprise a 5′ replication recognition sequence, wherein the 5′ replication recognition sequence is characterized in that it comprises the removal of at least one initiation codon compared to a native virus 5′ replication recognition sequence, e.g., native alphavirus 5′ replication recognition sequence.

The 5′ replication recognition sequence that is characterized in that it comprises the removal of at least one initiation codon compared to a native virus 5′ replication recognition sequence can be referred to herein as “modified 5′ replication recognition sequence” or “5′ replication recognition sequence according to the invention”. As described herein below, the 5′ replication recognition sequence according to the invention may optionally be characterized by the presence of one or more additional nucleotide changes, such as those detected by the methods of the present invention.

In one embodiment, the rRNA comprises a 3′ replication recognition sequence. A 3′ replication recognition sequence is a nucleic acid sequence that can be recognized by a functional replicase. In other words, functional replicase is capable of recognizing the 3′ replication recognition sequence. Preferably, the 3′ replication recognition sequence is located at the 3′ end of the replicon (if the replicon does not comprise a poly(A) tail), or immediately upstream of the poly(A) tail (if the replicon comprises a poly(A) tail). In one embodiment, the 3′ replication recognition sequence consists of or comprises CSE 4.

In one embodiment, the 5′ replication recognition sequence and the 3′ replication recognition sequence are capable of directing replication of the rRNA according to the present invention in the presence of functional replicase. Thus, when present alone or preferably together, these recognition sequences direct replication of the rRNA in the presence of functional replicase.

It is preferable that a functional replicase is provided by the first rRNA that is capable of recognizing both the 5′ replication recognition sequence and the 3′ replication recognition sequence of each rRNA. In one embodiment, this is achieved when the 3′ replication recognition sequence is native to the alphavirus from which the functional alphavirus replicase is derived, and when the 5′ replication recognition sequence is native to the alphavirus from which the functional alphavirus replicase is derived or is a variant of the 5′ replication recognition sequence that is native to the alphavirus from which the functional alphavirus replicase is derived. Native means that the natural origin of these sequences is the same alphavirus. In an alternative embodiment, the 5′ replication recognition sequence and/or the 3′ replication recognition sequence are not native to the alphavirus from which the functional alphavirus replicase is derived, provided that the functional alphavirus replicase is capable of recognizing both the 5′ replication recognition sequence and the 3′ replication recognition sequence of each rRNA. In other words, the functional alphavirus replicase is compatible to the 5′ replication recognition sequence and the 3′ replication recognition sequence. When a non-native functional alphavirus replicase is capable of recognizing a respective sequence or sequence element, the functional alphavirus replicase is said to be compatible (cross-virus compatibility). Any combination of (3′/5′) replication recognition sequences and CSEs, respectively, with functional alphavirus replicase is possible as long as cross-virus compatibility exists. Cross-virus compatibility can readily be tested by the skilled person working the present invention by incubating a functional alphavirus replicase to be tested together with an RNA, wherein the RNA has 3′- and 5′ replication recognition sequences to be tested, at conditions suitable for RNA replication, e.g. in a suitable host cell. If replication occurs, the (3′/5′) replication recognition sequences and the functional alphavirus replicase are determined to be compatible. In some instances, the replicase may be derived from a self-replicating single-stranded RNA virus, such as a positive-sense, single-stranded RNA virus (e.g., alphavirus, flavivirus, etc.), in which case, the 5′ replication recognition sequence and the 3′ replication recognition sequence of each rRNA may also be derived from the same self-replicating single-stranded RNA virus, such as the same positive-sense, single-stranded RNA virus (e.g., alphavirus, flavivirus, etc.).

The removal of at least one initiation codon within the 5′ replication recognition sequence provides several advantages. Absence of an initiation codon in the nucleic acid sequence encoding nsP1* (N-terminal fragment of nsP1) will typically cause that nsP1* is not translated. Further, since nsP1* is not translated, the open reading frame encoding the protein of interest (“GOI 2”) is the most upstream open reading frame accessible to the ribosome; thus, when the rRNA is present in a cell, translation is initiated at the first AUG of the open reading frame (RNA) encoding the protein of interest.

The removal of at least one initiation codon can be achieved by any suitable method known in the art. For example, a suitable DNA molecule encoding an rRNA, i.e., characterized by the removal of an initiation codon, can be designed in silico, and subsequently synthesized in vitro (gene synthesis); alternatively, a suitable DNA molecule may be obtained by site-directed mutagenesis of a DNA sequence encoding an rRNA. In any case, the respective DNA molecule may serve as template for in vitro transcription, thereby providing an rRNA according to the invention.

The removal of at least one initiation codon compared to a native 5′ replication recognition sequence is not particularly limited and may be selected from any nucleotide modification, including substitution of one or more nucleotides (including, on DNA level, a substitution of A and/or T and/or G of the initiation codon); deletion of one or more nucleotides (including, on DNA level, a deletion of A and/or T and/or G of the initiation codon), and insertion of one or more nucleotides (including, on DNA level, an insertion of one or more nucleotides between A and T and/or between T and G of the initiation codon). Irrespective of whether the nucleotide modification is a substitution, an insertion or a deletion, the nucleotide modification must not result in the formation of a new initiation codon (as an illustrative example: an insertion, at DNA level, must not be an insertion of an ATG).

The 5′ replication recognition sequence of an rRNA that is characterized by the removal of at least one initiation codon (i.e. the modified 5′ replication recognition sequence according to the present invention) is preferably a variant of a 5′ replication recognition sequence of the genome of an alphavirus found in nature. In one embodiment, the modified 5′ replication recognition sequence according to the present invention is preferably characterized by a degree of sequence identity of 80% or more, preferably 85% or more, more preferably 90% or more, even more preferably 95% or more, to the 5′ replication recognition sequence of the genome of at least one alphavirus found in nature.

In one embodiment, the 5′ replication recognition sequence of an rRNA that may be characterized by the removal of at least one initiation codon comprises a sequence homologous to about 250 nucleotides at the 5′ end of an alphavirus, i.e. at the 5′ end of the alphaviral genome. In a preferred embodiment, it comprises a sequence homologous to about 250 to 500, preferably about 300 to 500 nucleotides at the 5′ end of an alphavirus, i.e., at the 5′ end of the alphaviral genome. “At the 5′ end of the alphaviral genome” means a nucleic acid sequence beginning at, and including, the most upstream nucleotide of the alphaviral genome. In other words, the most upstream nucleotide of the alphaviral genome is designated nucleotide no. 1, and, e.g., “250 nucleotides at the 5′ end of the alphaviral genome” means nucleotides 1 to 250 of the alphaviral genome. In one embodiment, the 5′ replication recognition sequence of an rRNA is characterized by a degree of sequence identity of 80% or more, preferably 85% or more, more preferably 90% or more, even more preferably 95% or more, to at least 250 nucleotides at the 5′ end of the genome of at least one alphavirus found in nature. At least 250 nucleotides includes, e.g., 250 nucleotides, 300 nucleotides, 400 nucleotides, 500 nucleotides.

The 5′ replication recognition sequence of an alphavirus found in nature is typically characterized by at least one initiation codon and/or by conserved secondary structural motifs. For example, the native 5′ replication recognition sequence of Semliki Forest virus (SFV) comprises five specific AUG base triplets. According to Frolov et al., 2001, RNA 7:1638-1651, analysis by MFOLD revealed that the native 5′ replication recognition sequence of Semliki Forest virus is predicted to form four stem loops (SL), termed stem loops 1 to 4 (SL1, SL2, SL3, SL4). According to Frolov et al., analysis by MFOLD revealed that also the native 5′ replication recognition sequence of a different alphavirus, Sindbis virus, is predicted to form four stem loops: SL1, SL2, SL3, SL4.

It is known that the 5′ end of the alphaviral genome comprises sequence elements that enable replication of the alphaviral genome by functional alphavirus replicase. In one embodiment of the present invention, the 5′ replication recognition sequence of an rRNA comprises a sequence homologous to conserved sequence element 1 (CSE 1) and/or a sequence homologous to conserved sequence element 2 (CSE 2) of an alphavirus.

Conserved sequence element 2 (CSE 2) of alphavirus genomic RNA typically is represented by SL3 and SL4 which is preceded by SL2 comprising at least the native initiation codon that encodes the first amino acid residue of alphavirus non-structural protein nsP1. In this description, however, in some embodiments, the conserved sequence element 2 (CSE 2) of alphavirus genomic RNA refers to a region spanning from SL2 to SL4 and comprising the native initiation codon that encodes the first amino acid residue of alphavirus non-structural protein nsP1. In a preferred embodiment, an rRNA according to the invention comprises CSE 2 or a sequence homologous to CSE 2. In one embodiment, an rRNA according to the invention comprises a sequence homologous to CSE 2 that is preferably characterized by a degree of sequence identity of 80% or more, preferably 85% or more, more preferably 90% or more, even more preferably 95% or more, to the sequence of CSE 2 of at least one alphavirus found in nature.

In an embodiment, the 5′ replication recognition sequence comprises a sequence that is homologous to CSE 2 of an alphavirus. The CSE 2 of an alphavirus may comprise a fragment of an open reading frame of a non-structural protein from an alphavirus.

Thus, in an embodiment, an rRNA according to the invention is characterized in that it comprises a sequence homologous to an open reading frame of a non-structural protein or a fragment thereof from an alphavirus. The sequence homologous to an open reading frame of a non-structural protein or a fragment thereof is typically a variant of an open reading frame of a non-structural protein or a fragment thereof of an alphavirus found in nature. In one embodiment, the sequence homologous to an open reading frame of a non-structural protein or a fragment thereof is preferably characterized by a degree of sequence identity of 80% or more, preferably 85% or more, more preferably 90% or more, even more preferably 95% or more, to an open reading frame of a non-structural protein or a fragment thereof of at least one alphavirus found in nature.

In an embodiment, the sequence homologous to an open reading frame of a non-structural protein that is comprised by an rRNA of the present invention does not comprise the native initiation codon of a non-structural protein, and more preferably does not comprise any initiation codon of a non-structural protein. In an embodiment, the sequence homologous to CSE 2 is characterized by the removal of all initiation codons compared to a native alphavirus CSE 2 sequence. Thus, the sequence homologous to CSE 2 does preferably not comprise any initiation codon.

When the sequence homologous to an open reading frame does not comprise any initiation codon, the sequence homologous to an open reading frame is not itself an open reading frame since it does not serve as a template for translation.

In one embodiment, the 5′ replication recognition sequence comprises a sequence homologous to an open reading frame of a non-structural protein or a fragment thereof from an alphavirus, wherein the sequence homologous to an open reading frame of a non-structural protein or a fragment thereof from an alphavirus is characterized in that it comprises the removal of at least one initiation codon compared to the native alphavirus sequence.

In an embodiment, the sequence homologous to an open reading frame of a non-structural protein or a fragment thereof from an alphavirus is characterized in that it comprises the removal of at least the native start codon of the open reading frame of a non-structural protein. Preferably, it is characterized in that it comprises the removal of at least the native start codon of the open reading frame encoding nsP1.

The native start codon is the AUG base triplet at which translation on ribosomes in a host cell begins when an RNA is present in a host cell. In other words, the native start codon is the first base triplet that is translated during ribosomal protein synthesis, e.g., in a host cell that has been inoculated with RNA comprising the native start codon. In one embodiment, the host cell is a cell from a eukaryotic species that is a natural host of the specific alphavirus that comprises the native alphavirus 5′ replication recognition sequence. In an embodiment, the host cell is a BHK21 cell from the cell line “BHK21 [C13] (ATCC® CCL10™)”, available from American Type Culture Collection, Manassas, Virginia, USA.

The genomes of many alphaviruses have been fully sequenced and are publicly accessible, and the sequences of non-structural proteins encoded by these genomes are publicly accessible as well. Such sequence information allows to determine the native start codon in silico.

In an embodiment, the sequence homologous to an open reading frame of a non-structural protein or a fragment thereof from an alphavirus is characterized in that it comprises the removal of one or more initiation codons other than the native start codon of the open reading frame of a non-structural protein. In an embodiment, said nucleic acid sequence is additionally characterized by the removal of the native start codon. For example, in addition to the removal of the native start codon, any one or two or three or four or more than four (e.g., five) initiation codons may be removed.

If an rRNA according to the invention is characterized by the removal of the native start codon, and optionally by the removal of one or more initiation codons other than the native start codon, of the open reading frame of a non-structural protein, the sequence homologous to an open reading frame is not itself an open reading frame since it does not serve as a template for translation.

The one or more initiation codon other than the native start codon that is removed, preferably in addition to removal of the native start codon, is preferably selected from an AUG base triplet that has the potential to initiate translation. An AUG base triplet that has the potential to initiate translation may be referred to as “potential initiation codon”. Whether a given AUG base triplet has the potential to initiate translation can be determined in silico or in a cell-based in vitro assay.

In one embodiment, it is determined in silico whether a given AUG base triplet has the potential to initiate translation: in that embodiment, the nucleotide sequence is examined, and an AUG base triplet is determined to have the potential to initiate translation if it is part of an AUGG sequence, preferably part of a Kozak sequence.

In one embodiment, it is determined in a cell-based in vitro assay whether a given AUG base triplet has the potential to initiate translation: an rRNA characterized by the removal of the native start codon and comprising the given AUG base triplet downstream of the position of the removal of the native start codon is introduced into a host cell. In one embodiment, the host cell is a cell from a eukaryotic species that is a natural host of the specific alphavirus that comprises the native alphavirus 5′ replication recognition sequence. In a preferred embodiment, the host cell is a BHK21 cell from the cell line “BHK21 [C13] (ATCC® CCL10™)”, available from American Type Culture Collection, Manassas, Virginia, USA. It is preferable that no further AUG base triplet is present between the position of the removal of the native start codon and the given AUG base triplet. If, following transfer of the rRNA-characterized by the removal of the native start codon and comprising the given AUG base triplet-into the host cell, translation is initiated at the given AUG base triplet, the given AUG base triplet is determined to have the potential to initiate translation. Whether translation is initiated can be determined by any suitable method known in the art. For example, the rRNA may encode, downstream of the given AUG base triplet and in-frame with the given AUG base triplet, a tag that facilitates detection of the translation product (if any), e.g. a myc-tag or a HA-tag; whether or not an expression product having the encoded tag is present may be determined e.g. by Western Blot. In this embodiment, it is preferable that no further AUG base triplet is present between the given AUG base triplet and the nucleic acid sequence encoding the tag. The cell-based in vitro assay can be performed individually for more than one given AUG base triplet: in each case, it is preferable that no further AUG base triplet is present between the position of the removal of the native start codon and the given AUG base triplet. This can be achieved by removing all AUG base triplets (if any) between the position of the removal of the native start codon and the given AUG base triplet. Thereby, the given AUG base triplet is the first AUG base triplet downstream of the position of the removal of the native start codon.

Preferably, the 5′ replication recognition sequence of an rRNA according to the present invention is characterized by the removal of all potential initiation codons. Thus, according to the invention, the 5′ replication recognition sequence preferably does not comprise an open reading frame that can be translated to protein.

In an embodiment, the 5′ replication recognition sequence of an rRNA according to the invention is characterized by a secondary structure that is equivalent to the (predicted) secondary structure of the 5′ replication recognition sequence of viral genomic RNA. To this end, the rRNA may comprise one or more nucleotide changes compensating for nucleotide pairing disruptions within one or more stem loops introduced by the removal of at least one initiation codon.

In an embodiment, the 5′ replication recognition sequence of an rRNA according to the invention is characterized by a secondary structure that is equivalent to the secondary structure of the 5′ replication recognition sequence of alphaviral genomic RNA. In a preferred embodiment, the 5′ replication recognition sequence of an rRNA according to the invention is characterized by a predicted secondary structure that is equivalent to the predicted secondary structure of the 5′ replication recognition sequence of alphaviral genomic RNA. According to the present invention, the secondary structure of an RNA molecule is preferably predicted by the web server for RNA secondary structure prediction http://rna.urmc.rochester.edu/RNAstructureWeb/Servers/Predict1/Predict1.html.

By comparing the secondary structure or predicted secondary structure of a 5′ replication recognition sequence of an rRNA characterized by the removal of at least one initiation codon compared to the native alphavirus 5′ replication recognition sequence, the presence or absence of a nucleotide pairing disruption can be identified. For example, at least one base pair may be absent at a given position, compared to a native alphavirus 5′ replication recognition sequence, e.g. a base pair within a stem loop, in particular the stem of the stem loop.

In an embodiment, one or more stem loops of the 5′ replication recognition sequence are not deleted or disrupted. More preferably, stem loops 3 and 4 are not deleted or disrupted. Preferably, none of the stem loops of the 5′ replication recognition sequence is deleted or disrupted.

In one embodiment, the removal of at least one initiation codon does not disrupt the secondary structure of the 5′ replication recognition sequence. In an alternative embodiment, the removal of at least one initiation codon does disrupt the secondary structure of the 5′ replication recognition sequence. In this embodiment, the removal of at least one initiation codon may be causative for the absence of at least one base pair at a given position, e.g. a base pair within a stem loop, compared to a native 5′ replication recognition sequence. If a base pair is absent within a stem loop, compared to a native 5′ replication recognition sequence, the removal of at least one initiation codon is determined to introduce a nucleotide pairing disruption within the stem loop. A base pair within a stem loop is typically a base pair in the stem of the stem loop.

In an embodiment, an rRNA according to the invention comprises one or more nucleotide changes compensating for nucleotide pairing disruptions within one or more stem loops introduced by the removal of at least one initiation codon.

If the removal of at least one initiation codon introduces a nucleotide pairing disruption within a stem loop, compared to a native 5′ replication recognition sequence, one or more nucleotide changes may be introduced which are expected to compensate for the nucleotide pairing disruption, and the secondary structure or predicted secondary structure obtained thereby may be compared to a native 5′ replication recognition sequence.

Based on the common general knowledge and on the disclosure herein, certain nucleotide changes can be expected by the skilled person to compensate for nucleotide pairing disruptions. For example, if a base pair is disrupted at a given position of the secondary structure or predicted secondary structure of a given 5′ replication recognition sequence of an rRNA characterized by the removal of at least one initiation codon, compared to the native 5′ replication recognition sequence, a nucleotide change that restores a base pair at that position, preferably without re-introducing an initiation codon, is expected to compensate for the nucleotide pairing disruption.

In an embodiment, the 5′ replication recognition sequence of an rRNA according to the invention does not overlap with, or does not comprise, a translatable nucleic acid sequence, i.e. translatable into a peptide or protein, in particular an nsP, in particular nsP1, or a fragment of any thereof. For a nucleotide sequence to be “translatable”, it requires the presence of an initiation codon; the initiation codon encodes the most N-terminal amino acid residue of the peptide or protein. In one embodiment, the 5′ replication recognition sequence of an rRNA according to the invention does not overlap with, or does not comprise, a translatable nucleic acid sequence encoding an N-terminal fragment of nsP1.

In some scenarios, an rRNA comprises at least one subgenomic promoter. In a preferred embodiment, the subgenomic promoter of the rRNA does not overlap with, or does not comprise, a translatable nucleic acid sequence, i.e. translatable into a peptide or protein, in particular an nsP, in particular nsP4, or a fragment of any thereof. In one embodiment, the subgenomic promoter of an rRNA does not overlap with, or does not comprise, a translatable nucleic acid sequence that encodes a C-terminal fragment of nsP4. An rRNA having a subgenomic promoter that does not overlap with, or does not comprise, a translatable nucleic acid sequence, e.g. translatable into the C-terminal fragment of nsP4, may be generated by deleting part of the coding sequence for nsP4 (typically the part encoding the N-terminal part of nsP4), and/or by removing AUG base triplets in the part of the coding sequence for nsP4 that has not been deleted. If AUG base triplets in the coding sequence for nsP4 or a part thereof are removed, the AUG base triplets that are removed are preferably potential initiation codons. Alternatively, if the subgenomic promoter does not overlap with a nucleic acid sequence that encodes nsP4, the entire nucleic acid sequence encoding nsP4 may be deleted.

In an embodiment, an rRNA according to the invention does not comprise an open reading frame encoding solely the N-terminal fragment of nsP1, and optionally does not comprise an open reading frame encoding solely the C-terminal fragment of nsP4.

In some embodiments, an rRNA according to the present invention does not comprise stem loop 2 (SL2) of the 5′ terminus of the genome of an alphavirus. According to Frolov et al., supra, stem loop 2 is a conserved secondary structure found at the 5′ terminus of the genome of an alphavirus, upstream of CSE 2, but is dispensable for replication.

An rRNA according to the present invention is preferably a single stranded RNA molecule. An rRNA according to the present invention is typically a (+) stranded RNA molecule. In one embodiment, an rRNA of the present invention is an isolated nucleic acid molecule. An rRNA according to the present invention comprises at least one modified nucleotide, and preferably comprises one or more sequence changes, in particular those detected by the methods disclosed herein for identifying sequence changes that restore or improve the function of an rRNA comprising at least one modified nucleotide.

In an embodiment, an rRNA comprises a modified 5′ regulatory region of a self-replicating RNA virus of SEQ ID NO: 1, which is preferably a modified version of the 5′ regulatory region of VEEV Trinidad donkey strain (Accession No. L01442), and which modified regulatory region comprises a point mutation at one or more of positions 67, 244, 245, 246, 248 of the 5′ regulatory region (SEQ ID NO: 1). Preferably the 5′ regulatory region further comprises a point mutation at position 4 of the 5′ regulatory region (SEQ ID NO: 1). The point mutation is preferably G4A, A67C, G244A, C245A, G246A, or C248A.

### Safety Features of Embodiments of the Present Invention

The following features are preferred in the present invention, alone or in any suitable combination:

The replicons of the present invention are not particle-forming. This means that, following inoculation of a host cell by a replicon of the present invention, the host cell does not produce virus particles, such as next generation virus particles. In one embodiment, an RNA replicon according to the invention is completely free of genetic information encoding any virus structural protein, e.g., alphavirus structural protein, such as core nucleocapsid protein C, envelope protein P62, and/or envelope protein E1. Preferably, the replicon according to the present invention does not comprise a virus packaging signal, e.g., an alphavirus packaging signal. For example, the alphavirus packaging signal comprised in the coding region of nsP2 of SFV (White et al., 1998, J. Virol. 72:4320-4326) may be removed, e.g. by deletion or mutation. A suitable way of removing the alphavirus packaging signal includes adaptation of the codon usage of the coding region of nsP2. The degeneration of the genetic code may allow to delete the function of the packaging signal without affecting the amino acid sequence of the encoded nsP2.

miRNA

The second RNA molecule of the present invention comprises, optionally encodes, at least one miRNA sequence, which miRNA sequence is capable of being excised from the second replicable RNA molecule when present in a cell, and is capable of regulating gene expression in a cell. The second RNA molecule of the present invention comprises, optionally encodes, at least one non-coding RNA sequence, which non-coding RNA sequence is capable of being excised from the second replicable RNA molecule when present in a cell, and is capable of regulating gene expression in a cell. Preferably the cell is a eukaryotic cell, preferably a mammalian, preferably a human cell. The cell in which the second RNA is to be present for excision typically has to be capable of excising the miRNA sequence from the second RNA molecule, for example it has to have the required enzymes such as Drosha and Dicer. The cell may endogenously (i.e., naturally) express the required factors (typically enzymes), or alternatively may have been modified to express the required factors (typically enzymes), needed for excising the non-coding RNA sequence, preferably the miRNA sequence, from the second RNA molecule. Such factors, typically enzymes, may be capable of excising a sequence containing the miRNA sequence from the second RNA molecule and may further processes the sequence as required to provide a functional miRNA sequence.

The miRNA capable of being excised from the second RNA molecule inside a cell is typically flanked by flanking sequences up- and/or downstream of the miRNA. These flanking sequences serve as or comprise recognition sequences for excision of the miRNA from the second RNA molecule. Thus, the factors or enzymes as described above may target the recognition sequences in the flanking sequences to effect excision of the miRNA from the second RNA molecule.

In an embodiment, the flanking sequences up- and/or downstream of the at least one miRNA sequence are flanking sequences that are naturally occurring flanking sequences, for example, sequences that flank naturally occurring miRNAs, such as from murine miR-155. In case the miRNA is a naturally occurring miRNA, the flanking sequences can be flanking sequences that also flank the miRNA sequence in nature or they can be flanking sequences that do not flank the miRNA in nature, such as flanking sequences that flank other miRNA sequences. The flanking sequences can be from the same or from different organisms as the miRNA sequence.

In an embodiment, the flanking sequences up- and/or downstream of the at least one miRNA sequence are artificial flanking sequences.

The term “capable of regulating gene expression” means that the miRNA is influencing the expression level of a certain gene product, such as a gene-encoded a protein, whereby the level of the protein is regulated. The regulation can be a complete stop of the expression, also known as silencing, of a gene or the attenuation of expression, which means that less of the gene is expressed, or enhancing expression. Preferably regulation is done by targeting an mRNA to prevent its translation.

The target of the miRNA is not particularly limited. Preferably the target is of particular interest for the onset or progression of a disease or disorder and its regulation helps in treating or preventing this disease or disorder. The target can also be relevant for inducing pluripotency.

The term “targeting” means according to the invention binding of the miRNA to an at least partially complementary sequence, preferably of an mRNA, and regulating the expression from the mRNA.

The origin of the miRNA sequence can be natural or artificial. A natural miRNA sequence originates preferably from the same organism in which the RNA molecules of the present invention are to be introduced. For example, when it is foreseen to introduce the system of the present invention into a human cell, the miRNA is preferably of human origin.

An artificial pre-miRNA sequence can also comprise a naturally occurring mature miRNA sequence. In this embodiment, for example, the sequence of a naturally occurring mature miRNA is included in an artificial pre-miRNA where the flanking and loop sequences are not those naturally associated with this mature miRNA.

An miRNA sequence also may be designed to be at least partially complementary to, for example capable of binding to, a particular mRNA of interest, i.e., a target mRNA. Thus, the second RNA molecule may comprise a miRNA sequence which is at least partially complementary to (i.e., targets) an mRNA of interest, optionally further comprising flanking sequences as described herein.

The term “mature miRNA” or “functional miRNA” are used interchangeably in this application. They refer to an miRNA of about 22 nucleotides which is capable of directly regulating gene expression by binding together with proteins to its target, e.g., target mRNA.

In some embodiments, the miRNA sequence comprised on the second RNA molecule may be 10-200 nucleotides in length, optionally 10-100, 10-90, 10-80, 10-70, 10-60, 10-50, 10-40, 10-30, 20-100, 20-90, 20-80, 20-70, 20-60, 20-50, 20-40, or 20-30 nucleotides in length, optionally 10-50, preferably 10-30 nucleotides in length.

### At Least One Open Reading Frame Encoding at Least One Gene Product of Interest

In one embodiment, the first and/or second RNA according to the present invention, preferably the second RNA molecule, comprises at least one open reading frame encoding a gene product of interest, such as a protein of interest. Preferably, the protein of interest is encoded by a heterologous nucleic acid sequence. The gene encoding the protein of interest is synonymously termed “gene of interest” or “transgene”. In various embodiments, the protein of interest is encoded by a heterologous nucleic acid sequence. According to the present invention, the term “heterologous” refers preferably to the fact that a nucleic acid sequence is not naturally functionally or structurally linked to a virus nucleic acid sequence, e.g., an alphavirus nucleic acid sequence.

In some embodiments, the first and/or second RNA according to the present invention may comprise more than one open reading frames encoding a protein of interest, each of which may independently be selected to be under the control of a subgenomic promoter or not. Alternatively, a poly-protein or fusion polypeptide comprises individual polypeptides separated by a 2A self-cleaving peptides (e.g. from foot-and-mouth disease virus 2A protein), or protease cleavage site or an intein.

### Position of the at Least One Open Reading Frame Encoding a Protein of Interest

The first and second RNA are suitable for expression of one or more genes encoding a protein of interest, optionally under control of a subgenomic promoter. Various embodiments are possible. One or more open reading frames, each encoding a protein of interest, can be present on the first and/or second RNA, preferably the second RNA. The most upstream open reading frame of each RNA is referred to as “first open reading frame”. In one embodiment, on the first RNA the one or more open reading frame encoding a protein of interest is located downstream of the open reading frame encoding a functional non-structural protein. In one embodiment, the first open reading frame encoding a protein of interest is located downstream from the 5′ replication recognition sequence and, in case of the first RNA, optionally the open reading frame encoding one or more non-structural proteins from a self-replicating virus. In one embodiment, the first open reading frame encoding a protein of interest is located downstream from the 5′ replication recognition sequence and, in case of the first RNA, upstream from an IRES and optionally the open reading frame encoding one or more non-structural proteins from a self-replicating virus. In some embodiments, one or more further open reading frames can be present downstream of the first open reading frame. One or more further open reading frames downstream of the first open reading frame may be referred to as “second open reading frame”, “third open reading frame” and so on, in the order (5′ to 3′) in which they are present downstream of the first open reading frame. In one embodiment, on the first RNA one or more further open reading frames encoding one or more proteins of interest are located downstream from the open reading frame encoding one or more non-structural proteins from a self-replicating virus and are preferably controlled by subgenomic promotors. Preferably each open reading frame encoding a protein of interest is controlled by a subgenomic promoter. Preferably, each open reading frame comprises a start codon (base triplet), typically AUG (in the RNA molecule), corresponding to ATG (in a respective DNA molecule).

If a replicon comprises a 3′ replication recognition sequence, it is preferred that all open reading frames are localized upstream of the 3′ replication recognition sequence.

In some embodiments, at least one open reading frame of the first and/or second RNA is under the control of a subgenomic promoter, preferably an alphavirus subgenomic promoter. The alphavirus subgenomic promoter is very efficient, and is therefore suitable for heterologous gene expression at high levels. Preferably, the subgenomic promoter is a promoter for a subgenomic transcript in an alphavirus. This means that the subgenomic promoter is one which is native to an alphavirus and which preferably controls transcription of the open reading frame encoding one or more structural proteins in said alphavirus. Alternatively, the subgenomic promoter is a variant of a subgenomic promoter of an alphavirus; any variant which functions as promoter for subgenomic RNA transcription in a host cell is suitable. If the first and/or second RNA comprises a subgenomic promoter, it is preferred that the first and/or second RNA comprises a conserved sequence element 3 (CSE 3) or a variant thereof.

Preferably, the at least one open reading frame under control of a subgenomic promoter is localized downstream of the subgenomic promoter. Preferably, the subgenomic promoter controls production of subgenomic RNA comprising a transcript of the open reading frame.

In some embodiments the first open reading frame is under control of a subgenomic promoter. In one embodiment, when the first open reading frame is under control of the subgenomic promoter, the gene encoded by the first open reading frame can be expressed both from the RNA as well as from a subgenomic transcript thereof (the latter in the presence of functional alphavirus replicase). One or more further open reading frames, each under control of a subgenomic promoter, may be present downstream of the first open reading frame that may be under control of a subgenomic promoter. The proteins encoded by the one or more further open reading frames, e.g. by the second open reading frame, may be translated from one or more subgenomic transcripts, each under control of a subgenomic promoter. For example, the first RNA may comprise a subgenomic promoter controlling production of a transcript that encodes a third protein of interest.

In other embodiments the first open reading frame is not under control of a subgenomic promoter. In one embodiment, when the first open reading frame is not under control of a subgenomic promoter, the protein encoded by the first open reading frame can be expressed from the RNA. One or more further open reading frames, each under control of a subgenomic promoter, may be present downstream of the first open reading frame. The proteins encoded by the one or more further open reading frames may be expressed from subgenomic transcripts.

In a cell which comprises the first and second RNA according to the present invention, the second and optionally first RNA may be amplified by functional replicase. Additionally, if the first and/or second RNA comprises one or more open reading frames under control of a subgenomic promoter, one or more subgenomic transcripts are expected to be produced by functional replicase.

If a first and/or second RNA comprises more than one open reading frame encoding a protein of interest, it is preferable that each open reading frame encodes a different protein. For example, the protein encoded by the second open reading frame encoding a protein of interest is different from the protein encoded by the first open reading frame encoding a protein of interest.

### IRES

In an embodiment, a first RNA may comprise an internal ribosome entry site (IRES) and an open reading frame encoding one or more non-structural proteins from a self-replicating virus, wherein the IRES controls expression of the one or more non-structural proteins, e.g., nsp1234. Preferably, the first and/or second rRNA contains sequence elements allowing replication by a functional replicase. In one embodiment, the self-replicating virus is an alphavirus and the sequence elements allowing replication by the functional replicase are derived from an alphavirus.

Alphavirus replicases have a capping enzyme function, and, typically, genomic as well as subgenomic (+) stranded RNAs are capped. The 5′-cap serves to protect mRNA from degradation, and to direct the ribosomal subunits as well as cellular factors to the mRNA in order to form a ribonucleoprotein complex on the mRNA that then can start translation from a nearby start codon. This complex process is extensively described in the literature (Jackson et al., 2010, Nat Rev Mol Biol; Vol 10:113-127). Despite the very elaborated and efficient mechanism of cap dependent translation, cells have means to initiate translation fully or partially independently from the 5′ cap (Thompson 2012; Trends in Microbiology 20:558-566). Thereby, in situations of cellular stress that lead to a global down regulation of cap-dependent translation, the cells may still express selected genes preferentially, often with the help of an IRES.

Viruses also evolved different means to exploit the cells machinery for translation of the viral genes. Since a viral infection is often sensed by the cell which leads to cellular antiviral response (interferon response; stress response), many viruses also make use of cap-independent translation, especially RNA viruses. Cap independent translation ensure an advantage for the viral RNA translation upon cellular stress response giving the viruses the opportunity to fulfil their life cycle and be released from infected cells.

Internal ribosomal entry sites (IRESs) are RNA sequences forming appropriate secondary structures that attract the pre-initiation complex near to a translational start codon, AUG or others. Four classes of IRESs are described in literature that share common features. Prototypic IRESs are the poliovirus IRES (Type I), the encephalomyocarditis virus (EMCV) IRES (Type II), the hepatitis C virus (HCV) IRES (Type III) and the IRES found in the intergenic regions of dicistroviruses (Type IV) (Thompson, 2012; Trends in Microbiology 20:558-566; Lozano et al., 2018; Open Biology 8:180155).

Type I to III IRESs have in common that they initiate translation at AUG start codons, whereas type IV IRES initiate at non-AUG codons (e.g., GCU). Thereby Type I to III require the initiator tRNA that delivers methionine by the help of eIF2/GTP (eIF2/GTP/Met-tRNAiMet). Activation of eIF2 kinases under stress phosphorylates the alpha subunit of eIF2 which inhibits translation that initiates at AUG. Thereby translation directed by type IV IRESs are not inhibited by eIF2 phosphorylation.

The term “internal ribosome entry site”, abbreviated “IRES”, relates to an RNA element that recruits ribosomes to the internal region of mRNAs to initiate translation in a cap-independent manner. IRESs are commonly located in the 5′-UTR of RNA viruses. However, mRNAs of viruses from dicistroviridae family possess two open reading frames (ORFs), and translation of each is directed by two distinct IRESs. It has also been suggested that some mammalian cellular mRNAs also have IRESs. These cellular IRES elements are thought to be located in eukaryotic mRNAs encoding genes involved in stress survival, and other processes critical to survival. The location for IRES elements is often in the 5′-UTR, but can also occur elsewhere in mRNAs.

The term “internal ribosome entry site” includes IRESs that are present in the viruses of the Picornaviridae family such as poliovirus (PV) and encephalomyocarditis virus and pathogenic viruses, including human immunodeficiency virus, hepatitis C virus (HCV), and foot and mouth disease virus. Although these viral IRESs contain diverse sequences, many of them have similar secondary structures and initiate translation through similar mechanisms. In addition, the activities of IRESs often require assistance from other factors known as IRES-transacting factors (ITAFs). Based on the structures and the requirement of translation initiation factors (IFs) and ITAFs, the viral IRESs are classified into four types as described herein. Any of these IRES types is useful according to the invention, with Type IV IRESs being particularly preferred.

Two groups of viral IRESs, Type I and Type II, cannot bind to the 40S small ribosomal subunit directly. Instead, they recruit the 40S small ribosomal subunit through different ITAFs and require canonical IFs in the cap-dependent translation (i.e., eIF2, eIF3, eIF4A, eIF4B, and eIF4G). The major difference between Type I and Type II IRESs is the requirement of 40S ribosome scanning, with 40S ribosome scanning being unnecessary for Type II IRES. Examples of Type I IRESs include IRESs found in poliovirus (PV) and rhinovirus. Examples of Type II IRESs include IRESs found in encephalomyocarditis virus (EMCV), foot-and-mouth disease virus (FMDV) and Theiler's murine encephalomyelitis viruses (TMEV).

Type III IRESs can directly interact with 40S small ribosomal subunit with specialized RNA structure, but their activities usually require assistance of several IFs including eIF2 and eIF3 and initiator Met-tRNAi. Examples include IRESs found in hepatitis C-virus (HCV), classical swine fever virus (CSFV) and porcine teschovirus (PTV).

Type IV viral IRESs generally have strong activities and can initiate translation from a non-AUG start codon without additional ITAFs or even eIF2/Met-tRNAi/GTP ternary complex. These IRESs are folded to a compact structure that directly interacts with the 40S small ribosomal subunit. Examples include IRESs found in dicistroviruses such as cricket paralysis virus (CrPV), Plautia stali intestine virus (PSIV), and Taura-Syndrom-Virus (TSV).

The term “internal ribosome entry site” also includes IRESs found in cellular mRNAs, many of which encode proteins required in stress response, e.g. in conditions of apoptosis, mitosis, hypoxia, and nutrient limitation. The cellular IRESs can be roughly classified into two types based on the mechanisms of ribosome recruitment: Type I IRESs interact with ribosomes through ITAFs that bound on the cis-elements, e.g., RNA binding motifs and N-6-methyladenosine (m6A) modification, whereas Type II IRESs contain a short cis-element that pairs with 18S rRNA to recruit ribosomes.

### Protein of Interest

Proteins of interest may, e.g., be selected from the group consisting of reporter proteins, pharmaceutically active peptides or proteins, inhibitors of intracellular interferon (IFN) signaling, pluripotency factors, differentiation factors, vaccinia virus immune evasion proteins or antigens or epitopes thereof. According to the invention, a protein of interest preferably does not include functional non-structural proteins from a self-replicating virus, e.g., functional alphavirus non-structural proteins.

### Reporter Protein

In one embodiment, an open reading frame encodes a reporter protein, e.g., a cell-surface expressed protein such as CD90. In that embodiment, the open reading frame comprises a reporter gene. Certain genes may be chosen as reporters because the characteristics they confer on cells or organisms expressing them may be readily identified and measured, or because they are selectable markers. Reporter genes are often used as an indication of whether a certain gene has been taken up by or expressed in the cell or organism population. Preferably, the expression product of the reporter gene is visually detectable. Common visually detectable reporter proteins typically possess fluorescent or luminescent proteins. Examples of specific reporter genes include the gene that encodes jellyfish green fluorescent protein (GFP), which causes cells that express it to glow green under blue light, the enzyme luciferase (Luc), which catalyzes a reaction with luciferin to produce light, and the red fluorescent protein (RFP). Variants of any of these specific reporter genes are possible, as long as the variants possess visually detectable properties. For example, eGFP is a point mutant variant of GFP. The reporter protein embodiment is particularly suitable for testing expression.

### Pharmaceutically Active Peptide or Protein

According to the invention, in one embodiment, the first and/or second RNA comprises or consists of pharmaceutically active RNA. A “pharmaceutically active RNA” may be RNA that encodes a pharmaceutically active peptide or protein. Preferably, the RNA according to the present invention encodes a pharmaceutically active peptide or protein. Preferably, the RNA according to the present invention comprises a pharmaceutically active miRNA. In some embodiments, the system according to the present invention encodes a pharmaceutically active peptide or protein, and a pharmaceutically active miRNA. Preferably the first RNA molecule encodes a replicase as described herein, and the second replicable RNA molecule, which is capable of being replicated in trans by the replicase encoded by the first RNA molecule, encodes a pharmaceutically active peptide or protein, and a pharmaceutically active miRNA. Preferably, an open reading frame encodes a pharmaceutically active peptide or protein. Preferably, the RNA comprises an open reading frame that encodes a pharmaceutically active peptide or protein, optionally under control of the subgenomic promoter.

A “pharmaceutically active peptide or protein” or a “pharmaceutically active miRNA” has a positive or advantageous effect on the condition or disease state of a subject when administered to the subject in a therapeutically effective amount. Preferably, a pharmaceutically active peptide or protein or a pharmaceutically active miRNA has curative or palliative properties and may be administered to ameliorate, relieve, alleviate, reverse, delay onset of or lessen the severity of one or more symptoms of a disease or disorder. A pharmaceutically active peptide or protein or a pharmaceutically active miRNA may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., the peptide or protein elicits an immune response in a subject which may be therapeutic or partially or fully protective.

In one embodiment, the pharmaceutically active peptide or protein is or comprises an immunologically active compound or an antigen or an epitope.

According to the invention, the term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. In one embodiment, the immune response involves stimulation of an antibody response (usually including immunoglobulin G (IgG)). Immunologically active compounds possess potent immunostimulating activity including, but not limited to, antiviral and antitumor activity, and can also down-regulate other aspects of the immune response, for example shifting the immune response away from a Th2 immune response, which is useful for treating a wide range of Th2 mediated diseases.

According to the invention, the term “antigen” or “immunogen” covers any substance that will elicit an immune response. In particular, an “antigen” relates to any substance that reacts specifically with antibodies or T-lymphocytes (T-cells). According to the present invention, the term “antigen” comprises any molecule which comprises at least one epitope. Preferably, an antigen in the context of the present invention is a molecule which, optionally after processing, induces an immune reaction, which is preferably specific for the antigen. According to the present invention, any suitable antigen may be used, which is a candidate for an immune reaction, wherein the immune reaction may be both a humoral as well as a cellular immune reaction. In the context of the embodiments of the present invention, the antigen is preferably presented by a cell, preferably by an antigen presenting cell, in the context of MHC molecules, which results in an immune reaction against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present invention, an antigen may correspond to a naturally occurring product, for example, a viral protein, or a part thereof. In preferred embodiments, the antigen is a surface polypeptide, i.e. a polypeptide naturally displayed on the surface of a cell, a pathogen, a bacterium, a virus, a fungus, a parasite, an allergen, or a tumor. The antigen may elicit an immune response against a cell, a pathogen, a bacterium, a virus, a fungus, a parasite, an allergen, or a tumor.

The term “pathogen” refers to pathogenic biological material capable of causing disease in an organism, preferably a vertebrate organism. Pathogens include microorganisms such as bacteria, unicellular eukaryotic organisms (protozoa), fungi, parasites as well as viruses.

The terms “epitope”, “antigen peptide”, “antigen epitope”, “immunogenic peptide” and “MHC binding peptide” are used interchangeably herein and refer to an antigenic determinant in a molecule such as an antigen, i.e., to a part in or fragment of an immunologically active compound that is recognized by the immune system, for example, that is recognized by a T cell, in particular when presented in the context of MHC molecules. An epitope of a protein preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 9, 10, 11, 12, 13, 14, 15, 16, 17, 18, 19, 20, 21, 22, 23, 24, or 25 amino acids in length. According to the invention an epitope may bind to MHC molecules such as MHC molecules on the surface of a cell and thus, may be a “MHC binding peptide” or “antigen peptide”. The term “major histocompatibility complex” and the abbreviation “MHC” include MHC class I and MHC class II molecules and relate to a complex of genes which is present in all vertebrates. MHC proteins or molecules are important for signaling between lymphocytes and antigen presenting cells or diseased cells in immune reactions, wherein the MHC proteins or molecules bind peptides and present them for recognition by T cell receptors. The proteins encoded by the MHC are expressed on the surface of cells, and display both self-antigens (peptide fragments from the cell itself) and non-self-antigens (e.g., fragments of invading microorganisms) to a T cell. Preferred such immunogenic portions bind to an MHC class I or class II molecule. As used herein, an immunogenic portion is said to “bind to” an MHC class I or class II molecule if such binding is detectable using any assay known in the art. The term “MHC binding peptide” relates to a peptide which binds to an MHC class I and/or an MHC class II molecule. In the case of class I MHC/peptide complexes, the binding peptides are typically 8-10 amino acids long although longer or shorter peptides may be effective. In the case of class II MHC/peptide complexes, the binding peptides are typically 10-25 amino acids long and are in particular 13-18 amino acids long, whereas longer and shorter peptides may be effective.

In an embodiment, the protein of interest according to the present invention comprises an epitope suitable for vaccination of a target organism. A person skilled in the art will know that one of the principles of immunobiology and vaccination is based on the fact that an immunoprotective reaction to a disease is produced by immunizing an organism with an antigen, which is immunologically relevant with respect to the disease to be treated. An antigen is selected from the group comprising a self-antigen and non-self-antigen. A non-self-antigen is preferably a bacterial antigen, a virus antigen, a fungus antigen, an allergen or a parasite antigen. It is preferred that the antigen comprises an epitope that is capable of eliciting an immune response in a target organism. For example, the epitope may elicit an immune response against a bacterium, a virus, a fungus, a parasite, an allergen, or a tumor, such as a cytotoxic T cell response.

In some embodiments the non-self-antigen is a bacterial antigen. In some embodiments, the antigen elicits an immune response against a bacterium which infects animals, including birds, fish and mammals, including domesticated animals. Preferably, the bacterium against which the immune response is elicited is a pathogenic bacterium.

In some embodiments the non-self-antigen is a virus antigen. A virus antigen may for example be a peptide from a virus surface protein, e.g. a capsid polypeptide or a spike polypeptide, such as from Coronavirus. In some embodiments, the antigen elicits an immune response against a virus which infects animals, including birds, fish and mammals, including domesticated animals. Preferably, the virus against which the immune response is elicited is a pathogenic virus, such as Ebola virus.

In some embodiments the non-self-antigen is a polypeptide or a protein from a fungus. In some embodiments, the antigen elicits an immune response against a fungus which infects animals, including birds, fish and mammals, including domesticated animals. Preferably, the fungus against which the immune response is elicited is a pathogenic fungus.

In some embodiments the non-self-antigen is a polypeptide or protein from a unicellular eukaryotic parasite. In some embodiments, the antigen elicits an immune response against a unicellular eukaryotic parasite, preferably a pathogenic unicellular eukaryotic parasite. Pathogenic unicellular eukaryotic parasites may be e.g. from the genus Plasmodium, e.g. P. falciparum, P. vivax, P. malariae or P. ovale, from the genus Leishmania, or from the genus Trypanosoma, e.g. T. cruzi or T. brucei.

In some embodiments, it is not required that the pharmaceutically active peptide or protein is an antigen that elicits an immune response. Suitable pharmaceutically active proteins or peptides may be selected from the group consisting of cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, chimeric antigen receptors (CARs), immunoglobulins), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Willebrand factor, antithrombin III, glucocerebrosidase, erythropoietin granulocyte colony stimulating factor (GCSF) or modified Factor VIII, anticoagulants and the like. In one embodiment, the pharmaceutically active protein according to the invention is a cytokine which is involved in regulating lymphoid homeostasis, preferably a cytokine which is involved in and preferably induces or enhances development, priming, expansion, differentiation and/or survival of T cells. In one embodiment, the cytokine is an interleukin, e.g. IL-2, IL-7, IL-12, IL-15, or IL-21.

A further suitable protein of interest encoded by an open reading frame is an inhibitor of interferon (IFN) signaling. While it has been reported that viability of cells in which RNA has been introduced for expression can be reduced, in particular, if cells are transfected multiple times with RNA, IFN inhibiting agents were found to enhance the viability of cells in which RNA is to be expressed (WO 2014/071963 A1). Preferably, the inhibitor is an inhibitor of IFN type I signaling. Preventing engagement of IFN receptor by extracellular IFN and inhibiting intracellular IFN signaling in the cells allows stable expression of RNA in the cells. Alternatively or additionally, preventing engagement of IFN receptor by extracellular IFN and inhibiting intracellular IFN signaling enhances survival of the cells, in particular, if cells are transfected repetitively with RNA. Without wishing to be bound by theory, it is envisaged that intracellular IFN signaling can result in inhibition of translation and/or RNA degradation. This can be addressed by inhibiting one or more IFN-inducible antivirally active effector proteins. The IFN-inducible antivirally active effector protein can be selected from the group consisting of RNA-dependent protein kinase (PKR), 2′,5′-oligoadenylate synthetase (OAS) and RNaseL. Inhibiting intracellular IFN signaling may comprise inhibiting the PKR-dependent pathway and/or the OAS-dependent pathway. A suitable protein of interest is a protein that is capable of inhibiting the PKR-dependent pathway and/or the OAS-dependent pathway. Inhibiting the PKR-dependent pathway may comprise inhibiting elF2-alpha phosphorylation. Inhibiting PKR may comprise treating the cell with at least one PKR inhibitor. The PKR inhibitor may be a viral inhibitor of PKR. The preferred viral inhibitor of PKR is vaccinia virus E3. If a peptide or protein (e.g. E3, K3) is to inhibit intracellular IFN signaling, intracellular expression of the peptide or protein is preferred. Vaccinia virus E3 is a 25 kDa dsRNA-binding protein (encoded by gene E3L) that binds and sequesters dsRNA to prevent the activation of PKR and OAS. E3 can bind directly to PKR and inhibits its activity, resulting in reduced phosphorylation of elF2-alpha. A further preferred viral inhibitor is Vaccinia virus B18, in particular B18R. Vaccinia virus B18 is a soluble inhibitor of IFN-alpha with a molecular weight of 41 kDa. Other suitable inhibitors of IFN signaling are Herpes simplex virus ICP34.5, Toscana virus NSs, Bombyx mori nucleopolyhedrovirus PK2, and HCV NS34A.

### Pluripotency Factor

The term “pluripotency factors” or “reprogramming transcription factors” relates to molecules, in particular peptides or proteins, which, when expressed in somatic cells optionally together with further agents such as further reprogramming factors, lead to reprogramming or de-differentiation of said somatic cells to cells having stem cell characteristics, in particular pluripotency. Particular examples of reprogramming factors include OCT4, SOX2, c-MYC, KLF4, LIN28, and NANOG.

### Differentiation Factor

The protein of interest encoded by an RNA molecule can preferably be a differentiation factor. This factor can be used for (trans) differentiation, which means that upon introduction of such a factor into a, preferably already differentiated, cell, the cell is (re) programmed into a (different) specific cell type. Transdifferentiation means in particular that a state of pluripotency is not occurring for reprogramming of a cell from one cell type to another. Examples of such a protein of interest is MYOD1, which can also be used as a transdifferentiation factor for reprogramming a fibroblast into a muscle cell.

### Methods of Preparing RNA

The RNA molecules according to the present invention may be obtainable by in vitro transcription. In vitro-transcribed RNA (IVT-RNA) is of particular interest in the present invention. IVT-RNA is obtainable by transcription from a nucleic acid molecule (particularly a DNA molecule). The DNA molecule(s) of the present invention are suitable for such purposes, particularly if comprising a promoter that can be recognized by a DNA-dependent RNA-polymerase.

RNA according to the present invention can be synthesized in vitro. This allows to add cap-analogs to the in vitro transcription reaction. Typically, the poly(A) tail is encoded by a poly-(dT) sequence on the DNA template. Alternatively, capping and poly(A) tail addition can be achieved enzymatically after transcription.

The in vitro transcription methodology is known to the skilled person. For example, as mentioned in WO 2011/015347 A1, a variety of in vitro transcription kits is commercially available.

### DNA

The present invention also provides a DNA comprising a nucleic acid sequence encoding an RNA according to the present invention.

Preferably, the DNA is double-stranded.

In a preferred embodiment, the DNA is a plasmid. The term “plasmid”, as used herein, generally relates to a construct of extrachromosomal genetic material, usually a circular DNA duplex, which can replicate independently of chromosomal DNA.

The DNA of the present invention may comprise a promoter that can be recognized by a DNA-dependent RNA-polymerase. This allows for transcription of the encoded RNA in vivo or in vitro, e.g. of the RNA of the present invention. IVT vectors may be used in a standardized manner as template for in vitro transcription. Examples of promoters preferred according to the invention are promoters for SP6, T3 or T7 polymerase.

In one embodiment, the DNA of the present invention is an isolated nucleic acid molecule.

### Further Components of the System

The system described herein may be present in the form of a composition or two separate compositions. The system may comprise further components. The following embodiments relating to a system apply to embodiments wherein the system is a composition or separate compositions wherein, for example, only one of the RNAs is present.

In one embodiment of the present invention, a system can further comprise a solvent such as an aqueous solvent or any solvent that makes it possible to preserve the integrity of the RNA. In a preferred embodiment, the system is an aqueous solution comprising RNA. The aqueous solution may optionally comprise solutes, e.g. salts.

In one embodiment of the present invention, the system is in the form of a freeze-dried composition or at least two freeze-dried compositions. A freeze-dried composition is obtainable by freeze-drying a respective aqueous composition.

In some embodiments, the systems as described herein may further comprise a reagent capable of forming particles with the RNA molecules.

A system described herein may additionally comprise salts, buffers, or other components as further described below.

In some embodiments, a salt for use in the systems described herein comprises sodium chloride. Without wishing to be bound by theory, sodium chloride functions as an ionic osmolality agent for preconditioning RNA prior to mixing with lipids. In some embodiments, the systems described herein may comprise alternative organic or inorganic salts. Alternative salts include, without limitation, potassium chloride, dipotassium phosphate, monopotassium phosphate, potassium acetate, potassium bicarbonate, potassium sulfate, disodium phosphate, monosodium phosphate, sodium acetate, sodium bicarbonate, sodium sulfate, lithium chloride, magnesium chloride, magnesium phosphate, calcium chloride, and sodium salts of ethylenediaminetetraacetic acid (EDTA).

Generally, systems or compositions for storing RNA particles such as for freezing RNA particles comprise low sodium chloride concentrations, or comprises a low ionic strength. In some embodiments, the sodium chloride is at a concentration from 0 mM to about 50 mM, from 0 mM to about 40 mM, or from about 10 mM to about 50 mM.

According to the present disclosure, the systems described herein have a pH suitable for the stability of the RNA particles and, in particular, for the stability of the RNA. Without wishing to be bound by theory, the use of a buffer system maintains the pH of the particle compositions described herein during manufacturing, storage and use of the compositions. In some embodiments of the present disclosure, the buffer system may comprise a solvent (in particular, water, such as deionized water, in particular water for injection) and a buffering substance. The buffering substance may be selected from 2-[4-(2-hydroxyethyl) piperazin-1-yl]ethanesulfonic acid (HEPES), 2-amino-2-(hydroxymethyl) propane-1,3-diol (Tris), acetate, and histidine. A preferred buffering substance is HEPES.

Systems described herein may also comprise a cryoprotectant and/or a surfactant as stabilizer to avoid substantial loss of the product quality and, in particular, substantial loss of RNA activity during storage, freezing, spray-drying and/or lyophilization, for example to reduce or prevent aggregation, particle collapse, RNA degradation and/or other types of damage.

In an embodiment, the cryoprotectant is a carbohydrate. The term “carbohydrate”, as used herein, refers to and encompasses monosaccharides, disaccharides, trisaccharides, oligosaccharides and polysaccharides.

In an embodiment, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In an embodiment, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like.

The term “trisaccharide” means three sugars linked together to form one molecule. Examples of a trisaccharides include raffinose and melezitose.

In an embodiment, the cryoprotectant is an oligosaccharide. The term “oligosaccharide”, as used herein refers to a compound or a chemical moiety formed by 3 to about 15, such as 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In an embodiment, the cryoprotectant is a cyclic oligosaccharide. The term “cyclic oligosaccharide”, as used herein refers to a compound or a chemical moiety formed by 3 to about 15, such as 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as a cyclodextrin, β cyclodextrin, or γcyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an a, B, or γcyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, systems may include sucrose. Without wishing to be bound by theory, sucrose functions to promote cryoprotection, thereby preventing RNA (especially rRNA) particle aggregation and maintaining chemical and physical stability of the composition. In some embodiments, systems may include alternative cryoprotectants to sucrose. Alternative stabilizers include, without limitation, trehalose and glucose. In a specific embodiment, an alternative stabilizer to sucrose is trehalose or a mixture of sucrose and trehalose.

A preferred cryoprotectant is selected from the group consisting of sucrose, trehalose, glucose, and a combination thereof, such as a combination of sucrose and trehalose. In a preferred embodiment, the cryoprotectant is sucrose. Some embodiments of the present disclosure contemplate the use of a chelating agent in a system described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In some embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In an alternative embodiment, the systems described herein do not comprise a chelating agent.

Terms such as “stability” or “desired storage stability” as used herein may refer to physicochemical stability of the product, e.g., Tris/sucrose finished product, in unopened thawed vials for up to 24 hours at 30 °C, and in syringes for up to 24 hours at 2-8 °C and 12 hours at 30 °C. Such terms may refer to shelf-life for the product of 6 months or more when stored at-90 to-60 °C.

In some embodiments, the system of the invention may comprise one or more adjuvants. Adjuvants may be added to vaccines to stimulate the immune system's response; adjuvants do not typically provide immunity themselves. Exemplary adjuvants include without limitation the following: Inorganic compounds (e.g. alum, aluminum hydroxide, aluminum phosphate, calcium phosphate hydroxide); mineral oil (e.g. paraffin oil), cytokines (e.g. IL-1, IL-2, IL-12); immunostimulatory polynucleotide (such as RNA or DNA; e.g., CpG-containing oligonucleotides); saponins (e.g. plant saponins from Quillaja, Soybean, Polygala senega); oil emulsions or liposomes; polyoxy ethylene ether and poly oxy ethylene ester formulations; polyphosphazene (PCPP); muramyl peptides; imidazoquinolone compounds; thiosemicarbazone compounds; the Flt3 ligand (WO 2010/066418 A1); or any other adjuvant that is known by a person skilled in the art. A preferred adjuvant for administration of RNA according to the present invention is the Flt3 ligand (WO 2010/066418 A1). When Flt3 ligand is administered together with RNA that codes for an antigen, a strong increase in antigen-specific CD8+ T cells may be observed.

The system according to the invention can be buffered, (e.g., with an acetate buffer, a citrate buffer, a succinate buffer, a Tris buffer, a phosphate buffer).

### RNA-Containing Particles

In some embodiments, owing to the instability of non-protected RNA, it is advantageous to provide the RNA molecules of the present invention in complexed or encapsulated form. Respective systems, in particular compositions, are provided in the present invention. In particular, in some embodiments, the system of the present invention comprises nucleic acid-containing particles, preferably RNA-containing particles. The nucleic acid-containing particles may be, for example, in the form of proteinaceous particles or in the form of lipid-containing particles. Suitable proteins or lipids are referred to as particle forming agents. Proteinaceous particles and lipid-containing particles have been described previously to be suitable for delivery of alphaviral RNA in particulate form (e.g. Strauss & Strauss, 1994, Microbiol. Rev. 58:491-562). In particular, alphavirus structural proteins (provided e.g. by a helper virus) are a suitable carrier for delivery of RNA in the form of proteinaceous particles. The system may comprise a first composition comprising the first RNA molecule, and a second composition comprising the second RNA molecule, and optionally one or more further compositions comprising any further RNA molecules (e.g., a third RNA molecule). The system may comprise a composition comprising the first RNA molecule and the second RNA molecule, and optionally any further RNA molecules (e.g., a third RNA molecule). The system may comprise a composition comprising particles comprising the first RNA molecule and particles comprising the second RNA molecule. The system may comprise a composition comprising particles comprising a mixture of the first RNA molecule and the second RNA molecule.

In one embodiment, the system according to the present invention comprises nucleic acid according to the invention in the form of nanoparticles. Nanoparticulate formulations can be obtained by various protocols and with various complexing compounds. Lipids, polymers, oligomers, or amphipiles are typical constituents of nanoparticulate formulations.

As used herein, the term “nanoparticle” refers to any particle having a diameter making the particle suitable for systemic, in particular parenteral, administration, of, in particular, nucleic acids, typically a diameter of 1000 nanometers (nm) or less. In one embodiment, the nanoparticles have an average diameter in the range of from about 50 nm to about 1000 nm, preferably from about 50 nm to about 400 nm, preferably about 100 nm to about 300 nm such as about 150 nm to about 200 nm. In one embodiment, the nanoparticles have a diameter in the range of about 200 to about 700 nm, about 200 to about 600 nm, preferably about 250 to about 550 nm, in particular about 300 to about 500 nm or about 200 to about 400 nm. In one embodiment, the average diameter is between about 50 to 150 nm, preferably, about 60 to 120 nm. In one embodiment, the average diameter is less than 50 nm.

In one embodiment, the polydispersity index (PI) of the nanoparticles described herein, as measured by dynamic light scattering, is 0.5 or less, preferably 0.4 or less or even more preferably 0.3 or less. The “polydispersity index” (PI) is a measurement of homogeneous or heterogeneous size distribution of the individual particles (such as liposomes) in a particle mixture and indicates the breadth of the particle distribution in a mixture. The PI can be determined, for example, as described in WO 2013/143555 A1.

As used herein, the term “nanoparticulate formulation” or “nanoparticulate system” or similar terms refer to any system, in particular composition, that contains at least one nanoparticle. In some embodiments, a nanoparticulate system is a uniform collection of nanoparticles. In some embodiments, a nanoparticulate system is a lipid-containing system, such as a liposome formulation or an emulsion.

### Lipid-Containing Systems

In one embodiment, the system of the present invention comprises at least one lipid. Preferably, at least one lipid is a cationic lipid. Said lipid-containing system comprises nucleic acid according to the present invention. In one embodiment, the system according to the invention comprises RNA encapsulated in a vesicle, e.g. in a liposome. In one embodiment, the system according to the invention comprises RNA in the form of an emulsion. In one embodiment, the system according to the invention comprises RNA in a complex with a cationic compound, thereby forming e.g. so-called lipoplexes. Encapsulation of RNA within vesicles such as liposomes is distinct from, for instance, lipid/RNA complexes. Lipid/RNA complexes are obtainable e.g. when RNA is e.g. mixed with pre-formed liposomes.

In one embodiment, the system according to the invention comprises RNA encapsulated in a vesicle. Such formulation is a particular system according to the invention. A vesicle is a lipid bilayer rolled up into a spherical shell, enclosing a small space and separating that space from the space outside the vesicle. Typically, the space inside the vesicle is an aqueous space, i.e. comprises water. Typically, the space outside the vesicle is an aqueous space, i.e. comprises water. The lipid bilayer is formed by one or more lipids (vesicle-forming lipids). The membrane enclosing the vesicle is a lamellar phase, similar to that of the plasma membrane. The vesicle according to the present invention may be a multilamellar vesicle, a unilamellar vesicle, or a mixture thereof. When encapsulated in a vesicle, the RNA is typically separated from any external medium. Thus, it is present in protected form, functionally equivalent to the protected form in a natural alphavirus. Suitable vesicles are particles, particularly nanoparticles, as described herein.

For example, RNA may be encapsulated in a liposome. In that embodiment, the system is or comprises a liposome formulation. Encapsulation within a liposome will typically protect RNA from RNase digestion. It is possible that the liposomes include some external RNA (e.g. on their surface), but at least half of the RNA (and ideally all of it) is encapsulated within the core of the liposome.

Liposomes are microscopic lipidic vesicles often having one or more bilayers of a vesicle-forming lipid, such as a phospholipid, and are capable of encapsulating a drug, e.g. RNA. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. Such phases may be present in nanoparticulate RNA formulations of the present invention.

Liposomes may be formed using standard methods known to the skilled person. Respective methods include the reverse evaporation method, the ethanol injection method, the dehydration-rehydration method, sonication or other suitable methods. Following liposome formation, the liposomes can be sized to obtain a population of liposomes having a substantially homogeneous size range.

In a preferred embodiment of the present invention, the RNA is present in a liposome which includes at least one cationic lipid. Respective liposomes can be formed from a single lipid or from a mixture of lipids, provided that at least one cationic lipid is used. Preferred cationic lipids have a nitrogen atom which is capable of being protonated; preferably, such cationic lipids are lipids with a tertiary amine group. A particularly suitable lipid with a tertiary amine group is 1,2-dilinoleyloxy-N,N-dimethyl-3-aminopropane (DLinDMA). In one embodiment, the RNA according to the present invention is present in a liposome formulation as described in WO 2012/006378 A1: a liposome having a lipid bilayer encapsulating an aqueous core including RNA, wherein the lipid bilayer comprises a lipid with a pKa in the range of 5.0 to 7.6, which preferably has a tertiary amine group. Preferred cationic lipids with a tertiary amine group include DLinDMA (pKa 5.8) and are generally described in WO 2012/031046 A2. According to WO 2012/031046 A2, liposomes comprising a respective compound are particularly suitable for encapsulation of RNA and thus liposomal delivery of RNA. In one embodiment, the RNA according to the present invention is present in a liposome formulation, wherein the liposome includes at least one cationic lipid whose head group includes at least one nitrogen atom (N) which is capable of being protonated, wherein the liposome and the RNA have a N:P ratio of between 1:1 and 20:1. According to the present invention, “N:P ratio” refers to the molar ratio of nitrogen atoms (N) in the cationic lipid to phosphate atoms (P) in the RNA comprised in a lipid containing particle (e.g. liposome), as described in WO 2013/006825 A1. The N:P ratio of between 1:1 and 20:1 is implicated in the net charge of the liposome and in efficiency of delivery of RNA to a vertebrate cell.

In one embodiment, the RNA according to the present invention is present in a liposome formulation that comprises at least one lipid which includes a polyethylene glycol (PEG) moiety, wherein RNA is encapsulated within a PEGylated liposome such that the PEG moiety is present on the liposome's exterior, as described in WO 2012/031043 A1 and WO 2013/033563 A1.

In one embodiment, the RNA according to the invention is not present in a liposome formulation comprises at least one lipid which includes a polyethylene glycol (PEG) moiety.

In one embodiment, the RNA according to the present invention is present in a liposome formulation, wherein the liposome has a diameter in the range of 60-180 nm, as described in WO 2012/030901 A1.

In one embodiment, the RNA according to the present invention is present in a liposome formulation, wherein the RNA-containing liposomes have a net charge close to zero or negative, as disclosed in WO 2013/143555 A1.

In other embodiments, the system according to the invention comprises RNA in the format of an emulsion. Emulsions have been previously described to be used for delivery of nucleic acid molecules, such as RNA molecules, to cells. Preferred herein are oil-in-water emulsions. The respective emulsion particles comprise an oil core and a cationic lipid. More preferred are cationic oil-in-water emulsions in which the RNA according to the present invention is complexed to the emulsion particles. The emulsion particles comprise an oil core and a cationic lipid. The cationic lipid can interact with the negatively charged RNA, thereby anchoring the RNA to the emulsion particles. In an oil-in-water emulsion, emulsion particles are dispersed in an aqueous continuous phase. For example, the average diameter of the emulsion particles may typically be from about 80 nm to 180 nm. In one embodiment, the system of the present invention is a cationic oil-in-water emulsion, wherein the emulsion particles comprise an oil core and a cationic lipid, as described in WO 2012/006380 A2. The RNA according to the present invention may be present in the form of an emulsion comprising a cationic lipid wherein the N:P ratio of the emulsion is at least 4:1, as described in WO 2013/006834 A1. The RNA according to the present invention may be present in the form of a cationic lipid emulsion, as described in WO 2013/006837 A1. In particular, the composition may comprise RNA complexed with a particle of a cationic oil-in-water emulsion, wherein the ratio of oil/lipid is at least about 8:1 (mole:mole).

In other embodiments, the system according to the invention comprises RNA in the format of a lipoplex. The term, “lipoplex” or “RNA lipoplex” refers to a complex of lipids and nucleic acids such as RNA. Lipoplexes can be formed of cationic (positively charged) liposomes and the anionic (negatively charged) nucleic acid. The cationic liposomes can also include a neutral “helper” lipid. In the simplest case, the lipoplexes form spontaneously by mixing the nucleic acid with the liposomes with a certain mixing protocol, however various other protocols may be applied. It is understood that electrostatic interactions between positively charged liposomes and negatively charged nucleic acid are the driving force for the lipoplex formation (WO 2013/143555 A1). In one embodiment of the present invention, the net charge of the RNA lipoplex particles is close to zero or negative. It is known that electro-neutral or negatively charged lipoplexes of RNA and liposomes lead to substantial RNA expression in spleen dendritic cells (DCs) after systemic administration and are not associated with the elevated toxicity that has been reported for positively charged liposomes and lipoplexes (cf. WO 2013/143555 A1). Therefore, in one embodiment of the present invention, the system according to the invention comprises RNA in the format of nanoparticles, preferably lipoplex nanoparticles, in which (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is 1.4:1 or less and/or (iv) the zeta potential of the nanoparticles is 0 or less. As described in WO 2013/143555 A1, zeta potential is a scientific term for electrokinetic potential in colloidal systems. In the present invention, (a) the zeta potential and (b) the charge ratio of the cationic lipid to the RNA in the nanoparticles can both be calculated as disclosed in WO 2013/143555 A1. In summary, systems which are nanoparticulate lipoplex formulations with a defined particle size, wherein the net charge of the particles is close to zero or negative, as disclosed in WO 2013/143555 A1, are preferred systems in the context of the present invention.

In other embodiments, the lipoplexes are obtained according to a method as disclosed in WO 2019/077053 A1. According to WO 2019/077053 A1, lipoplexes can be obtained by adding liposome colloid with a solution comprising RNA. The liposome colloid, according to WO 2019/077053 A1, can be obtained by a method comprising injecting a lipid solution in ethanol into an aqueous phase to produce the liposome colloid, wherein the concentration of at least one of the lipids in the lipid solution corresponds to or is higher than the equilibrium solubility of the at least one lipid in ethanol. A particularly preferred method of producing a liposome colloid comprises injecting a lipid solution comprising DOTMA and DOPE in a molar ratio of about 2:1 in ethanol into water stirred at a stirring velocity of about 150 rpm to produce the liposome colloid, wherein the concentration of DOTMA and DOPE in the lipid solution is about 330 mM.

In other embodiments, the lipoplexes are RNA lipoplex particles according to WO 2020/069632 A1 comprising RNA, and at least one cationic lipid and at least one additional lipid, sodium chloride at a concentration of about 10 mM or less, a stabilizer at a concentration of more than about 10% weight by volume percent (% w/v) and less than about 15% weight by volume percent (% w/v), and a buffer. Preferably the lipoplexes according to the invention are RNA lipoplex particles comprising DOTMA and DOPE in a molar ratio of about 2:1, wherein the ratio of positive charges to negative charges in the composition is about 1.3:2.0, sodium chloride at a concentration of about 8.2 mM, sucrose at a concentration of about 13% (w/v), HEPES at a concentration of about 5 mM with a pH of about 6.7, and EDTA at a concentration of about 2.5 mM, as described in WO 2020/069632 A1.

In one embodiment, nucleic acid such as the RNA described herein is in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP does not comprise a pegylated lipid.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure.

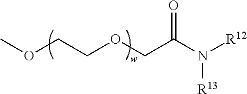

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is DMG-PEG 2000, e.g., having the following structure:

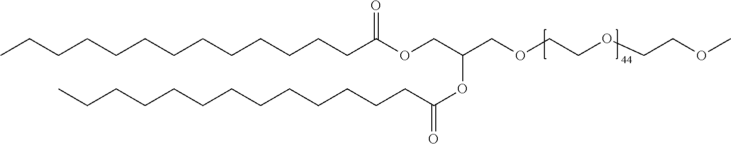

In some embodiments, the polymer conjugated lipid is not a pegylated lipid.

In some embodiments, the cationic lipid component of the LNPs has the structure of Formula (III):

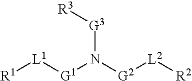

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S (O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

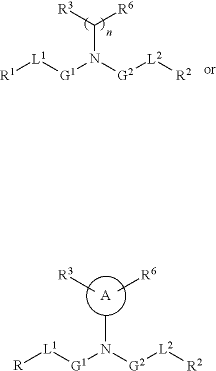

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

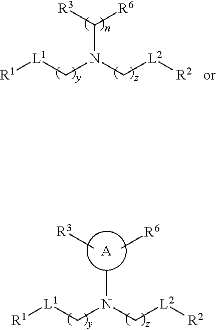

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently-(C═O)O— or —O(C═O)—. For example, in some embodiments each of L¹ and L² is —(C═O)O—.

In some different embodiments of Formula (III), the lipid has one of the following structures (IIIE) or (IIIF):

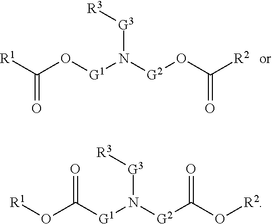

In some of the foregoing embodiments of Formula (III), the lipid has one of the following structures (IIIG), (IIIH), (IIII), or (IIIJ):

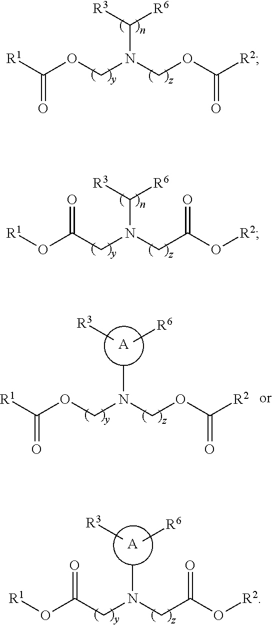

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

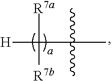

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

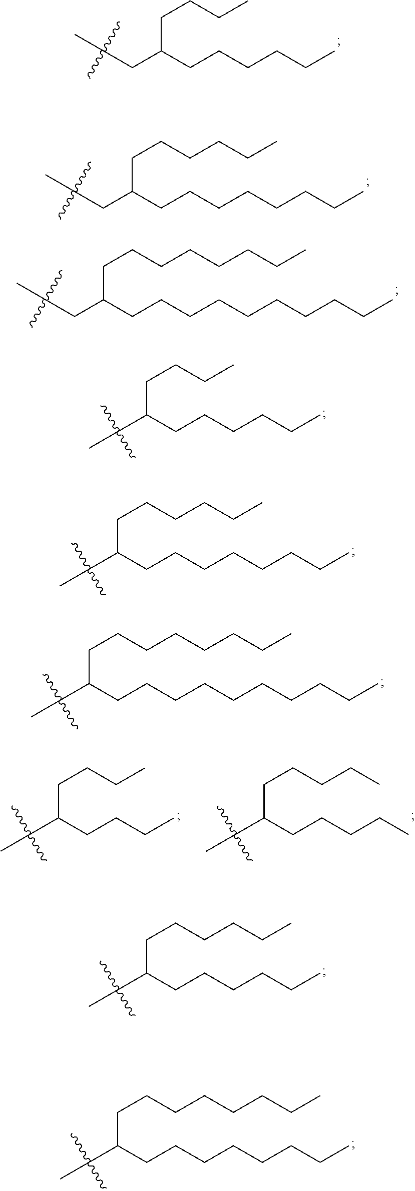

In some of the foregoing embodiments of Formula (III), R³ is OH, CN, —C(═O) OR⁴, —OC(═O)R⁴ or-NHC(═O)R⁴. In some embodiments, R⁴ is methyl or ethyl.

In various different embodiments, the cationic lipid of Formula (III) has one of the structures set forth in the table below.

### Representative Compounds of Formula (III).

| No. | Structure |
|---|---|
| III-1 | 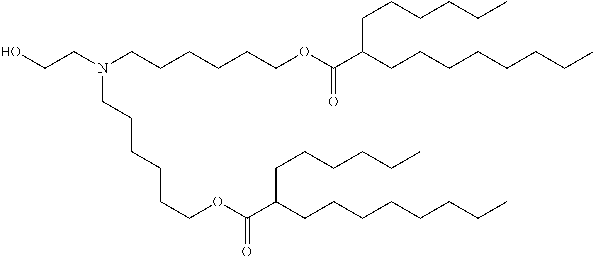 [SMILES: CCCCCCCCC(CCCCCC)C(=O)OCCCCCCN(CCO)CCCCCCOC(=O)C(CCCCCC)CCCCCCCC] |
| III-2 | 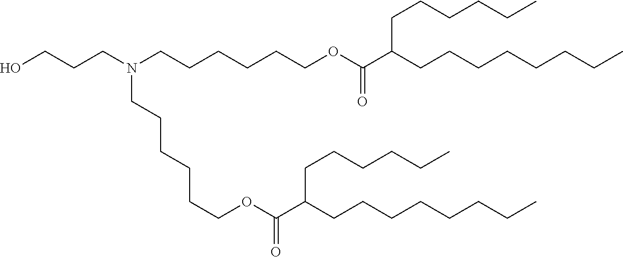 [SMILES: CCCCCCCCC(CCCCCC)C(=O)OCCCCCCN(CCCO)CCCCCCOC(=O)C(CCCCCC)CCCCCCCC] |
| III-3 | 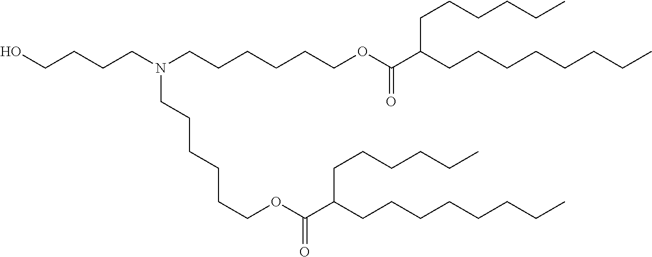 [SMILES: CCCCCCCCC(CCCCCC)C(=O)OCCCCCCN(CCCCO)CCCCCCOC(=O)C(CCCCCC)CCCCCCCC] |
| III-4 | 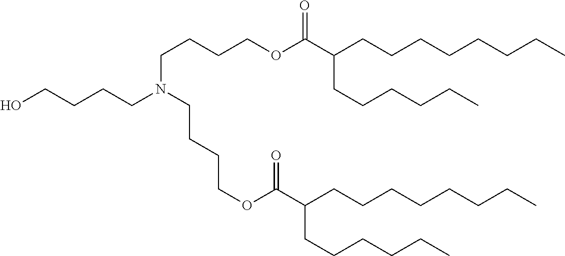 [SMILES: CCCCCCCCC(CCCCCC)C(=O)OCCCCN(CCCCO)CCCCOC(=O)C(CCCCCC)CCCCCCCC] |
| III-5 | 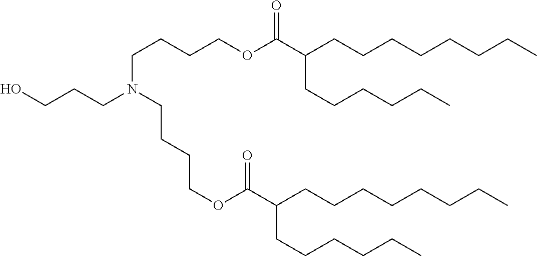 [SMILES: CCCCCCCCC(CCCCCC)C(=O)OCCCCN(CCCO)CCCCOC(=O)C(CCCCCC)CCCCCCCC] |
| III-6 | 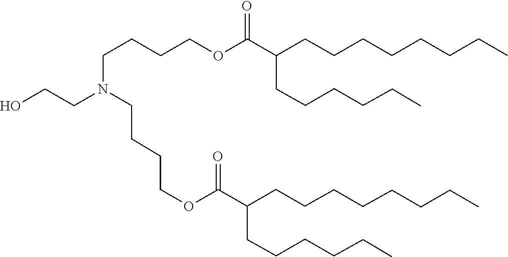 [SMILES: CCCCCCCCC(CCCCCC)C(=O)OCCCCN(CCO)CCCCOC(=O)C(CCCCCC)CCCCCCCC] |
| III-7 | 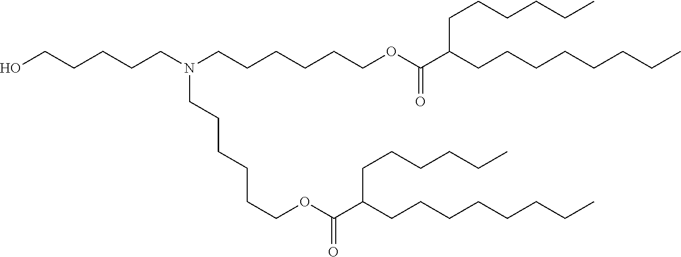 [SMILES: CCCCCCCCC(CCCCCC)C(=O)OCCCCCCN(CCCCCO)CCCCCCOC(=O)C(CCCCCC)CCCCCCCC] |
| III-8 | 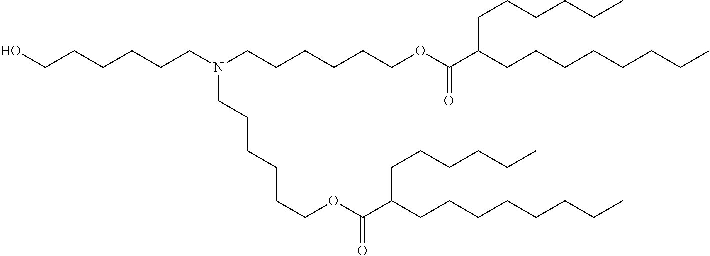 [SMILES: CCCCCCCCC(CCCCCC)C(=O)OCCCCCCN(CCCCCCO)CCCCCCOC(=O)C(CCCCCC)CCCCCCCC] |
| III-9 | 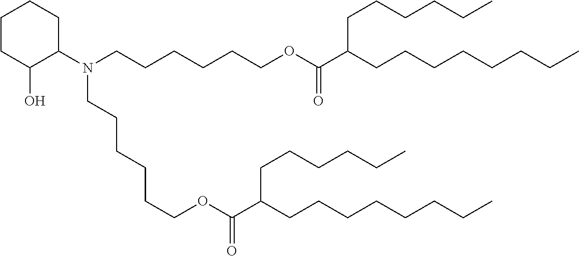 [SMILES: CCCCCCCCC(CCCCCC)C(=O)OCCCCCCN(CCCCCCOC(=O)C(CCCCCC)CCCCCCCC)C1CCCCC1O] |
| III-10 | 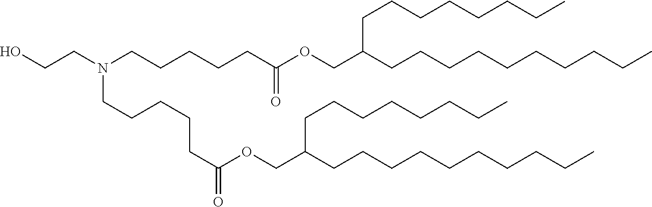 [SMILES: CCCCCCCCCCC(CCCCCCCC)COC(=O)CCCCCN(CCO)CCCCCC(=O)OCC(CCCCCCCC)CCCCCCCCCC] |
| III-11 | 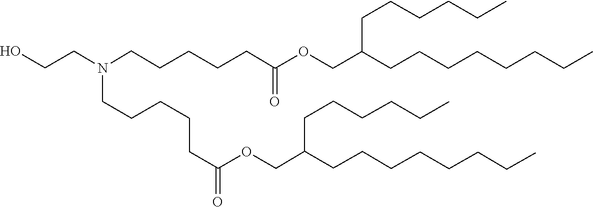 [SMILES: CCCCCCCCC(CCCCCC)COC(=O)CCCCCN(CCO)CCCCCC(=O)OCC(CCCCCC)CCCCCCCC] |
| III-12 | 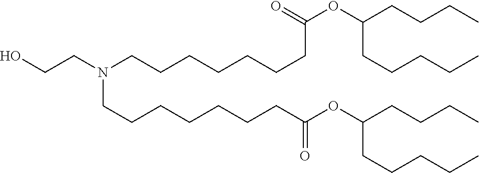 [SMILES: CCCCCC(CCCC)OC(=O)CCCCCCCN(CCO)CCCCCCCC(=O)OC(CCCC)CCCCC] |
| III-13 | 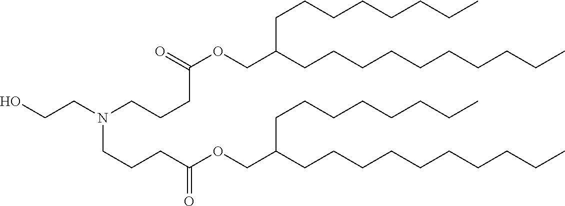 [SMILES: CCCCCCCCCCC(CCCCCCCC)COC(=O)CCCN(CCO)CCCC(=O)OCC(CCCCCCCC)CCCCCCCCCC] |
| III-14 | 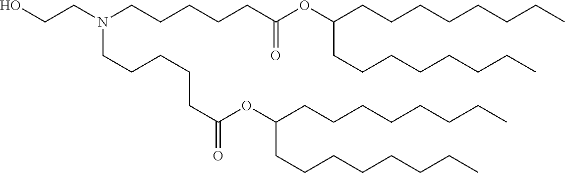 [SMILES: CCCCCCCCC(CCCCCCCC)OC(=O)CCCCCN(CCO)CCCCCC(=O)OC(CCCCCCCC)CCCCCCCC] |
| III-15 | 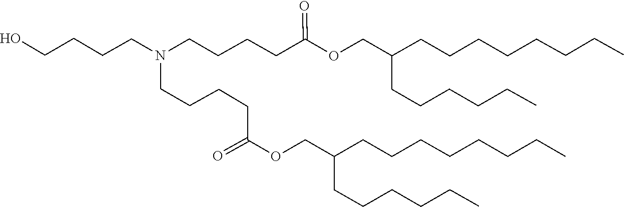 [SMILES: CCCCCCCCC(CCCCCC)COC(=O)CCCCN(CCCCO)CCCCC(=O)OCC(CCCCCC)CCCCCCCC] |
| III-16 | 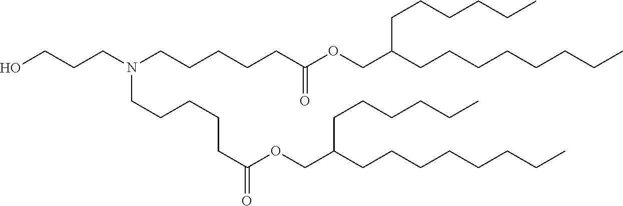 [SMILES: CCCCCCCCC(CCCCCC)COC(=O)CCCCCN(CCCO)CCCCCC(=O)OCC(CCCCCC)CCCCCCCC] |
| III-17 | 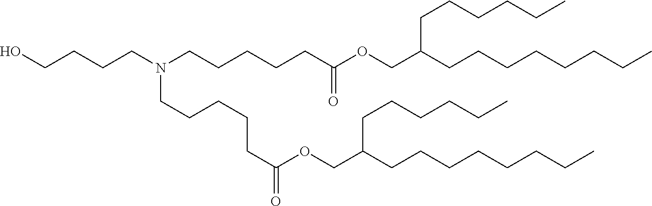 [SMILES: CCCCCCCCC(CCCCCC)COC(=O)CCCCCN(CCCCO)CCCCCC(=O)OCC(CCCCCC)CCCCCCCC] |
| III-18 | 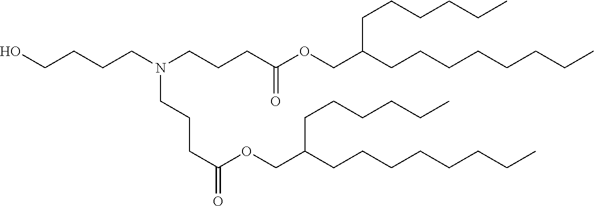 [SMILES: CCCCCCCCC(CCCCCC)COC(=O)CCCN(CCCCO)CCCC(=O)OCC(CCCCCC)CCCCCCCC] |
| III-19 | 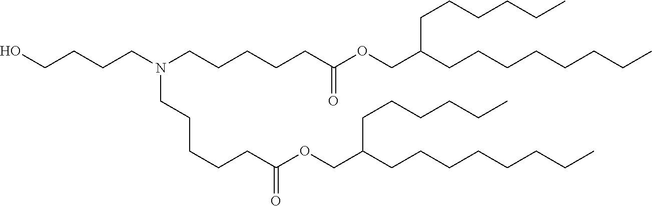 [SMILES: CCCCCCCCC(CCCCCC)COC(=O)CCCCCN(CCCCO)CCCCCC(=O)OCC(CCCCCC)CCCCCCCC] |
| III-20 | 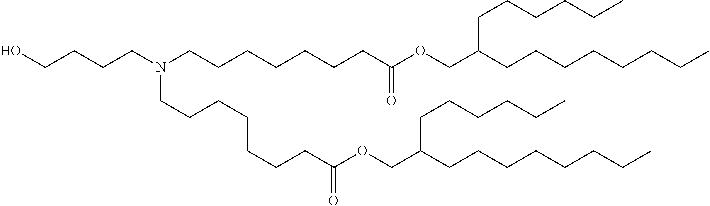 [SMILES: CCCCCCCCC(CCCCCC)COC(=O)CCCCCCCN(CCCCO)CCCCCCCC(=O)OCC(CCCCCC)CCCCCCCC] |
| III-21 | 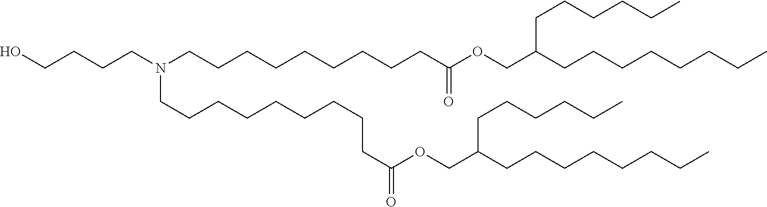 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 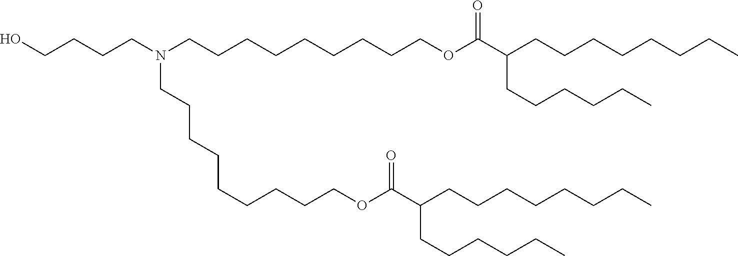 [SMILES: CCCCCCCCC(CCCCCC)C(=O)OCCCCCCCCCN(CCCCO)CCCCCCCCCOC(=O)C(CCCCCC)CCCCCCCC] |
| III-23 | 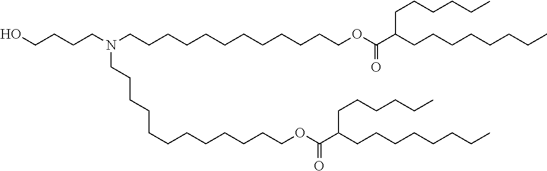 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 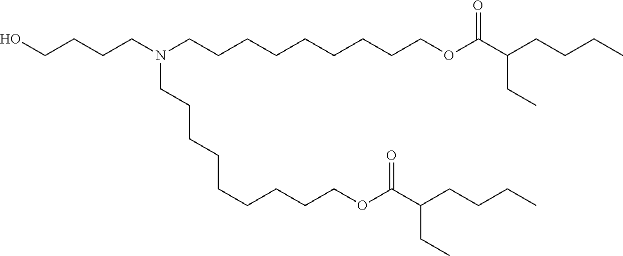 [SMILES: CCCCC(CC)C(=O)OCCCCCCCCCN(CCCCO)CCCCCCCCCOC(=O)C(CC)CCCC] |
| III-25 | 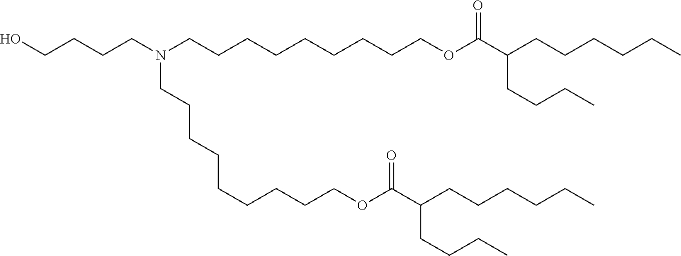 [SMILES: CCCCCCC(CCCC)C(=O)OCCCCCCCCCN(CCCCO)CCCCCCCCCOC(=O)C(CCCC)CCCCCC] |
| III-26 | 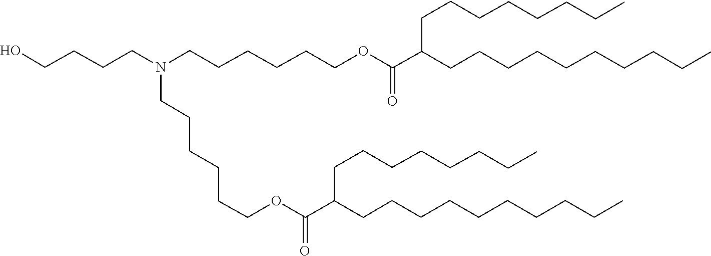 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 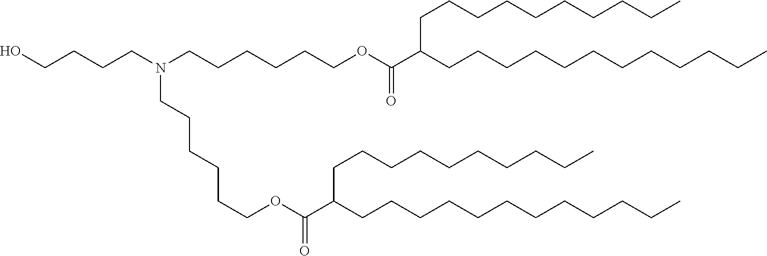 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 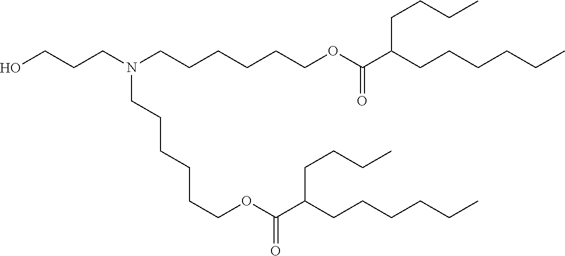 [SMILES: CCCCCCC(CCCC)C(=O)OCCCCCCN(CCCO)CCCCCCOC(=O)C(CCCC)CCCCCC] |
| III-29 | 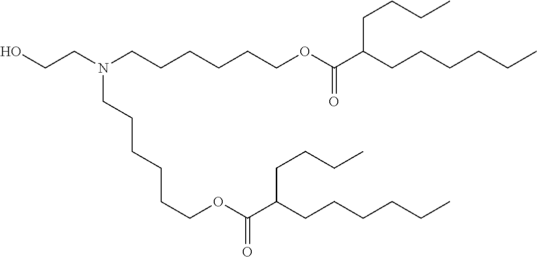 [SMILES: CCCCCCC(CCCC)C(=O)OCCCCCCN(CCO)CCCCCCOC(=O)C(CCCC)CCCCCC] |
| III-30 | 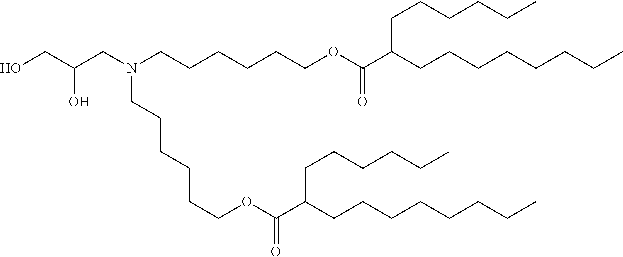 [SMILES: CCCCCCCCC(CCCCCC)C(=O)OCCCCCCN(CCCCCCOC(=O)C(CCCCCC)CCCCCCCC)CC(O)CO] |
| III-31 | 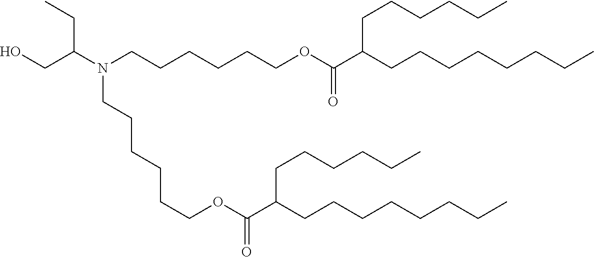 [SMILES: CCCCCCCCC(CCCCCC)C(=O)OCCCCCCN(CCCCCCOC(=O)C(CCCCCC)CCCCCCCC)C(CC)CO] |
| III-32 | 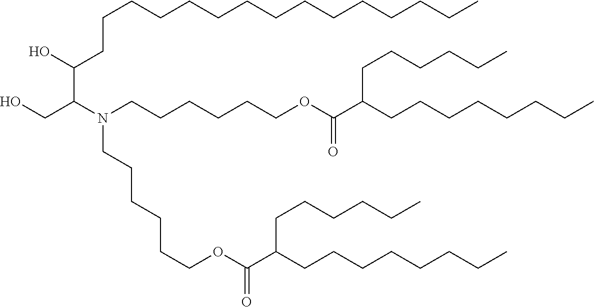 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 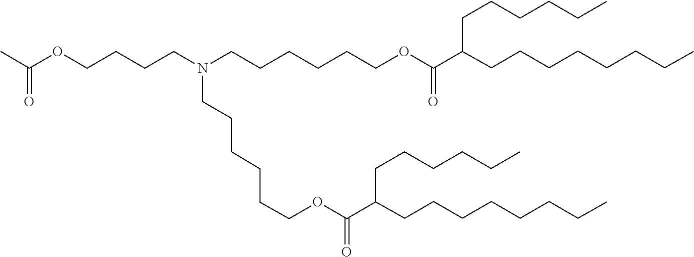 [SMILES: CCCCCCCCC(CCCCCC)C(=O)OCCCCCCN(CCCCCCOC(=O)C(CCCCCC)CCCCCCCC)CCCCOC(C)=O] |
| III-34 | 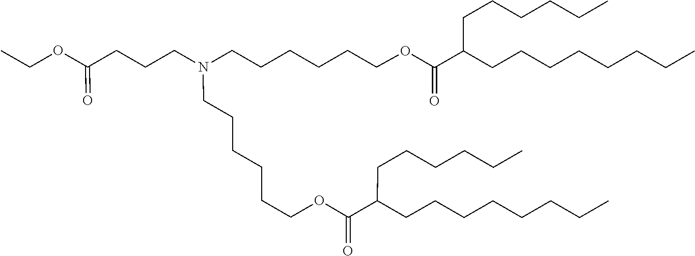 [SMILES: CCCCCCCCC(CCCCCC)C(=O)OCCCCCCN(CCCCCCOC(=O)C(CCCCCC)CCCCCCCC)CCCC(=O)OCC] |
| III-35 | 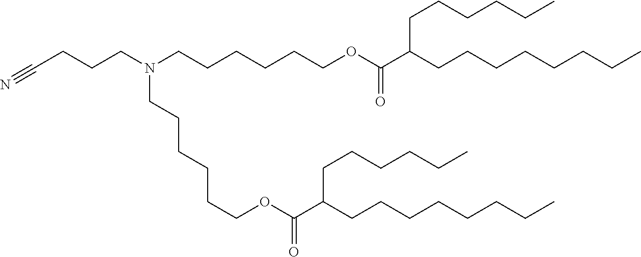 [SMILES: CCCCCCCCC(CCCCCC)C(=O)OCCCCCCN(CCCC#N)CCCCCCOC(=O)C(CCCCCC)CCCCCCCC] |
| III-36 | 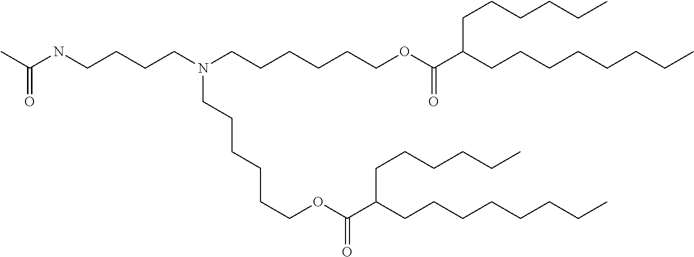 [SMILES: CCCCCCCCC(CCCCCC)C(=O)OCCCCCCN(CCCCCCOC(=O)C(CCCCCC)CCCCCCCC)CCCCNC(C)=O] |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159. In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent. In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

| No. | Structure |
|---|---|
| A | 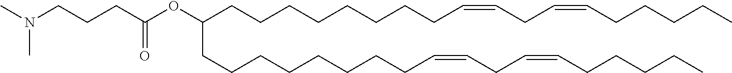 [SMILES: CCCCC/C=C\C/C=C\CCCCCCCCC(CCCCCCCC/C=C\C/C=C\CCCCC)OC(=O)CCCN(C)C] |
| B | 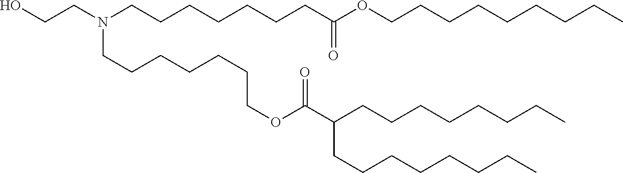 [SMILES: CCCCCCCCCOC(=O)CCCCCCCN(CCO)CCCCCCCOC(=O)C(CCCCCCCC)CCCCCCCC] |
| C | 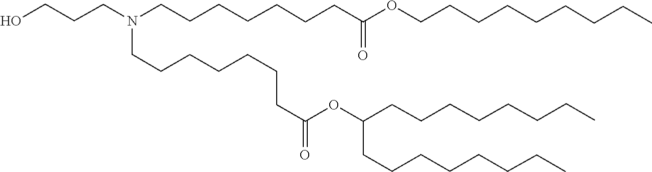 [SMILES: CCCCCCCCCOC(=O)CCCCCCCN(CCCO)CCCCCCCC(=O)OC(CCCCCCCC)CCCCCCCC] |
| D | 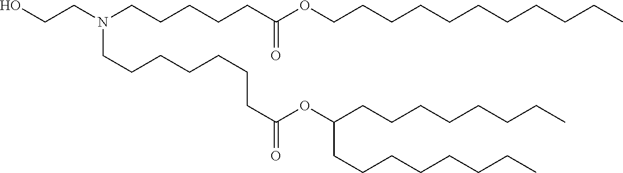 [SMILES: CCCCCCCCCCCOC(=O)CCCCCN(CCO)CCCCCCCC(=O)OC(CCCCCCCC)CCCCCCCC] |
| E | 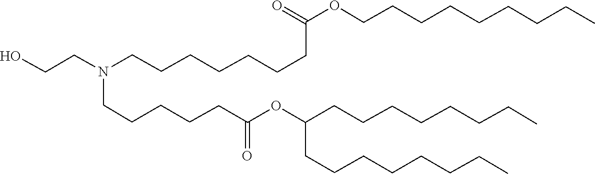 [SMILES: CCCCCCCCCOC(=O)CCCCCCCN(CCO)CCCCCC(=O)OC(CCCCCCCC)CCCCCCCC] |
| F | 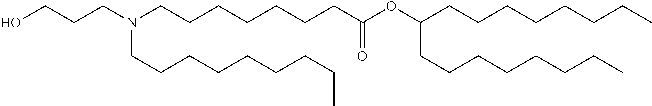 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In one embodiment, the LNP comprises a cationic lipid that is an ionizable lipid-like material (lipidoid). In one embodiment, the cationic lipid has the following structure:

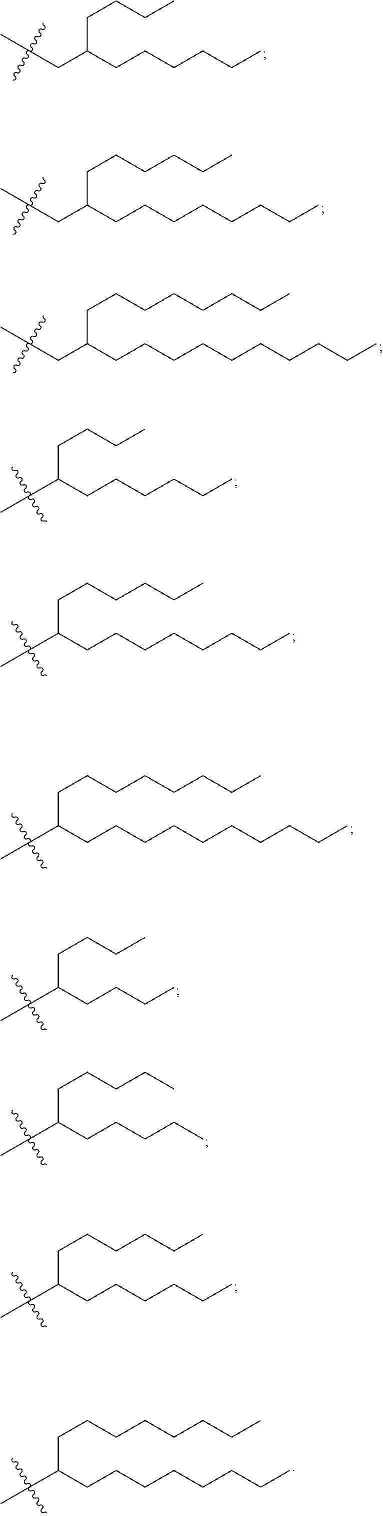

The N/P value is preferably at least about 4. In some embodiments, the N/P value ranges from 4 to 20, 4 to 12, 4 to 10, 4 to 8, or 5 to 7. In one embodiment, the N/P value is about 6.

LNP described herein may have an average diameter that in one embodiment ranges from about 30 nm to about 200 nm, or from about 60 nm to about 120 nm.

### RNA Targeting

Some aspects of the disclosure involve the targeted delivery of the RNA disclosed herein (e.g., RNA encoding vaccine antigens and/or immunostimulants).

In one embodiment, the disclosure involves targeting lung. Targeting lung is in particular preferred if the RNA administered is RNA encoding vaccine antigen or a miRNA relevant in the treatment of an infectious disease in the lungs. RNA may be delivered to lung, for example, by administering the RNA which may be formulated as particles as described herein, e.g., lipid particles, by inhalation.

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2, or about 1.6:2 to about 1:2, or about 1.6:2 to about 1.1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Immunostimulants may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

Examples of suitable immunostimulants for targeting liver are cytokines involved in T cell proliferation and/or maintenance. Examples of suitable cytokines include IL2 or IL7, fragments and variants thereof, and fusion proteins of these cytokines, fragments and variants, such as extended-PK cytokines.

In another embodiment, RNA encoding an immunostimulant may be administered in a formulation for preferential delivery of RNA to the lymphatic system, in particular secondary lymphoid organs, more specifically spleen. The delivery of an immunostimulant to such target tissue is preferred, in particular, if presence of the immunostimulant in this organ or tissue is desired (e.g., for inducing an immune response, in particular in case immunostimulants such as cytokines are required during T-cell priming or for activation of resident immune cells), while it is not desired that the immunostimulant is present systemically, in particular in significant amounts (e.g., because the immunostimulant has systemic toxicity).

Examples of suitable immunostimulants are cytokines involved in T cell priming. Examples of suitable cytokines include IL12, IL15, IFN-α, or IFN-β, fragments and variants thereof, and fusion proteins of these cytokines, fragments and variants, such as extended-PK cytokines.

### Polymer-Based Systems

In one embodiment, the system of the present invention comprises at least one polymer, preferably a polyalkyleneimine.

In some embodiments, the particles formed from the RNA and the polymer, preferably polyaklyeneimine, are polymer-based polyplexes.

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethylenimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropyleneimine, preferably polyethylenimine. A preferred polyalkyleneimine is polyethylenimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethylenimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

In an embodiment, the system comprise polyalkyleneimine, and, for example, the molar ratio of the number of nitrogen atoms (N) in the polyalkyleneimine to the number of phosphor atoms (P) in the RNA molecules (N:P ratio) can be 2.0 to 15.0, preferably 6.0 to 12.0 and/or the ionic strength of the composition can be 50 mM or less, preferably wherein the concentration of monovalent cationic ions can be 25 mM or less and the concentration of divalent cationic ions can be 20 UM or less.

In an embodiment, the particles formed can be polyplexes.

In an embodiment, the polyalkyleneimine can comprise the following general formula (I):

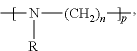

wherein
R is H, an acyl group or a group comprising the following general formula (II):

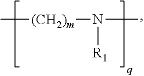

wherein R¹ is H or a group comprising the following general formula (III):

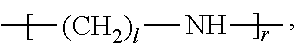

n, m, and l are independently selected from integers from 2 to 10; and
p, q, and r are integers, wherein the sum of p, q, and r is such that the average molecular weight of the polymer is 1.5.10² to 10⁷ Da, preferably 5000 to 10⁵ Da, more preferably 10000 to 40000 Da, more preferably 15000 to 30000 Da, even more preferably 20000 to 25000 Da. In an embodiment, n, m, and l can be independently selected from 2, 3, 4, and 5, preferably from 2 and 3 and/or R₁ can be H. In an embodiment, R can be H or an acyl group.

In an embodiment, the polyalkyleneimine can comprise polyethylenimine and/or polypropylenimine, preferably polyethylenimine. In an embodiment, at least 92% of the N atoms in the polyalkyleneimine can be protonatable.

### Kit

The present invention also provides a kit comprising the at least two RNA molecules according to the invention.

In one embodiment, the constituents of the kit are present as separate entities. For example, one constituent of the kit may be present in one entity, and another constituent of the kit may be present in a separate entity. For example, an open or closed container is a suitable entity. A closed container is preferred. The container used should preferably be RNAse-free or essentially RNAse-free.

In one embodiment, the kit of the present invention comprises RNA for inoculation with a cell and/or for administration to a human or animal subject.

The kit according to the present invention optionally comprises a label or other form of information element, e.g. an electronic data carrier. The label or information element preferably comprises instructions, e.g. printed written instructions or instructions in electronic form that are optionally printable. The instructions may refer to at least one suitable possible use of the kit.

### Pharmaceutical Composition

A pharmaceutical composition according to the invention may comprise at least one nucleic acid molecule according to the present invention. A pharmaceutical composition according to the invention comprises a pharmaceutically acceptable diluent and/or a pharmaceutically acceptable excipient and/or a pharmaceutically acceptable carrier and/or a pharmaceutically acceptable vehicle. The choice of pharmaceutically acceptable carrier, vehicle, excipient or diluent is not particularly limited. Any suitable pharmaceutically acceptable carrier, vehicle, excipient or diluent known in the art may be used.

In one embodiment of the present invention, a pharmaceutical composition can further comprise a solvent such as an aqueous solvent or any solvent that makes it possible to preserve the integrity of the RNA. In a preferred embodiment, the pharmaceutical composition is an aqueous solution comprising RNA. The aqueous solution may optionally comprise solutes, e.g. salts.

In one embodiment of the present invention, the pharmaceutical composition is in the form of a freeze-dried composition. A freeze-dried composition is obtainable by freeze-drying a respective aqueous composition.

In some embodiments, the pharmaceutical compositions according to the invention are for use in the manufacture of a medicament for the treatment or prevention of a disease, preferably for a method of treatment as described herein.

### Medicaments

In view of the capacity to be administered to a subject, each of the RNA molecules according to the invention, the system according to the invention or the kit according to the invention, or the pharmaceutical composition according to the invention, may be referred to as “medicament”, a “medical preparation” or the like. The present invention foresees that the first RNA molecule, the second RNA molecule the kit, the pharmaceutical composition or the system of the present invention is provided for use as a medicament. The medicament can be used to treat a subject. By “treat” is meant to administer a compound or composition or other entity as described herein to a subject. The term includes methods for treatment of the human or animal body by therapy.

The above described medicament does typically not comprise a DNA, and is thus associated with additional safety features compared to DNA based medicaments, e.g., vaccines, described in the prior art (e.g. WO 2008/119827 A1).

The medicament according to the present invention may be administered to a subject in need thereof. The medicament of the present invention can be used in prophylactic as well as in therapeutic methods of treatment of a subject.

The medicament according to the invention is administered in an effective amount. An “effective amount” concerns an amount that is sufficient, alone or together with other doses, to cause a reaction or a desired effect. In the case of treatment of a certain disease or a certain condition in a subject, the desired effect is the inhibition of disease progression. This includes the deceleration of disease progression, in particular the interruption of disease progression. The desired effect in the treatment of a disease or a condition can also be a delay of disease outbreak or the inhibition of disease outbreak.

The effective amount will depend on the condition being treated, the severity of the disease, the individual parameters of the patient, including age, physiological condition, size and weight, duration of the treatment, type of accompanying therapy (if any), the specific mode of administration and other factors.

### Methods of Treatment

In an embodiment, the methods for the treatment or prevention of a bacterial, viral, parasitical or fungal infection in a subject, said method comprising administering to the subject a pharmaceutical composition as described herein.

In an embodiment, the methods for the treatment or prevention of cancer in a subject, said method comprising administering to the subject a pharmaceutical composition as described herein.

In an embodiment, the methods for treatment described herein are vaccinations, in particular against infectious diseases, such as by a bacterium, virus, fungus or parasite, or cancer.

Also described herein is a first RNA molecule and a second RNA molecule, as described herein, for use in a method for (i) the treatment or prevention of a bacterial, viral, parasitical or fungal infection, (ii) the treatment or prevention of cancer, or (ii) vaccination, in particular against infectious diseases, such as by a bacterium, virus, fungus or parasite, or cancer, in a subject; said method comprising administering to the subject the first RNA molecule and the second RNA molecule. Also described herein is a first RNA molecule, as described herein, for use in a method of treatment in a subject as described herein, said method comprising administering to the subject the first RNA molecule, wherein the subject is or has been also administered a second RNA molecule as described herein. Also described herein is a second RNA molecule, as described herein, for use in a method of treatment in a subject as described herein, said method comprising administering to the subject the second RNA molecule, wherein the subject is or has been also administered a first RNA molecule as described herein.

The term “immunization” or “vaccination” generally refers to a process of treating a subject for therapeutic or prophylactic reasons. A treatment, particularly a prophylactic treatment, is or comprises preferably a treatment aiming to induce or enhance an immune response of a subject, e.g. against one or more antigens. If, according to the present invention, it is desired to induce or enhance an immune response by using RNA as described herein, the immune response may be triggered or enhanced by the RNA. In one embodiment, the invention provides a prophylactic treatment which is or comprises preferably the vaccination of a subject. An embodiment of the present invention wherein the replicon comprises at least one miRNA and encodes, as a protein of interest, a pharmaceutically active peptide or protein which is an immunologically active compound or an antigen is particularly useful for vaccination.

RNA has been previously described for vaccination against foreign agents including pathogens or cancer (reviewed recently by Ulmer et al., 2012, Vaccine 30:4414-4418). In contrast to common approaches in the prior art, the replicon according to the present invention is a particularly suitable element for efficient treatment or prevention, in particular vaccination, because of the ability to be replicated by functional alphavirus non-structural protein as described herein. The treatment or prevention, in particular vaccination, according to the present invention can be used for example for induction of an immune response to weakly immunogenic proteins. In the case of the RNA vaccines according to the invention, the protein antigen is never exposed to serum antibodies, but is produced by transfected cells themselves after translation of the RNA. Therefore, anaphylaxis should not be a problem. The invention therefore permits the repeated immunization of a patient without risk of allergic reactions.

In methods involving treatment or prevention, in particular vaccination, according to the present invention, the medicament of the present invention is administered to a subject, in particular if treating a subject having a disease involving the antigen or at risk of falling ill with the disease involving the antigen is desired.

In methods involving treatment or prevention according to the present invention, the miRNA and optionally protein of interest encoded by the replicon according to the present invention codes for example for a miRNA beneficial for the treatment or prevention of a bacterial infection, a viral infection, fungal infection or cancer and optionally a bacterial antigen, against which an immune response is to be directed, or for a viral antigen, against which an immune response is to be directed, or for a cancer antigen, against which an immune response is to be directed, or for an antigen of a unicellular organism, against which an immune response is to be directed. The efficacy of treatment, in particular vaccination, can be assessed by known standard methods such as by measurement of antigen-specific IgG antibodies from the organism. In methods involving allergen-specific immunotherapy according to the present invention, the protein of interest encoded by the replicon according to the present invention codes for an antigen relevant to an allergy. Allergen-specific immunotherapy (also known as hypo-sensitization) is defined as the administration of preferably increasing doses of an allergen vaccine to an organism with one or more allergies, in order to achieve a state in which the symptoms that are associated with a subsequent exposure to the causative allergen are alleviated. The efficacy of an allergen-specific immunotherapy can be assessed by known standard methods such as by measurement of allergen-specific IgG and IgE antibodies from the organism.

The medicament of the present invention can be administered to a subject, e.g. for treatment of the subject, including vaccination of the subject.

The term “subject” relates to vertebrates, particularly mammals. For example, mammals in the context of the present invention are humans, non-human primates, domesticated mammals such as dogs, cats, sheep, cattle, goats, pigs, horses etc., laboratory animals such as mice, rats, rabbits, guinea pigs, etc. as well as animals in captivity such as animals of zoos. The term “subject” also relates to non-mammalian vertebrates such as birds (particularly domesticated birds such as chicken, ducks, geese, turkeys) and to fish (particularly farmed fish, e.g., salmon or catfish). The term “animal” as used herein also includes humans. The subject is preferably a human, optionally a human patient.

The administration to domesticated animals such as dogs, cats, rabbits, guinea pigs, hamsters, sheep, cattle, goats, pigs, horses, chicken, ducks, geese, turkeys, or wild animals, e.g., foxes, is preferred in some embodiments. For example, a prophylactic vaccination according to the present invention may be suitable to vaccinate an animal population, e.g. in the farming industry, or a wild animal population. Other animal populations in captivity, such as pets, or animals of zoos, may be vaccinated.

In an embodiment, the medicament can be administered more than once. Multiple doses can be administered such that individual doses can be administered at different intervals. For example, a dose can be administered 14 to 35 days after the previous dose has been administered. In an embodiment, a dose is administered 21 days after the previous dose. In an embodiment, a dose is administered 35 days after the previous dose.

In an embodiment, when administered to a subject, the system used as a medicament does preferably not comprise sequences from a type of virus, e.g., alphavirus, that is infectious to the species or genus to which the treated subject belongs. Preferably, in that case, the replicon does not comprise any nucleotide sequence from an alphavirus that can infect the respective species or genus. This embodiment bears the advantage that no recombination with infectious (e.g. fully functional or wild-type) alphavirus is possible, even if the subject to which the RNA is administered is (e.g. accidentally) affected by infectious alphavirus. As an illustrative example, for treatment of pigs, the system used does not comprise any nucleotide sequence from an alphavirus that can infect pigs.

### Mode of Administration

The pharmaceutical compositions, in particular medicament, according to the present invention can be applied to a subject in any suitable route.

For example, the medicament may be administered systemically, for example intravenously (i.v.), intramuscularly (i.m.), subcutaneously (s.c.), intradermally (i.d.) or by inhalation.

In one embodiment, the system, in particular medicament, according to the present invention is administered to muscle tissue, such as skeletal muscle, or skin, e.g. subcutaneously. It is generally understood that transfer of RNA into the skin or muscles leads to high and sustained local expression, paralleled by a strong induction of humoral and cellular immune responses (Johansson et al., 2012, PLOS. One. 7: e29732; Geall et al., 2012, Proc. Natl. Acad. Sci. U.S.A 109:14604-14609).

Alternatives to administration to muscle tissue or skin include, but are not limited to: intradermal, intranasal, intraocular, intraperitoneal, intravenous, interstitial, buccal, transdermal, or sublingual administration. Intradermal and intramuscular administration are two preferred routes.

Administration can be achieved in various ways. In one embodiment, the system, in particular medicament, according to the present invention is administered by injection. In a preferred embodiment, injection is via a needle. Needle-free injection may be used as an alternative.

The present invention is described in detail and is illustrated by the figures and examples, which are used only for illustration purposes and are not meant to be limiting. Owing to the description and the examples, further embodiments which are likewise included in the invention are accessible to the skilled worker.

### DESCRIPTION OF THE FIGURES

FIG. 1: Scheme of taRNA-miR vector and miRNA structure. (A) taRNA-miR vector comprises two capped and poly(A)-tailed RNA molecules. One non-replicative mRNA coding for the VEEV replicase and its replicable template or NTR (nano-transreplicon) coding for one or multiple transgenes alongside a pre-miRNA transcript within its 3′UTR. Replicase-dependent amplification of NTR RNA leads to high-level expression of the transgene(s). (B) Sequence of the murine pri-miRNA-155 backbone (SEQ ID NO: 37 to 39) and predicted secondary structure using MFOLD (Version 2.3) of the miR-p53-2 which was incorporated into the 3′UTR of the taRNA-miR vector (SEQ ID NO: 40). The guide strand is from 14 nt to 34 nt and the passenger strand from 54 nt to 72 nt. A black solid and dashed line respectively surrounds the guide and passenger strand. Microprocessor cleavage sites are marked with black arrows. Dicer cleavage sites are marked with white arrows. CSE, (viral) conserved sequence element, UTR, untranslated region.

FIG. 2: Stable overexpression of miR-lacZ leads to efficient knock-down of β-galactosidase. (A) BHK21 cells were transduced with lacZ-encoding lentiviruses. 48 hours after transduction, luminescence-based assays were performed to measure reporter protein expression in transduced and mock cells, respectively. The graph shows mean (SD) of quintuplicates. (B,C) BHK-lacZ cells were then transduced with the lentivirus containing miR-neco (negative control) or miR-lacZ or left untransduced (mock). (B) At indicated time points after transduction the relative ß-galactosidase expression normalized to mock-treated cells were assessed (mean (SD) of n=3). Statistical analysis was a two-way ANOVA. (C) qRT-PCR-based quantification of lacZ RNA was performed with total RNAs harvested 96 h after transduction with the miR-encoding lentiviruses. Data were normalized (AACt method) to β-actin and mock-treated samples (mean (SD) of n=3). Statistical analysis was a one-way ANOVA; *, P<0.05; **, P<0.01; ***, P<0.001; ****, P<0.0001 and ns, not significant, RE, relative expression.

FIG. 3.1: Inserting miRNA into replicating RNA does not affect protein expression in cells. BHK-lacZ cells were electroporated with 2 μg of the indicated saRNA or 5 μg of taRNA (1 μg NTR-emGFP-miR) or without RNA (mock). 24 h after transfection GFP-expression was assessed by flow cytometry. (A) Forward versus side scatter (FSC and SSC) was used to gate on living BHK-lacZ cells (first upper dot blot). GFP-fluorescence of gated cells was plotted against an irrelevant channel (APC-Cy7). (B) The percentage of GFP-positive cells and (C) their MFIs were determined (mean (SD) of n=3). MFI, mean fluorescence intensity.

FIG. 3.2: Alphaviral miRNA delivery leads to knockdown of lacZ. BHK-lacZ cells were electroporated with 2 μg of the indicated saRNA or 5 μg of taRNA (1 μg NTR-emGFP-miR) or left untreated (mock). (A,B, D,E) At indicated time points after transfection, (A, D) relative cell viability and (B, E) relative β-galactosidase expression, normalized to mock-treated cells, were calculated (mean (SD) of n=3). Statistical analysis was a two-way ANOVA. (C, F) qRT-PCR-based quantification of lacZ transcripts was performed with total RNAs harvested 72 h after transfection. Data were normalized to β-actin and mock-treated samples (mean (SD) of n=3). Statistical analysis was a one-way ANOVA; *, P<0.05; **, P<0.01; ***, P<0.001; ****, P<0.0001, and ns, not significant; RE, relative expression; ß-Gal, ß-galactosidase.

FIG. 4: taRNA-miR-luc is non-cytotoxic and downregulates luciferase expression only when actively replicated. (A) BHK21 cells were transduced with luciferase-encoding lentiviruses. 48 hours after transduction, luciferase expression was measured in transduced and mock cells, respectively. The graph shows mean (SD) of quintuplicates. (B-E) BHK-luc cells were electroporated with 0.5 μg of the indicated NTR constructs alone (-replicase), or co-delivered with 1 μg replicase mRNA (+replicase). Controls were left untransfected (mock). (B, D) At indicated time points after transfection relative cell viability normalized to mock-treated cells were calculated (mean (SD) of n=3). (C, E) At indicated time points after transfection relative luciferase expression normalized to mock-treated cells were calculated (mean (SD) of n=3). Statistical analysis was a two-way ANOVA. **, P<0.01; **, P<0.0001, and ns, not significant; MFI, mean fluorescence intensity; luc, luciferase.

FIG. 5: taRNA-miR against TP53 downregulates endogenous p53 expression. (A-C) HDFn cells were electroporated with the indicated taRNA-miR constructs (0.787 μmol/RNA) and 0.5 μg E3 mRNA and 0.5 μg B18R mRNA. Control cells were electroporated without RNA (mock). For siRNA transfection, cells were lipofected with 30 nM of a TP53 targeting pool of 3-5 different siRNAs, or 30 nM of a scrambled siRNA control. 72 h after transfection cells were pelleted, lysed and prepared for (A) western blot analysis of p53 protein and (B) relative TP53 gene expression by RT-qPCR. Data are shown as means after normalization to a reference gene and taRNA-miR-neco transfected cells (mean (SD) of n=3). Protein levels were quantified by densitometry and normalized by the signal of housekeeping protein GAPDH in three independent experiments. Statistical analysis was a one-way ANOVA; **, P<0.01, ***; P<0.001 and ns, not significant. The depicted western blot is representative for all experiments. Expected positions of p53 and GAPDH are indicated. Numbers are indicating molecular weight in kDa of relevant protein ladder signals. (C) At indicated time points, taRNA-miR transfected HDFn cells were harvested to study TP53 gene expression by RT-qPCR. TP53 expression was normalized to that of HPRT and untreated cells (mean (SD) of n=3). Statistical analysis was a two-way ANOVA; **, P<0.01; ***, P<0.001; ****, P<0.0001; and ns, not significant.

FIG. 6: taRNA-miR-p53-2 is processed into mature miR-p53-2. HDFn cells were electroporated with the different taRNA-miR constructs (as indicated, either with active (VEE-repl) or inactive (GAA-) replicase, respectively; 0.787 pmol/RNA) or without RNA (mock). (A)₇₂ h after transfection cells were pelleted, lysed and prepared to calculate relative TP53 gene expression by RT-qPCR. TP53 mRNA expression was normalized to that of HPRT and taRNA-miR-neco transfected cells. (B) At the same time after transfection additional cell pellets were lysed and prepared for miRNA-specific, LNA-based detection of miR-p53-2 by RT-qPCR. PCR cycle numbers (Cq value, mean of triplicates) of miR-p53-2, SNORD48 (reference gene) and UniSp6 are shown. UniSp6 RNA is a synthetic control template that was added to each sample prior cDNA synthesis reaction to serve as an internal quality control. A synthetic RNA oligo (0.5 fmol) consisting of the miR-p53-2 sequence served as positive control for the miR-p53-2 assays. LNA, locked nucleic acid.

FIG. 7: taRNA-miR-VIPs suppress RSAD2 (viperin) expression which is induced by taRNA-miR transfection. HDF cells were electroporated with 2.5-3 μg taRNA (0.787 pmol/RNA) or without RNA (mock). 72 h after transfection cells were pelleted, lysed and prepared to analyze RSAD2 transcript levels via RT-qPCR. Expression levels were normalized to that of HPRT and mock cells (mean (SD) of n=2). Statistical analysis was a one-way ANOVA; *, P<0.05, **; P<0.01;

FIG. 8: Incorporating endogenous miR-302/367 cluster into taRNA-miR leads to downregulation of target genes. (A) Illustration of a taRNA-miR vector comprising the stem cell-specific miR-302/367 cluster. (B, C) HDFn cells were electroporated with the indicated taRNA-miR constructs (0.787 μmol/RNA) and 0.5 μg E3 mRNA and 0.5 μg B18R mRNA or left untreated. Lipofection with 0.4 μg of mature miR-302/367 composed of miR-302a, -302b, -302c, -302d and 367 (0.4 μM each) served as control. Endogenous TGFβR2 and DAZAP2 transcript levels were analyzed (B) 72 h and (C) 144 h after transfection by RT-qPCR. mRNA expression was normalized to that of HPRT and untreated cells (mean (SD) of n=3). Statistical analysis was a one-way ANOVA; *, P<0.05; **, P<0.01; ***, P<0.001; ****, P<0.0001 and ns, not significant.

FIG. 9: Incorporation of pre-miRNA into the 3′UTR of protein-coding transreplicons preserves high protein expression and enables target gene regulation. (A) Scheme of a taRNA-miR vector. taRNA comprises two capped and poly-adenylated RNA molecules, one non-replicative mRNA coding for the VEEV replicase (nrRNA-REPL) and a short transreplicon (STR-miR) that is replicated by the VEEV-replicase, and coding for a transgene (TG) alongside a miRNA upstream of its 3′ untranslated region (UTR) and conserved sequence element (CSE). STR-miR is recognized as primary miRNA by ribonuclease Drosha and processed into precursor miRNA (pre-miRNA), and a 5′- and a 3′-truncated STR-fragment. Pre-miRNA is further processed into miRNA duplex by Dicer, another ribonuclease. (B-E) BHK-lacZ cells were electroporated with the respective taRNA-miR constructs (2.2 μM per STR-miR and 1.6 μM nrRNA-REPL of TRD-VEEV), or without RNA (mock). (B) taRNA-miR GFP expression. 24 h after transfection the percentage of GFP-positive cells and GFP-mean fluorescence (MFI) were determined by flow cytometry (mean (SD) of n=3). (C) Target transcript level. Total cellular RNA was harvested 72 h after transfection to quantify relative transcript levels of lacZ normalized to that of β-actin by qRT-PCR. Mock-electroporated cells served to determine mean fold changes (mean (SD) of n=3). Statistical significances were tested by one-way ANOVA; **, P<0.01, and ns, not significant corresponding to mock. (D) Target gene expression. β-galactosidase (β-Gal) expression was measured at the indicated time points and normalized to that of mock-electroporated cells (mean (SD) of n=3). Statistical significances were tested by two-way ANOVA; **, P<0.01, and ns, not significant corresponding to mock (E) Cell viability. The viability of BHK-lacZ cells was determined at indicated time points and normalized to that of mock-electroporated cells (mean (SD) of n=3). Statistical significances were tested by two-way ANOVA; ns, not significant corresponding to mock. vUTR, viral UTR; nsP, non-structural protein; miR-neco; non-targeting miRNA control; miR-scrmbld, no miRNA processing control; TRD, Trinidad donkey strain.

FIG. 10: Replication of STR-miR is required for target knockdown and replicase activity determines the extent of knockdown. BHK-21 cells stably expressing firefly luciferase (BHK-luc) were electroporated with 1.1 μM of indicated STR-miR and co-delivered with 0.4 μM of either inactive replicase (inactive-REPL), replicase of VEEV-TRD (TRD-REPL) or hyperactive replicase (hyper-REPL). Control cells were electroporated without RNA (mock). (A) Target gene expression. Luciferase expression was measured at indicated time points and normalized to that of mock electroporated BHK-Luc cells (mean (SD) of n=3). Statistical significances were tested by two-way ANOVA; *, P<0.1; **, P<0.01; **, P<0.001; ***, P<0.0001, and ns, not significant corresponding to mock. (B) Cell viability and proliferation. Viability of BHK-luc cells was determined at indicated time points after transfection (mean (SD) of n=3). luc, luciferase; RLU, relative light units; VEEV, Venezuelan equine encephalitis virus; TRD, Trinidad donkey strain.

FIG. 11: taRNA-miR mediates sustained suppression of an endogenous target in primary cells and mature miRNAs accumulate during replication. (A-E) HDF cells were electroporated with the indicated taRNA-miR constructs (0.8 μM/RNA) and 0.2 μM E3 mRNA and 0.2 μM B18R nrRNA or without RNA (mock). As controls, 30 nM synthetic scrambled siRNA or synthetic siRNA against TP53 were transferred by lipofection. (A) Target transcript level. Total cellular RNA was harvested 72 h after transfection to quantify relative transcript levels of TP53 normalized to that of HPRT by qRT-PCR. taRNA-miR-neco-transfected cells served to determine mean fold changes. (B) Target protein expression. P53 protein levels were assessed 72 h after transfection by western blotting. The depicted blot is representative for three independent experiments. Expected molecular weights in kilodalton (kDa) of p53 and GAPDH are indicated. The bar chart summarized p53 expression levels quantified by densitometry, normalized to GAPDH expression per lane. The p53 protein level in taRNA-miR-neco transfected cells served to determine mean fold changes. For (A) and (B) mean (SD) of three independent experiments were calculated and statistical significances were tested by one-way ANOVA; *, P<0.05; **, P<0.01, *; P<0.001 and ns, not significant corresponding to taRNA-miR-neco. (C) TP53 transcript level over time. Total cellular RNA was harvested at indicated time points after transfection to quantify relative transcript levels of TP53 normalized to that of HPRT by qRT-PCR. Mock-electroporated cells served to determine mean fold changes. Statistical analysis was a two-way ANOVA; **, P<0.01; **, P<0.001; *, P<0.0001 and ns, not significant corresponding to mock (D) STR-miR RNA level over time. Total cellular RNA was harvested at indicated time points after transfection to quantify relative transcript levels of STR-miR normalized to that of HPRT by qRT-PCR. To detect STR-miR transcripts, primers specific for SecNluc (transgene) were used. (E) Mature miR-p53-2 levels. Total cellular RNA was harvested at indicated time points after transfection to quantify absolute levels of mature miR-p53-2 processed from STR-miR-p53-2 in cells co-transfected with inactive (inactive-REPL) or active replicase (TRD-REPL) determined by qRT-PCR. All graphs shown are the mean (SD) of three independent experiments.

FIG. 12: A polycistronic miRNA cluster is processed from taRNA-miR and targeted genes are suppressed for several days. (A) Illustration of STR-miR-302/367 vector. STR-miR-302/367 incorporates the natural human miR-302/367 cluster composed of the five miRNAs miR-302b, -c, -a, -d and -367. (B/C) HDF cells were electroporated with the indicated taRNA-miR constructs (0.8 μM/RNA) and 0.2 μM E3 and 0.2 μM B18R mRNA or without RNA (mock). Lipofection of HDF cells with synthetic mature miRNA miR-302a-d and 367 (0.4 UM each) served as positive control. (B) Relative miRNA levels. Total cellular RNA was harvested three and six days after transfection to quantify relative mature miRNA levels of miR-302s (miR-302a, miR-302b, miR-302c, and miR-302d) and miR-367 normalized to that of SNORD48 by qRT-PCR. taRNA-miR-neco-transfected cells served to determine mean fold changes (mean (SD) of n=3). (C) Target transcript levels. Total cellular RNA was harvested three and six days after transfection to quantify relative transcript levels of TGFβR2 and DAZAP2 normalized to that of HPRT by qRT-PCR. Mock-electroporated cells served to determine mean fold changes (mean (SD) of n=3). Statistical analysis was a one-way ANOVA; *, P<0.05; **, P<0.01; **, P<0.001; **, P<0.0001 and ns, not significant corresponding to mock. iPSCs, induced pluripotent stem cells.

FIG. 13: Replication steps of TR and STR RNA. In vitro-transcribed (IVT) non-replicative mRNA coding for the VEEV replicase (nrRNA-REPL) is translated into immature replicase (imm. REPL) and first processed into negative-strand-specific replicase complex ((−) REPL) and then further cleaved to eventually form fully mature positive-strand replicase complex ((+) REPL). The replication processes of the transreplicon (TR) (left) and short transreplicon (STR) (right) involve the synthesis of a negatively sensed copy of the positively sensed transfected IVT RNA vectors in full length, followed by transcription of novel full-length positive-sensed copies ((+) TR-TG or (+) STR-TG, respectively). These steps mirror the replication of genomic RNA of alphaviruses. For transgene-encoding TR (TR-TG), replication additionally results in the transcription of a subgenomic RNA (sgRNA-TG), which is translated into the transgene (left, dashed box no. 1). STR replication, however, no longer supports subgenomic transcription due to codon mutations, (right). Consequently, it is limited to genomic-like replication, and all positive-stranded copies are translated exclusively to the transgene (right, dashed box no. 2). Adapted from Perkovic et al. 27

FIG. 14: Stable overexpression of miR-lacZ leads to efficient knockdown of β-galactosidase in a stable reporter cell line. BHK-lacZ cells expressing β-galactosidase were transduced with a lentivirus containing the emGFP-pre-miR-neco or-lacZ expression cassette or left untreated. (A) Transduction efficiency. For each experiment, transduction rates were monitored 48 h after transduction by determining the percentage of GFP-positive cells using flow cytometry (mean (SD) of n=3). (B) Target gene expression. β-galactosidase expression was measured at indicated time points and normalized to that of untreated cells (mean (SD) of n=3). Statistical significances were tested by two-way ANOVA; ****, P<0.0001, and ns, not significant corresponding to mock. (C) Target transcript level. Total cellular RNA was harvested 96 h after transfection to quantify relative transcript levels of lacZ normalized to that of β-actin by qRT-PCR. Untreated cells served to determine mean fold changes (mean (SD) of n=3). Statistical significances were tested by one-way ANOVA; **, P<0.01; *, P<0.001; *, P<0.0001, and ns, not significant. β-Gal, β-galactosidase.

FIG. 15: Predicted secondary structures of pre-miR-lacZ. Predicted secondary structure of pre-miR-lacZ and pre-miR-scrambled using mFOLD version 2.3 (default settings applied and assessed in Feb. 21, 2023). The guide and passenger strands are highlighted in blue and grey, respectively. Drosha cleavage sites are market with black arrows. Dicer cleavage sites are marked with white arrows. The online software tool “Shuffle DNA” on bioinformatics.org (SMS version 2) was used to create a sequence scramble of the pre-miR-lacZ sequence.

FIG. 16: Enhanced STR-miR-mediated emGFP expression in cells co-transfected with hyperactive replicase. BHK-21 cells stably expressing firefly luciferase (BHK-luc) were electroporated with 1.1 μM of indicated STR-miR and co-delivered with 0.4 μM of either inactive replicase (inactive-REPL), wild-type replicase (TRD-REPL) or hyperactive replicase (hyper-REPL) or without RNA (mock). 24 h after transfection the rate of emGFP-positive cells and emGFP-mean fluorescence (MFI) were determined by flow cytometry. Total GFP expression was approximated by multiplying the rate of emGFP-positive cells with the MFI of emGFP-positive cells.

FIG. 17: Co-transfection of E3 and B18R nrRNA enhances transgene expression of taRNA and reduces toxicity in primary cells. Human foreskin fibroblasts were electroporated with 0.8 pM STR-luc or taRNA (including 0.8 μM STR-luc and 0.4 μM VEEV replicase) co-transfected with or without 0.3 μM E3 and 0.5 μM B18R nrRNA (EB). Control cells were electroporated without RNA (mock). (A) taRNA luc expression. Luciferase expression was measured at indicated time points after transfection (mean (SD) of n=3). (B) Cell Viability. Viability of cells was determined at indicated time points after transfection and normalized to that of mock cells (mean (SD) of n=3). RLU, relative light units. For (A) and (B) statistical significances were tested by two-way ANOVA; ****, P<0.0001 corresponding to taRNA.

FIG. 18: taRNA-miR-p53 constructs are non-toxic and not impaired in transgene expression. HDF cells were electroporated with the indicated taRNA or taRNA-miR constructs (0.8 μM/RNA) and 0.2 μM E3 and 0.2 pM B18R mRNA or without RNA (mock). (A) Cell viability. Viability of cells was determined at indicated time points after transfection and normalized to that of mock cells (mean (SD) of n=3). Statistical significances were tested by two-way ANOVA; ns, not significant corresponding to mock. (B) taRNA GFP expression. The percentage of GFP-positive cells and the GFP-mean fluorescence (MFI) were determined 24 h after transfection by flow cytometry. The bar charts summarize three independent experiments (mean (SD)). The dot plots show cell distribution and GFP expression of a representative experiment. Side versus forward scatter (FSC and SSC) was used to gate on intact HDF cells (first and third row). GFP-fluorescence of gated cells was plotted against an irrelevant channel (APC-Cy7, second and fourth row). MFI, mean fluorescence intensity.

### EXAMPLES I

### Materials and Methods

### Cell Culture

Unless indicated otherwise, all chemicals were supplied by Life Technologies/Gibco. BHK-21 cells (ATCC; CCL-10) and derived transductants were grown in Eagle's minimal essential medium supplemented with 10% FCS. HDF cells (ATCC PCS-201-010) were grown in supplemented fibroblast medium (Fibroblast Medium Kit from Innoprot). All cells were cultivated at 37 °C in humidified atmosphere equilibrated to 5% CO2.

Alphaviral Vectors and miRNA

For in vitro synthesis of miRNA-encoding taRNA (taRNA-miR), the coding sequence of the Trinidad donkey (TRD) Venezuelan Equine Encephalitis virus replicase (VEEV active replicase, accession number L⁰¹⁴⁴², inactive VEEV replicase, nsP4^{GDD->GAA}, with a GDD-to-GAA mutation in the active site of nsP4 polymerase as described in Gotte et al., 2020, J Virol, 94: e01681-19) was inserted into Spel and NotI sites of the latest generation of the pST1 vector backbone (Beissert et al., 2020, Mol Ther 28 (1): 119-128; Stadler et al., 2017, Nat Med 23, 815-817; Orlandini et al., 2019, Mol Ther 27 (4): 824-836.; Kuhn et al., 2010, Gene Ther 17, 961-971; Holtkamp et al., 2006, Blood 108 (13): 4009-4017).

The NTRs were generated similarly to what was previously described (WO 2017/162460 A1, Beissert et al., 2020, Mol Ther 28 (1): 119-128). Two lentiviral vectors containing emGFP-pre-miRNA expression cassettes (BLOCK-IT™ Lentiviral Pol II miR RNAi Expression System with EmGFP Kit, Catalog no. K4925-00, Invitrogen) were used as PCR templates to clone the miRNA cassettes into NTR vectors. The miRNA vectors include flanking and loop sequences from an endogenous miRNA (murine miR-155 sequence, Lagos-Quintana et al., 2002, Curr Biol 12 (9): 735-739), which directs the excision of the engineered miRNA from a longer Pol II transcript (pri-miRNA). One of the supplied plasmids contains an engineered pre-miRNA sequence that is directed against lacZ (named miR-lacZ), whereas the negative control plasmid comprises a pre-miRNA sequences that forms a hairpin structure that is processed into mature miRNA but forecasted to not target any known vertebrate gene (named miR-neco). Additionally, a scrambled miR-lacZ control was included. For this an online software tool on bioinformatics.org was used to create a sequence scramble of the pre-miR-LacZ sequence. All other miRNA sequences were designed using the BLOCK-IT RNAi designer (https://rnaidesigner.thermofisher.com/rnaiexpress/; for miRNA sequence details see Table 1). In silico designed taRNA-miR constructs were synthetized by the biotech company GENEWIZ. Delivered gene fragments were PCR-amplified and poly(A)-tailed with a specific antisense primer (all PCR primer sequences designed and used are listed in Table 2).

TABLE 1
Pre-miRNA sequences.

| Name | DNA sequence from 5′ to 3′ |
|---|---|
| Pre-miR-lacZ | TGCTGAAATCGCTGATTTGTGTAGTCGTTTTGGCCACTGACTGACGACTACACATCAGCGATTT |
| (SEQ ID NO: | |
| 2) | |
| Pre-miR- | TGCTGAAATGTACTGCGCGTGGAGACGTTTTGGCCACTGACTGACGTCTCCACGCAGTACATTT |
| neco (SEQ ID | |
| NO: 3) | |
| Pre-miR-luc1 | TGCTGAGCCCATATCGTTTCATAGCTGTTTTGGCCACTGACTGACAGCTATGACGATATGGGCT |
| (SEQ ID NO: | |
| 4) | |
| Pre-miR-luc2 | TGCTGATACCTGGCAGATGGAACCTCGTTTTGGCCACTGACTGACGAGGTTCCCTGCCAGGTAT |
| (SEQ ID NO: | |
| 5) | |
| Pre-miR- | TGCTGTCCACACGCAAATTTCCTTCCGTTTTGGCCACTGACGGAAGGAATTGCGTGTGGA |
| p53-1 (SEQ | |
| ID NO: 6) | |
| Pre-miR- | TGCTGAGTAGATTACCACTGGAGTCTGTTTTGGCCACTGACAGACTCCAGGTAATCTACT |
| p53-2 (SEQ | |
| ID NO: 7) | |
| Pre-miR- | TGCTGCAAACACGCACCTCAAAGCTGGTTTTGGCCACTGACCAGCTTTGGTGCGTGTTTG |
| p53-3 (SEQ | |
| ID NO: 8) | |
| *Scrambled | ATGGATCCCACCAAGTTGAGTTACGTACTAAGTTCTGAGTCGTCGCCCGCGTTTATTGATATGT |
| control (SEQ | |
| ID NO: 9) | |
| Pre-miR- | TGCTGTCTATAATCCCTACACCACCTGTTTTGGCCACTGACTGACAGGTGGTGGGGATTATAGA |
| VIP1 (SEQ | |
| ID NO: 10) | |
| Pre-miR- | TGCTGTACCAATCCAGCTTCAGATCAGTTTTGGCCACTGACTGACTGATCTGACTGGATTGGTA |
| VIP2 (SEQ | |
| ID NO: 11) | |

Mature miRNA sequences are in bold;
*randomly shuffled pre-miR-lacZ sequence

TABLE 2
PCR primers
(homology overhangs for cold fusion reaction are indicated in bold letters).

| Primer name | Sequence |
|---|---|
| VEEV-5CSE- | CCGCAGAAAGTATTCTACTAGTCCACCATGGTGAGCAAGGGC |
| emGFP-fw (SEQ | |
| ID NO: 12) | |
| VEEV-SGP- | ATAGTCTAGTCCGCCAAGACTAGTCCACCATGGTGAGCAAGGGC |
| emGFP-fw (SEQ | |
| ID NO: 13) | |
| miR-3CSE-VEEV- | AATTGCTGCTGTATTCATGCGGCCGCTGGGCCATTTGTTCCATGTG |
| rv (SEQ ID NO: | |
| 14) | |
| miR-scrambled- | TATCAATAAACGCGGGCGACGACTCAGAACTTAGTACGTAACTCAACTTGGTGGGATCCATTACA |
| lacZ-rv1 (SEQ ID | GCCTTCAGCAAGCC |
| NO: 15) | |
| miR-scrambled- | AATTGCTGCTGTATTCATGCGGCCGCTGGGCCATTTGTTCCATGTGAGTGCTAGTAACAGGCCTT |
| VEEV-rv2 (SEQ | GTGTCCTGACATATCAATAAACGCGGGCGAC |
| ID NO: 16) | |
| SINV-SGP- | ACTACAACACCACCACCCGATCGCCACCATGGTGAGCAAGGGC |
| emGFP-fw (SEQ | |
| ID NO: 17) | |
| SINV-3CSE- | GCTCGTAGAACGATAAACCCGGGCCACCATGGTGAGCAAGGGC |
| emGFP-fw (SEQ | |
| ID NO: 18) | |
| miR-3CSE-SINV- | GCAGACGCTGCGTGGCGGCCGCGATCTGGGCCATTTGTTCCATGTG |
| rv (SEQ ID NO: | |
| 19) | |
| miR-scrambled- | GCAGACGCTGCGTGGCGGCCGCGATCTGGGCCATTTGTTCCATGTGAGTGCTAGTAACAG |
| SINV-rv2 (SEQ | GCCTTGTGTCCTGACATATCAATAAACGCGGGCGAC |
| ID NO: 20) | |
| VEE-pA60-rv | TTTTTTTTTTTTTTTTTTTTTTTTTTTTTTTTTTTTTTTTTTTTTTTTTTTGAAATATTA |
| (SEQ ID NO: 21) | AAAACAAAATCCGATTCGGAAAAG |
| VEE-T7-5UTR- | TAATTAATACGACTCACTATAGATGGG |
| ATGG (SEQ ID | |
| NO: 22) | |

### In Vitro RNA Transcription

For in vitro transcription, all template plasmids were linearized downstream of the poly-(A) tail by digestion with the restriction enzyme BspQI. Linearized plasmids were then purified using Dynabeads MyOne Carboxylic Acid (Invitrogen, Waltham, MA, USA) according to the manufacturer's instructions. Synthesis and purification of RNA were previously described (Kuhn et al., 2010, Gene Ther 17, 961-971; Holtkamp et al., 2006, Blood 108 (13): 4009-4017). Quality of purified RNA was assessed by spectrophotometry, and analysis on the 5200 Fragment Analyzer (Advanced Analytical).

### Production of Viral Supernatants and Lentiviral Transduction of Cells

Lentiviral particles were generated by co-transfection of HEK293T-17 cells with the respective lentiviral vector together with the GAG-POL expressing packaging plasmid pCMVAR8.91 and the VSV-G envelope plasmid MD2G using TransitLT1 (Mirus Bio, Madison, WI, USA). The respective crude lentiviral supernatants were harvested and loaded three times onto non-tissue culture treated plates coated with 20 mg/mL Retronectin (Takahara, Clonetech Laboratries, USA) by centrifugation (1.500×g, 15 min, 15 °C). After washing with PBS to remove unbound virus particles, BHK-21 cells were plated and incubated overnight at 37 °C for transduction. Infected bulk populations expressing Fluc/lacZ/miR-lacZ/miR-neco were used for the experiments.

TABLE 3
2^{nd} Generation lentiviral plasmids used for the production of viral supernatants

| Envelope plasmid | Packaging plasmid | Transfer plasmid | Generated |
|---|---|---|---|
| -constant- | -constant- | -varies- | transductants |
| *VSV-G (pantropic | Gag (matrix, capsid and | pLenti6.4/CMV-V5-MSGW/lacZ | BHK-lacZ cells |
| envelope protein) | nucleocapsid), Pol | bacterial/acZ gene | |
| | (reverse transcriptase | | |
| | and integrase), Rev | pL64E-(EF1a-emGFP- | BHK-lacZ-emGFP- |
| | (facilitates nuclear | miRNA-LacZ)-Blast | miR-lacZ cells |
| | export) and Tat (trans- | encoding emGFP-miR-lacZ | |
| | activator) | | |
| vector pMD2G | vector pCMVAR8.91 | pL64B42E-(EF1a-emGFP- | BHK-lacZ-emGFP- |
| | | miRNA_negctr)-Blast | miR-neco cells |
| | | encoding emGFP-miR-neg-ctrl. | |

*VSV-G: Vesicular stomatitis virus G glycoprotein.

Total RNA Extraction and RT-qPCR to Analyze mRNA Expression

To obtain purified total RNA from cell lysates, the RNeasy Mini Kit (Qiagen, Hilden, Germany) was used according to the manufacturer's instructions. The purity and yield of total RNA were assessed by 260/280 and 260/230 absorbance ratio, using a NanoDrop 2000 UV-Vis spectrophotometer (Thermo Fisher Scientific, Waltham, MA, USA). For qPCR analysis of mRNA transcripts, total RNA was used for reverse transcription with oligo (dT)₁₈ primer and the Superscript IV Reverse Transcriptase (Invitrogen, Carlsbad, USA). The cDNA products were 1:10 diluted to serve as templates for qPCR. SYBR green-based quantitative real-time PCR analysis was performed. Protocol followed the manufacturer's instruction with 15 min at 95 °C, and 40 cycles of 30 sec at 95 °C, 30 sec at 60 °C, and 30 sec at 72 °C. Analyses were performed using the 2-AACT method (Livak and Schmittgen. 2001, Methods 25 (4): 402-408), normalized to the reference gene HPRT (HDF cells) or β-actin (BHK-21 cells). The sequences of the qPCR primers are listed in Table 4.

TABLE 4
Primer pairs designed and used for qPCR reactions.

| | primer sequence | |
|---|---|---|
| Target | sense | antisense |
| Human HPRT (SEQ | TGACACTGGCAAAACAATGCA | GGTCCTTTTCACCAGCAAGCT |
| ID NO: 23 & SEQ ID | | |
| NO: 24) | | |
| *Hamster β-actin | CCTGTATGCCAACACAGTGC | ATACTCCTGCTTGCTGATCC |
| (SEQ ID NO: 25 & | | |
| SEQ ID NO: 26) | | |
| Bacterial lacZ gene | GTACGTCTTCCCGAGCGAAA | CTGTTGACTGTAGCGGCTGA |
| (SEQ ID NO: 27 & | | |
| SEQ ID NO: 28) | | |
| Human p53 gene | ACACTCGCTTCTGAATCATC | GAGACCATTCATAAGCAACG |
| (SEQ ID NO: 29 & | | |
| SEQ ID NO: 30) | | |
| Human RSAD2 gene | TGCCACAATGTGGGTGCTTACAC | CTCAAGGGGCAGCACAAAGG |
| (SEQ ID NO: 31 & | | |
| SEQ ID NO: 32) | | |
| Human TGFβR2 | TGAGTCCTTCAAGCAGACCGA | ACACACCATCTGGATGCCCTG |
| (SEQ ID NO: 33 & | | |
| SEQ ID NO: 34) | | |
| Human DAZAP2 | CGAACAGGAAGAGGACGAAA | CAGGGTAGGTTGGCTGTGTT |
| (SEQ ID NO: 35 & | | |
| SEQ ID NO: 36) | | |

*Hamster: Species Mesocricetus auratus and therefore suitable for BHK21 cell line

Total RNA Extraction and RT-qPCR to Analyze miRNA Expression

To obtain small RNA-containing total RNA from cell lysates, the mirVana miRNA Isolation Kit (Ambion-1561, Austin TX, USA) was used according to the manufacturer's instructions. The purity and yield of the total RNA were assessed by 260/280 and 260/230 absorbance ratio, using a NanoDrop 2000 UV-Vis spectrophotometer (Thermo Fisher Scientific, Waltham, MA, USA). cDNA was made using miRCURY LNA RT Kit (Qiagen, Hilden, Germany) based on the Poly(A) reverse-transcription PCR method. 20 ng RNA was used according to the manuals for each 20 μl reverse-transcription reaction. The cDNA synthesis quality was controlled with the use of UniSp6 target, which is provided in the cDNA synthesis kit. 1 μl of UniSp6 RNA was added to each sample prior cDNA synthesis reaction. The miRCURY LNA miRNA custom PCR Assay was used for the detection of mature miR-p53-2 in transfected cells. Specific forward and reverse primers were designed with the Qiagen online software. A synthetic RNA oligo (0.5 fmol) consisting of the miR-p53-2 sequence served as positive control for the miR-p53-2 assays. Specific primers were used for SNORD48, which served as reference gene. Relevant primers provided in the kit were used to quantify the synthetic control template UniSp6. cDNA was diluted 60× with nuclease free water, and a mixture of 5 μL miRCURY LNA SYBR Green, 1 μL miRCURY LNA miRNA Assay (primers), 3 UL diluted cDNA, and 1 μL nuclease-free water per reaction was prepared. qPCR was performed with the use of a Bio-Rad C₁₀₀₀ Touch Thermal Cycler, according to the miRCURY LNA miRNA SYBR Green PCR handbook and the given qPCR program (October 2019). Calculated Cq values (mean of triplicates) and melt curves for each target were obtained from instrument software.

### RNA Transfection

RNA was electroporated into cells (0.5-1E+06 cells/electroporation) at room temperature by applying defined pulses with a square wave electroporator (BTX ECM 830, Harvard Apparatus, Holliston, MA, USA). For each electroporation of parental BHK-21 cells or the derived transductants, one electric pulse of 750 V/cm of 16 milliseconds (ms) was applied. HDF cells were electroporated with three electric pulses of 625 V/cm of 12 ms. siRNA lipofections (p53 siRNA sc-29435 and siRNA-A (scrambled) sc-3700, Santa Cruz Biotechnology, Inc., Heidelberg, Germany) were performed using Lipofectamine RNAiMAX following the manufacturer's instructions (Life Technologies, Darmstadt, Germany). For all experiments, molarities or amounts of RNAs were used as indicated in the figure legends. After transfection, cells were incubated without refreshing medium until analysis.

### Luciferase-Based Assays

### Bright-Glo® Luciferase Assay

The luciferase-based reporter assay is a rapid and reliable tool for assaying gene expression of the reporter gene luciferase (Nguyen et al., 1988, O. Anal Biochem 171 (2): 404-408). The firefly luciferase (Photinus pyralis) is immediately functional after translation, catalysing the mono-oxygenation of beetle luciferin under light emission (550-570 nm). The bioluminescent reporter was measure by plating cells stably expressing luciferase, in triplicates, on black 96-well plates. Per well, 5E+03 cells, in a total volume of 50 μL, were prepared. In 24 h intervals, the luciferase expression was measured using the Bright-Glo® Luciferase Assay system (Promega, Madison, WI, USA). Therefore, 30 μL of reconstituted reagent (containing a lysis buffer) were added to each well, mixed and incubated for three min at room temperature, while protected from light. Bioluminescence (photons per second) was measured using a microplate reader Infinite M200 (Tecan Group, Mannedorf, Switzeland).

### BetaGlo® Assay System

To quantify the expression of ß-galactosidase proteins, a coupled-enzyme reaction of the Beta-Glo® Assay system (Promega, Madison, WI, USA) was used. In this particular assay, the light reaction depends on the amount of β-galactosidase present in a sample. The added substrate, 6-O-ß-galactopyranosyl-luciferin, is cleaved by β-galactosidase generating luciferin, which is further catalysed by luciferase present in the BetaGlo® reagent. Cells, stably expressing β-galactosidase, were plated in quintuplicates on black 96-well plates to a number of 5E+03 cells per well in a total volume of 50 μL. Again, in 24 h intervals, 50 μL BetaGlo® reagent was added to the cells, mixed and incubated for one hour at room temperature before the measurement.

### CellTiterGlo® Luminescent Cell Viability Assay

Relative cell vitality was determined using CellTiterGlo® Luminescent Cell Viability Assay (Promega, Madison, WI, USA). The CellTiterGlo® reagent comprises a thermostable luciferase (Ultra-Glo™ recombinant luciferase), the substrate luciferin and cofactors except of ATP. Hence, the amount of luminescence generated directly correlates with the amount of ATP present in a sample. Subsequently, the quantity of ATP is an indicator of metabolically active cells. For the determination of the cytotoxic effects of RNA replication in respective transfection experiments, cells were plated and bioluminescence was measured as described in “Bright-Glo® Luciferase Assay”, above, with the alteration that 50 μL of the respective reagent was used instead of 30 μL. The amount of viable cells was calculated referred to the negative control of cells, untreated or transfected without RNA.

### Flow Cytometric Analysis

For flow cytometric analysis of fluorescent protein expression, the cells were harvested, washed once with PBS, and fixed with PBS containing 4% formaldehyde. Expression of fluorescent proteins was assessed using FACS Canto II flow cytometer (BD Bioscience, Heidelberg, Germany) and the companion Diva software. FlowJo software was used for further data analyses.

### Western Blots

Total cell extracts were generated by solving the cell pellets in RIPA buffer and 100× Halt™ Protease and Phosphatase Inhibitor Cocktail (Thermo Scientific). Samples were incubated for 30 min at 4 °C on a rotatory-wheel followed by a centrifugation step (16,200×g, 4 °C for 15 min) to remove cell debris. Protein concentration in cell extracts (supernatant) was measured by Pierce BCA protein assay (Thermo Fisher Scientific, Waltham, MA, USA). Equal amounts of protein were loaded onto SDS-PAGE gels and protein transfer on nitrocellulose membrane (GE Healthcare, Marlborough, MA, USA) was performed by semi-dry western blot. Non-specific binding to the membranes was blocked with 1% (w/v) skim milk powder solutions in 1×PBS-Tween. Immunostaining with primary antibodies against p53 (Santa Cruz, sc-126) and GAPDH (GeneTex, GTX627408) was performed over night at 4 °C followed by secondary antibodies. Protein detection by chemiluminescence was performed using the Lumi-Light Western Blotting substrate (Roche, Basel, Switzerland) or Dura reagent (Thermo Fisher Scientific), and the ImageQuant LAS 4000 detection system (GE Healthcare). Quantification of signal intensity was performed using ImageQuant TL software (GE Healthcare). To compensate for unequal sample loading, relative expression values of proteins were normalized to corresponding relative signal intensities of loading controls. Normalized expression values were illustrated in bar blots.

### Statistical Analysis

Statistical analysis of data was performed by the own-way or two-way ANOVA with GraphPad Prism 9. Differences were considered significant if P-value was <0.05.

### EXAMPLE 1. VECTOR DESIGN AND MIRNA STRUCTURE

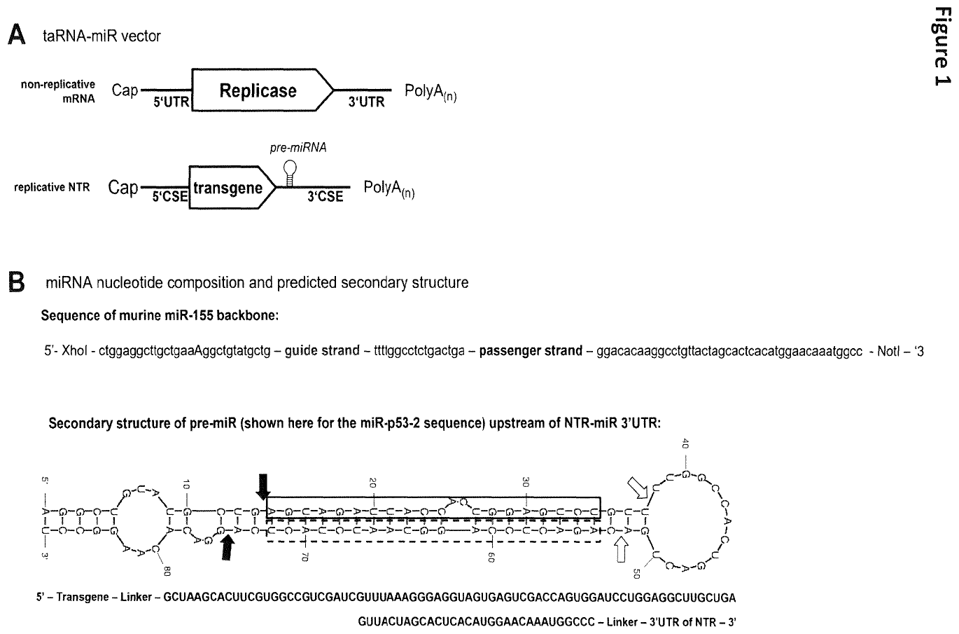

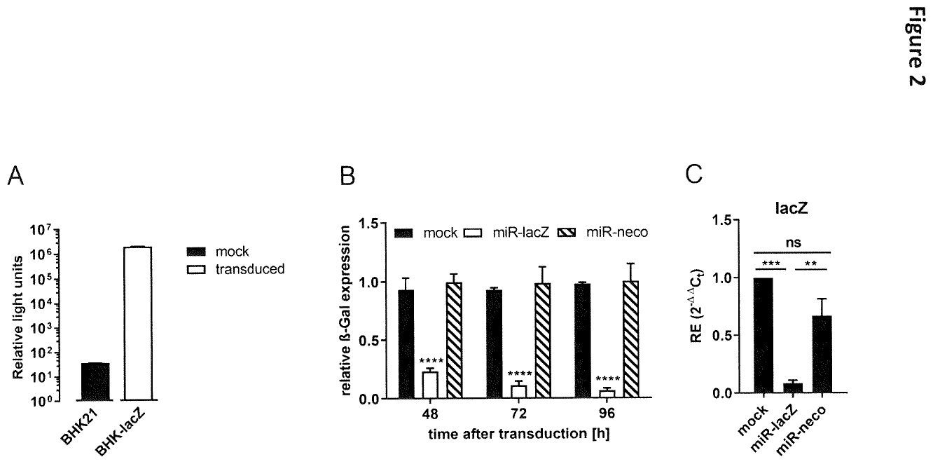

Trans-amplifying RNA systems (taRNA) were constructed from alphaviral genomes of the Venezuelan Equine encephalitis virus (VEEV), comprising a non-replicating mRNA encoding the VEEV-replicase and a nano-trans-replicon (NTR) encoding a transgene and a microRNA gene (FIG. 1A). The miRNA gene includes flanking and loop sequences from the murine miRNA-155 gene (Lagos-Quintana et al., 2002, Curr Biol 12 (9): 735-739), which direct the excision of the engineered miRNA (precursor-miRNA or pre-miR) from a longer Pol II transcript (primary-miRNA or pri-miR, FIG. 1B). Here, the pre-miR transcript is incorporated into the 3′UTR of the NTR (NTR-miR) resulting together with replicase mRNA in taRNA-miR system (FIG. 1A). For a proof of concept study, the miRNA gene was excised from a commercially available vector “BLOCK-iT™ Lentiviral Pol II miR RNAi Expression System with EmGFP” (Catalog no. K493800, Invitrogen, MA, USA) and inserted into the NTR. The pre-miRNA sequences found on the lentiviral plasmids were subcloned into taRNA vectors.

### EXAMPLE 2. STABLE OVEREXPRESSION OF MIR-LACZ LEADS TO EFFICIENT KNOCKDOWN OF β-GALACTOSIDASE

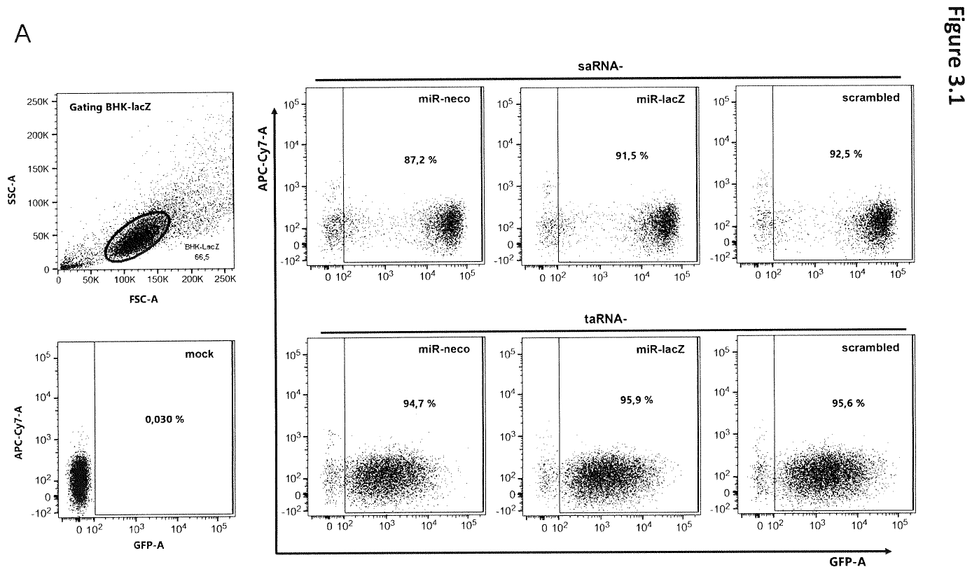

To assess the potential knockdown ability of the taRNA-miR system we first transduced baby hamster kidney 21 (BHK21) cells with a lentiviral vector for stable expression of β-galactosidase (β-gal) encoded by the lacZ gene. The transduced cell line was named BHK-LacZ (FIG. 2A). Next, a miRNA against the lacZ gene (miR-lacZ) or a negative control (miR-neco) were transferred into these cells using a commercial miRNA-expressing lentiviral vectors system, which enables high and persistent miRNA expression downstream of the emerald green fluorescence protein (emGFP). The successful transduction of the cells was confirmed by measuring emGFP expression, controls were left untransduced. miR-lacZ transduction with lentiviral vectors led to downregulation of β-gal (FIG. 2B) and the underlying lacZ RNA (FIG. 2C). LacZRNA level or β-gal expression of miR-neco transduced cells were not affected.

### EXAMPLE 3.1. INSERTING MIRNA INTO REPLICATING RNA DOES NOT AFFECT PROTEIN EXPRESSION IN CELLS

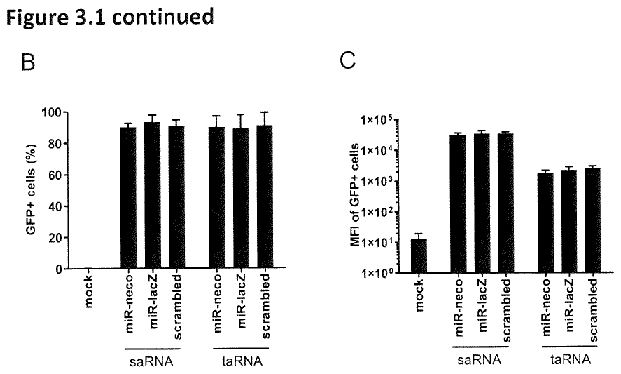

Self-amplifying RNA (saRNA) and taRNA comprising the reporter emGFP and a functional pre-miR sequence (pre-miR-lacZ or pre-miR-neco) were engineered to study microRNA delivery with replicative RNA. Excising a functional pre-miRNA transcript from saRNA or NTR would potentially release two products, the pre-miRNA transcript and a truncated saRNA or NTR RNA. Subsequently, the truncated RNA could no longer be replicated nor translated into protein. To control whether miRNA processing reduces RNA replication efficiency, the pre-miR-lacZ nucleotide sequence was scrambled to conserve length and nucleotide composition, but to disrupt the miRNA secondary structure. A disrupted pri-miR structure can no longer be recognized by drosha preventing pre-miRNA excision. To analyze if miRNA incorporation affects RNA replication and subsequently protein translation, the expression of the reporter emGFP was investigated by FACS. All transfected cells exhibited similar transfection rates and protein expression intensities (FIG. 3.1A-C). The insertion of a pre-miR transcript within the 3′UTR of alphaviral vectors did not have an impact on protein expression.

### EXAMPLE 3.2. ALPHAVIRAL MIRNA DELIVERY LEADS TO KNOCKDOWN OF LACZ

To test whether saRNA-/taRNA-miR-lacZ vectors can induce target downregulation, BHK-lacZ cells were transfected with the respective constructs and ß-gal expression was followed for 96 h. After 24 h, saRNA-miR-lacZ transfected cells showed significantly lower ß-gal expression levels compared to the controls (FIG. 3.2B). Strongest knockdown of β-gal was induced 72 h after transfection with a knockdown efficiency of 60% (FIG. 3.2B). taRNA-miR-lacZ transfection led to 50% downregulation of β-gal 72 h after transfection (FIG. 3.2E). On the RNA level, both saRNA-miR-lacZ and taRNA-miR-lacZ could reduce lacZ transcript levels by 70% relative to mock transfected cells (FIG. 3.2C,F). As expected, saRNA-miR transfections caused cytotoxicity with up to 70% viability loss (FIG. 3.2A). On the contrary, taRNA-miR transfections had only a minimal impact on cell viability (FIG. 3.2D). Alphaviral miRNA delivery with taRNA-miR leads to 70% target RNA knockdown and 50% reduced protein expression. While saRNA-miR transfection impairs viability, taRNA-miR does not.

### EXAMPLE 4. TARNA-MIR-LUC IS NON-CYTOTOXIC. IT DOWNREGULATES LUCIFERASE EXPRESSION ONLY WHEN ACTIVELY REPLICATED

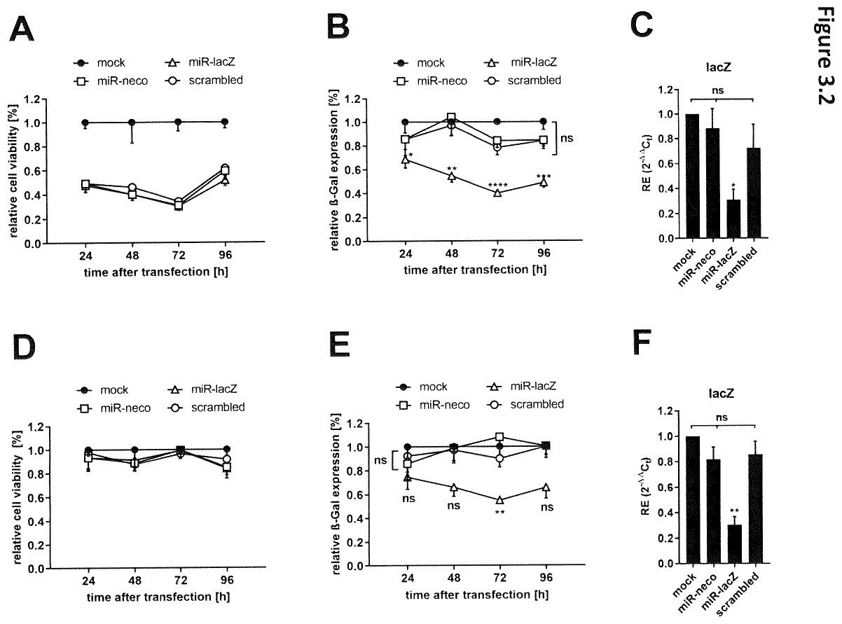

To confirm that taRNA-miR can significantly downregulate target mRNA, BHK21 cells were stably transduced with a lentivirus encoding the firefly luciferase gene (BHK-luc) (FIG. 4A). Two miRNAs were designed targeting firefly luciferase mRNA (miR-luc1 and miR-luc2) and cloned into the 3′UTR of NTR-emGFP. BHK-luc cells were transfected with the respective taRNA-miR vectors, and as a control with the NTR-miRs without co-delivering VEEV replicase mRNA. The downregulation of luciferase expression was assessed for 96 h after transfection. Without replicase mRNA, a trend to slightly but not significantly reduced luciferase expression was recognizable in NTR-miR-luc1 transfected cells (FIG. 4C). In contrast, both taRNA-miR-luc vector systems (i.e. +replicase) reduced luciferase expression significantly to 50-60% (FIG. 4E). As before, we observed no cytotoxicity induced by taRNA-miR or NTR-miR alone (FIG. 4B, D). In sum, this experiment confirmed that taRNA-miR is able to knockdown target genes without being cytotoxic. Furthermore, replication of the NTR-miR is a prerequisite for taRNA-miR-induced target downregulation.

### EXAMPLE 5. TARNA-MIR AGAINST TP53 DOWNREGULATES ENDOGENOUS P53

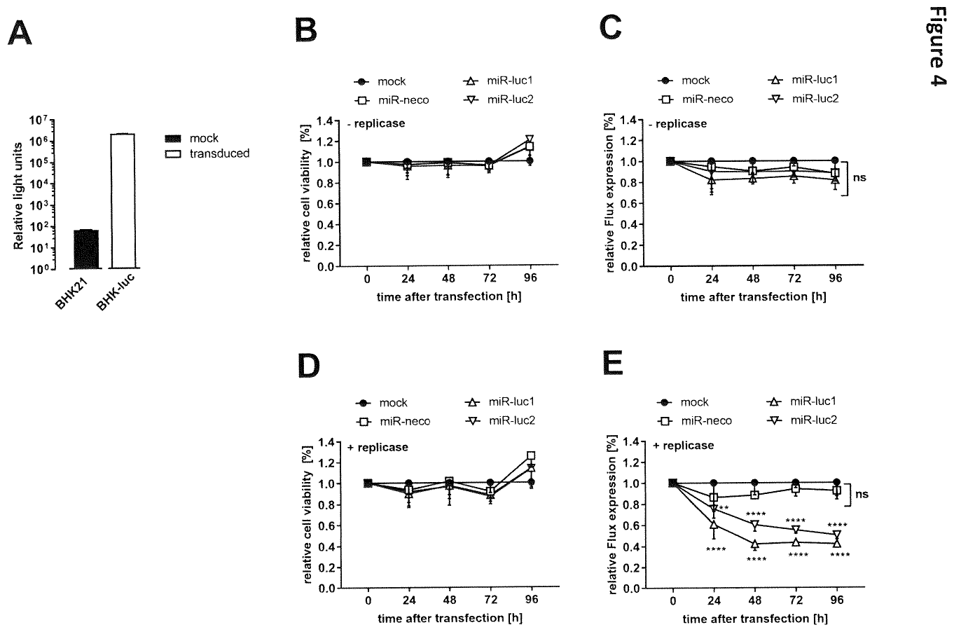

To demonstrate the downregulation of an endogenous transcript, three taRNA-miR constructs were designed comprising miRNA sequences against human TP53. Primary human fibroblasts were transfected with the respective constructs and TP53 RNA levels and p53 protein expression levels were examined 72 h post-transfection. P53 siRNA-transfected cells served here as a knockdown control. All taRNA-miR-p53 constructs were able to significantly downregulate endogenous p53 levels compared to taRNA-miR-neco or mock cells (FIG. 5A, B). Since taRNA-miR-p53-2 transfected cells showed the strongest p53 knockdown (~80%), TP53 expression in taRNA-miR-p53-2 transfected cells was monitored over time. To determine the kinetics of target downregulation in taRNA-miR-p53-2 transfected cells, TP53 levels were examined from 4 h to up to 96 h post-transfection. Significant differences in TP53 levels between taRNA-miR-p53-2 and controls where first observed 12 h after transfection. The maximum of ~80% TP53 knockdown was reached 24 h after transfection, remaining at this level for the next 72 h (FIG. 5C).

### EXAMPLE 6: TARNA-MIR-P53-2 IS PROCESSED INTO MATURE MIR-P53-2

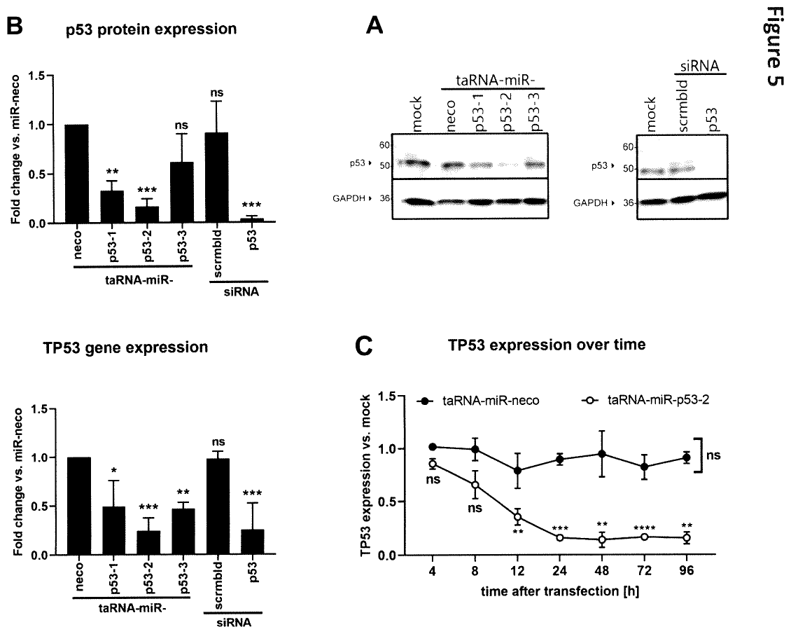

Repeatedly detected target gene knockdown shows that mature miRNAs were released from NTR-miR. We established qRT-PCR protocols detecting specifically mature miRNAs, and examined the levels of mature miR-p53-2 upon taRNA-miR transfer. The qPCR primers used specifically detect miR-p53-2, but not p53-1 or-3. Primary human fibroblasts were transfected with taRNA-miR-p53-1, -2 and -3 vectors or taRNA-miR-neco. To investigate the impact of NTR-miR replication on the level of mature miRNA, we included taRNA-miR-p53-2 comprising inactive replicase (GDD to GAA mutation in the catalytic site of nsP4) instead of active replicase. Total RNA was extracted 72 h after transfection. TP53 knockdown was achieved with all three taRNA-miR-p53, with p53-2 being the most effective as before. taRNA-miR-p53-2 made with inactive replicase was unable to regulate TP53 (FIG. 6A), although mature miR-p53-2 was detectable. However, in presence of the active replicase the level of mature miR-p53-2 was much greater. This proves that NTR-miR replication is required to achieve an effective miRNA level in the cells, leading to miR-specific target suppression (FIG. 6B).

### EXAMPLE 7: TARNA-MIR-VIPS SUPPRESS RSAD2 (VIPERIN) EXPRESSION WHICH IS INDUCED BY TARNA-MIR TRANSFECTION

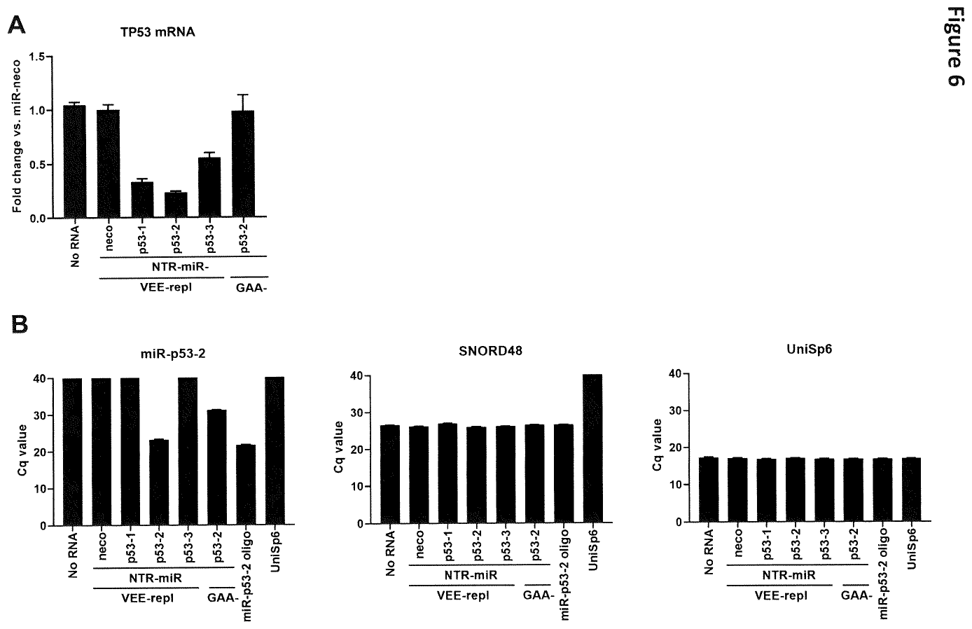

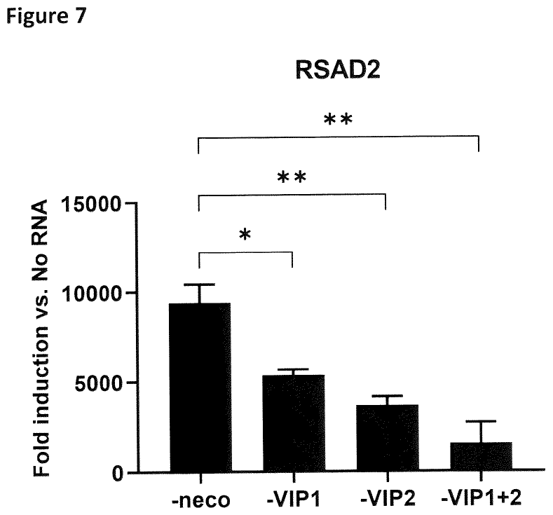

RSAD2 is an interferon stimulated gene (ISG), also known as viperin (VIP), that is highly upregulated upon alphaviral infection. Primary human fibroblasts were transfected with two different taRNA-miR-VIP constructs targeting RSAD2/viperin. taRNA-miR-neco transfected cells served again as control inducing a about 10,000-fold upregulation of RSAD2 transcript levels compared to mock cells. taRNA-miR-VIP1 or -VIP2 transfected cells showed 40-60% reduced RSAD2 levels compared to taRNA-miR-neco. Combining NTR-miR-VIP1 and -VIP2 with replicase mRNA reduced RSAD2 transcript levels even further (>80% 72 h after transfection) showing a synergistic effect (FIG. 7).

### EXAMPLE 8: INCORPORATING ENDOGENOUS MIR-302/367 CLUSTER INTO TARNA-MIR LEADS TO DOWNREGULATION OF TARGET GENES

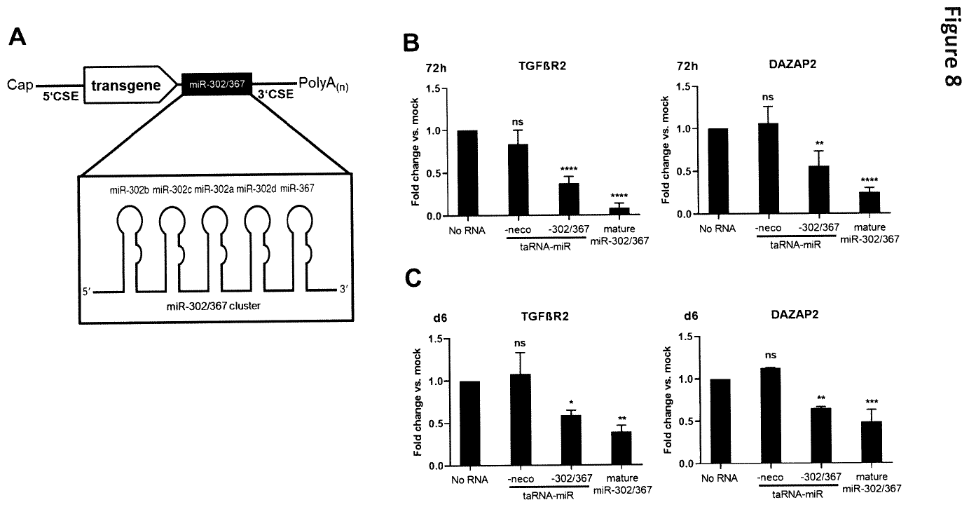

Next, we showed that the taRNA-miR vector system can be used to deliver a natural miRNA cluster. The endogenous miR-302/367 cluster, expressed in embryonic stem cells and induced pluripotent stem cells, is composed of five miRNAs, namely miR-302a-d and -367. We inserted the whole cluster sequence into the taRNA vector comparable to the synthetic miR-155 backbone before (FIG. 8A). This cluster regulates a plethora of genes and is involved in cell signaling, cell cycle, epigenetic regulation and glucose metabolism, among others. Two known targets are the genes DAZAP2 and TGFβR2. Human primary fibroblasts were transfected with taRNA-miR-302/367 and miR-neco, respectively or with mature miRNAs, which served as a positive control. 72 h after transfection, expression levels of both targets were significantly reduced in taRNA-miR-302/367 transfected cells compared to controls (FIG. 8B). Target gene suppression at a later time point was still visible and comparable in samples (FIG. 8C).

### EXAMPLES II

### Materials and Methods

Plasmids and RNA. Plasmids serving as templates for in vitro transcription of mRNA encoding the VEEV replicase (accession number L01442), an inactive replicase variant (as described in⁴⁹), an hyperactive replicase variant (as described in³³) and latest generation of TR (shortened TR, STR) were generated similarly to what was previously described.^{25; 27} Two lentiviral vectors containing emGFP-pre-miRNA expression cassettes were purchased (BLOCK-IT™ Lentiviral Pol II miR RNAi Expression System with emGFP Kit, Catalog no. K4925-00, Invitrogen) and used as PCR templates to clone the miRNA cassettes into STR vectors. The mature miRNA sequence targeting either bacterial lacZ gene or predicted to be non-targeting is flanked by loop sequences from the murine miR-155 sequence,⁵⁰ which directs the excision of the engineered miRNA from a longer Pol II transcript (pri-miRNA). All other artificial miRNA sequences made for the insertion into the miR-155 backbone were designed using the BLOCK-iT RNAi designer, a companion online tool (https://rnaidesigner.thermofisher.com/rnaiexpress/). Mature miRNA sequences were as follows: miR-neco: AAATGTACTGCGCGTGGAGAC (SEQ ID NO: 53); miR-lacZ: AAATCGCTGATTTGTGTAGTC (SEQ ID NO: 54); miR-luc1: AGCCCATATCGTTTCATAGCT (SEQ ID NO: 55); miR-luc2: ATACCTGGCAGATGGAACCTC (SEQ ID NO: 56); miR-p53-1: TCCACACGCAAATTTCCTTCC (SEQ ID NO: 57); miR-p53-2: AGTAGATTACCACTGGAGTCT (SEQ ID NO: 58); miR-p53-3: CAAACACGCACCTCAAAGCTG (SEQ ID NO: 59). In silico designed pre-miRNA cassettes were ordered by custom gene synthesis (Genewiz) and cloned between the transgene-coding sequence and the alphaviral 3′ conserved sequence elements of the STR-plasmid. Synthesis and purification of RNA were previously described.^{51; 52} Concentration, purity and integrity of synthetic RNA was assessed by spectrophotometry (NanoDrop 2000c, ThermoFisher Scientific) and capillary electrophoresis (Fragment Analyzer; Agilent).

Cell culture. Unless indicated otherwise, all growth media, and supplements were supplied by Life Technologies/Gibco, Fetal calf serum (FCS) was purchased from Sigma. BHK21 cells (ATCC; CCL-10) and derived transductants were grown in Eagle's Minimum Essential medium supplemented with 10% FCS. HDF cells (ATCC; PCS-201-010) were grown in fibroblast medium with 2% FCS and 1% fibroblast growth supplement (Fibroblast Medium Kit; Innoprot). Human foreskin fibroblasts (ATCC; SCRC-1041) were grown in MEM medium supplemented with 15% FCS, 1% Non-essential amino acids and 1% sodium pyruvate. All cells were cultivated at 37 °C in humidified atmosphere equilibrated to 5% CO2.

RNA transfection. RNA was electroporated into cells at room temperature using X-Vivo™ 15 serum-free medium (Lonza) as electroporation buffer and applying defined pulses with a square-wave electroporator (BTX ECM 830, Harvard Apparatus). BHK-21 cells and the derived transductants were electroporated at 750 V/cm with one pulse of 16 ms; HDF cells were electroporated at 625 V/cm, with 3 pulses of 16 ms interrupted by 400 ms intervals; HFF cells were electroporated with at 500 V/cm with one pulse of 24 ms. Lipofection of cells with p53 siRNA (Santa Cruz, sc29435) or control siRNA (Santa Cruz, sc-37000) were performed using Lipofectamine™ RNAiMax™ (ThermoFisher Scientific) following the manufacturer's instructions. Molarities or amounts of RNAs used in the experiments are indicated in the figure legends. After transfection, cells were incubated without refreshing medium until analysis.

Luciferase, β-galactosidase and viability assay. Firefly luciferase or β-galactosidase expression was assessed using either Bright-Glo Luciferase Assay System or Beta-Glo Assay System according to the manufacturer's instructions (Promega). Viability of transfected cells was assessed using luminescence-based method assaying ATP concentration over time (CellTiter-Glo assay; Promega) according to the instructions of the manufacturer. Relative viability was calculated by normalizing the value of each sample to the value of cells transfected without RNA. Bioluminescence (photons per second) of all assays was measured using a microplate luminescence reader Infinite M200 (Tecan Group).

Flow cytometric analysis. To determine fluorescent protein expression, transfected cells were harvested, washed once with PBS, and fixed with PBS containing 4% formaldehyde. Expression of fluorescent proteins was assessed using FACS Canto II flow cytometer and the companion FACSDiva™ software (BD Bioscience). FlowJo™ v10 software was used for further data analyses (BD Bioscience).

Quantitative real-time reverse transcriptase PCR (qRT-PCR) for mRNA and miRNA. To assess mRNA expression levels in cells, total RNA was extracted from cell lysates (RNEasy kit; Qiagen), quantified by spectroscopy (NanoDrop 2000c, ThermoFisher Scientific) and reverse transcribed with oligo (dT)₁₈ primer using the Superscript IV Reverse Transcriptase (Invitrogen). The cDNA products were diluted 1:10 with nuclease free water to serve as templates for qRT-PCR, which were performed using the ABI 7300 Real time PCR System, the companion SDS v1.4 analysis software (Applied Biosystems) and the QuantiTect SYBR Green PCR Kit (Qiagen). Protocols followed the manufacturer's instruction with 15 min at 95 °C, and 40 cycles of 30 sec at 95 °C, 30 sec at 60 °C and 30 sec at 72 °C. Analyses were performed using the 2-ACT or 2-AACT method, 53 normalized to the reference gene HPRT (HDF and HFF) or β-Actin (BHK-21 cells). The following specific primers were used: lacZ, forward: 5′-GTACGTCTTCCCGAGCGAAA-3′ (SEQ ID NO: 27), reverse: 5′-CTGTTGACTGTAGCGGCTGA-3′ (SEQ ID NO: 28); β-Actin, forward: 5′-CCTGTATGCCAACACAGTGC-3′ (SEQ ID NO: 25), reverse: 5′-ATACTCCTGCTTGCTGATCC-3′ (SEQ ID NO: SecNluc, forward: 5′-CTGGACCAAGTCCTTGAAC-3′ (SEQ ID NO: 60), reverse: 5′-CGCTCAGACCTTCATACG-3′ (SEQ ID NO: 61); TP53, forward: 5′-ACACTCGCTTCTGAATCATC-3′ (SEQ ID NO: 29), reverse: 5′-GAGACCATTCATAAGCAACG-3′ (SEQ ID NO: 30); TGFβR2, forward: 5′-TGAGTCCTTCAAGCAGACCGA-3′ (SEQ ID NO: 33), reverse: 5′-ACACACCATCTGGATGCCCTG-3′ (SEQ ID NO: 34); DAZAP2, forward: 5′-CGAACAGGAAGAGGACGAAA-3′ (SEQ ID NO: 35), reverse: 5′-CAGGGTAGGTTGGCTGTGTT-3′ (SEQ ID NO: 36); HPRT, forward: 5′-TGACACTGGCAAAACAATGCA-3′ (SEQ ID NO: 23), reverse: 5′-GGTCCTTTTCACCAGCAAGCT-3′ (SEQ ID NO: 24). To assess miRNA expression levels in cells, small RNA-containing total RNA was extracted from cell lysates (mirVana Kit, ThermoFisher Scientific), quantified by spectroscopy (NanoDrop 2000c, ThermoFisher Scientific) and reverse transcribed using miRCURY LNA RT Kit (Qiagen). The cDNA products were diluted 1:60 with nuclease free water to serve as templates for qRT-PCR using a Bio-Rad C₁₀₀₀ Touch Thermal Cycler, the companion CFX v3.1 analysis software (Bio-Rad) and the miRCURY LNA SYBR Green PCR Kit (Qiagen). Protocol followed the manufacturer's instruction with 2 min at 95 °C, and 40 cycles of 10 sec at 95 °C and 60 sec at 56 °C. Analyses were performed using standard curves for absolute quantification of pre-miR-p53-2 and 2-AACT method for relative quantification of all five miRNAs of the miR-302/367 cluster,⁵³ normalized to the reference small RNA gene SNORD48. Specific LNA-enhanced primers were custom-designed to specifically amplify mature miRNA sequences (miRCURY LNA miRNA custom PCR Assays; Qiagen) including hsa-SNORD48 (NR_002745), hsa-miR-302a-3p (TAAGTGCTTCCATGTTTTGGTGA) (SEQ ID NO: 62), hsa-miR-302b-3p (TAAGTGCTTCCATGTTTTAGTAG) (SEQ ID NO: 63), hsa-miR-302c-3p (TAAGTGCTTCCATGTTTCAGTGG) (SEQ ID NO: 64), hsa-miR-302d-3p (TAAGTGCTTCCATGTTTGAGTGT) (SEQ ID NO: 65), hsa-miR-367-3p (AATTGCACTTTAGCAATGGTGA) (SEQ ID NO: 66), and miR-p53-2 (AGTAGATTACCACTGGAGTCT) (SEQ ID NO: 58).

Western blot. Total cell extracts were generated by resolving the cell pellets in RIPA buffer and 100× Halt™ Protease and Phosphatase Inhibitor Cocktail (Thermo Scientific). Samples were incubated for 30 min at 4 °C on a rotatory-wheel followed by a centrifugation step (200×g, 4 °C for 15 min) to remove cell debris. Protein concentration in cell extracts (supernatant) was measured by Pierce BCA protein assay (ThermoFisher Scientific). Equal amounts of protein were loaded onto SDS-PAGE gels and protein transfer on nitrocellulose membrane (GE Healthcare) was performed by semi-dry western blot. Non-specific binding to the membranes was blocked with 1% (w/v) skim milk powder solutions in 1×PBS-Tween. Immunostaining with primary antibodies against p53 (Santa Cruz, sc-126) and GAPDH (GeneTex, GTX627408) was performed over night at 4 °C followed by secondary antibodies. Protein detection by chemiluminescence was performed using the Lumi-Light Western Blotting substrate (Roche) or Dura reagent (ThermoFisher Scientific), and the ImageQuant LAS 4000 detection system (GE Healthcare). Quantification of signal intensity was performed using ImageQuant TL software (GE Healthcare). To compensate for unequal sample loading, relative expression values of proteins were normalized to corresponding relative signal intensities of loading controls.

Statistics. The data of independent experiments were summarized and displayed as mean plus/minus standard deviation. All statistical analysis was performed with GraphPad Prism 9. Tests applied to the experiments are mentioned in the respective figure legends.

### Results

Incorporation of Pre-miRNA into the 3′ UTR of Protein-Coding Transreplicons Preserves High Protein Expression and Enables Target Gene Regulation

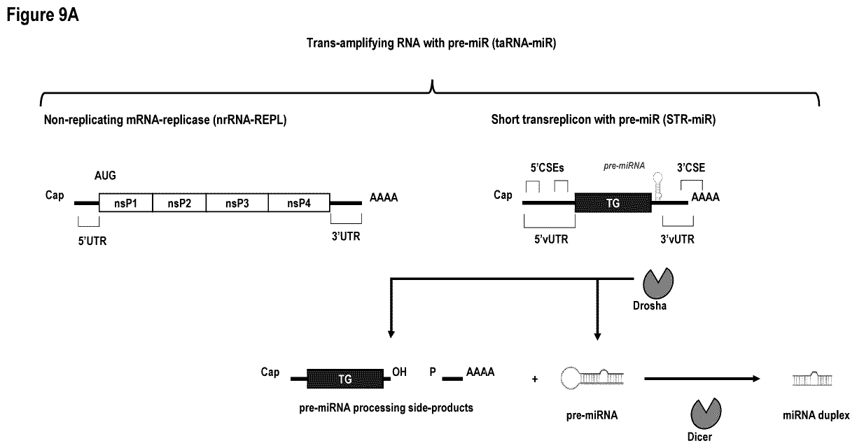

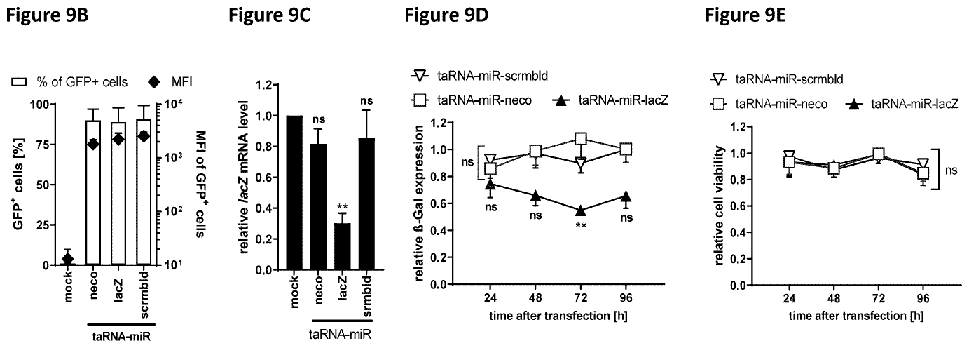

Previous studies have demonstrated that the release of functional miRNA from genomic RNA of alphaviruses that replicate in the cytoplasm follows a non-canonical miRNA biogenesis pathway.^{19; 28} Building on this knowledge, it was investigated whether functional miRNAs could be processed and released from the 3′ UTR of short transreplicons (STRs) contained within taRNA, which also replicates in the cytoplasm, and whether they would reach an effective level. To avoid the cytotoxic replicase of Semliki forest virus^{29; 30} used in previous studies,^{25; 27} taRNA was generated based on the genome of the Trinidad donkey strain (TRD) of the Venezuelan equine encephalitis virus (VEEV). Its replicase is less toxic.³¹ As done before for a number of other alphaviruses,^{25; 27; 32} the VEEV replicase was inserted into a non-replicating mRNA (nrRNA-REPL), for the transreplicon the VEEV-based STRs (FIG. 9A) were chosen.²⁷

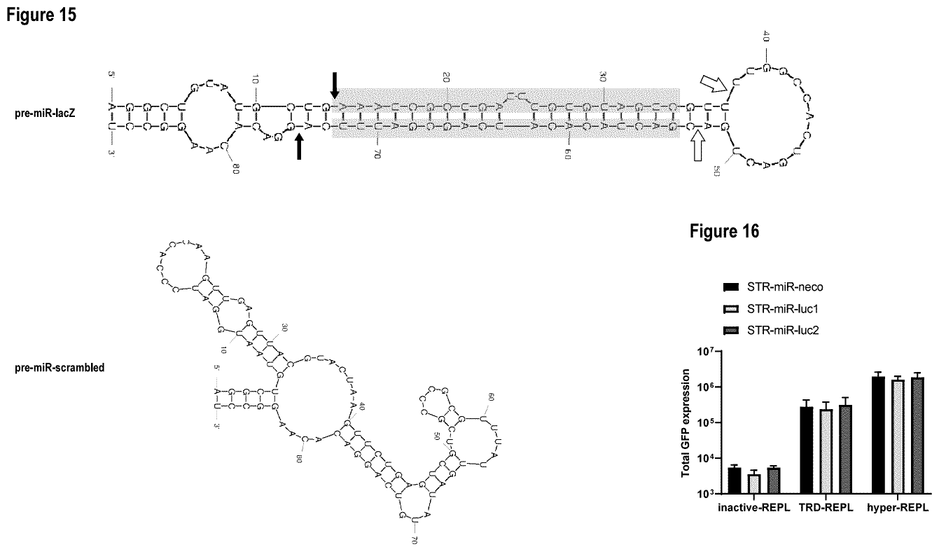

To begin with, a validated and commercially available lentiviral amiRNA expression/reporter-cassette was inserted into the STR (STR-miR). The STR-miR thereby comprised the emerald green fluorescent protein (emGFP) followed downstream by an optimized pre-miR-155 backbone⁵ containing an amiRNA against the bacterial lacZ mRNA (miR-lacZ) encoding for β-galactosidase (β-Gal). Upstream and downstream to this cassette the alphaviral 5′- and 3′-UTRs and CSEs were kept to ensure replication. Expectedly, an excision of the pre-miR hairpin from an STR-miR RNA-molecule would leave behind replication-incompetent by-products (FIG. 9A). To investigate whether miRNA processing would measurably reduce STR replication and expression, a scrambled pre-miR-lacZ control (pre-miR-srmbld) was generated to disrupt the miRNA secondary structure (FIG. 15), and prevent recognition and processing by Drosha. The available negative control was also inserted into the STR comprising an amiRNA that according to the vendor does not target any known vertebrate gene (miR-neco). For the experiments, BHK-21 cells stably transduced with the lacZ gene to express β-Gal (BHK-lacZ) were generated.

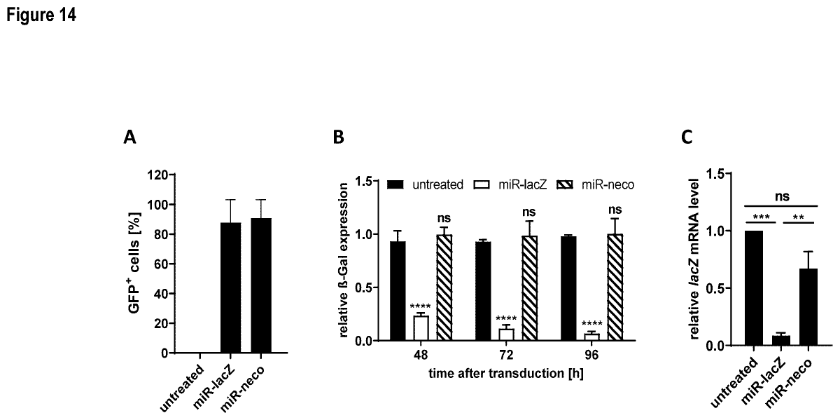

To estimate the presumably maximal achievable knockdown efficiency in this artificial experimental setting transduced BHK-lacZ cells with both purchased lentiviral miR-vectors were transduced. Thanks to very high transduction rates of approximately 90% a more than 90% knockdown efficiency of β-Gal and lacZ expression was achieved within 96 h, while the negative control did not alter expression (FIG. 14). Next, BHK-lacZ cells were transfected with nrRNA-REPL of VEEV-TRD and STR-miR (together designated taRNA-miR), resulting in comparable emGFP expression for all three STR-miR constructs (FIG. 9B). Within 72 h of transfection, taRNA-miR-lacZ significantly reduced lacZ transcript levels by 70% and ß-Gal protein levels by 50%, while expression remained unaltered in controls (FIGS. 9C and D). Moreover, taRNA-miR transfections did not hamper cell viability (FIG. 9E).

Although lentiviral vectors were more effective in silencing lacZ, these results provided first evidence that taRNA-miR can suppress target expression in a sequence-specific manner while conserving reporter gene expression and cell viability.

### Replication of STR-miR is Required for Target Knockdown and Replicase Activity Determines the Extent of Knockdown

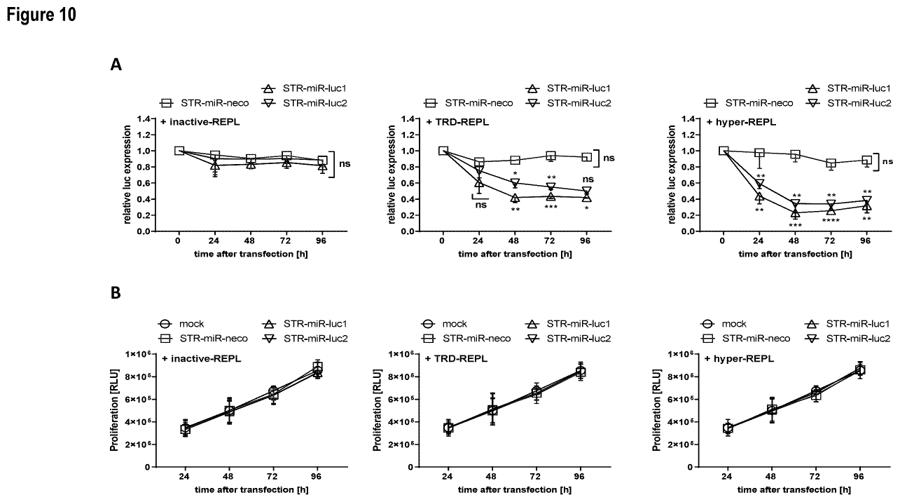

To confirm the effectual miRNA delivery using STRs and extend our analysis, the miR-lacZ was replaced by two amiRNAs targeting firefly luciferase (STR-miR-luc1, STR-miR-luc2) and BHK-21 cells were generated that stably expressed luciferase (BHK-luc). To investigate whether STR replication is required for target gene knockdown, BHK-luc was co-transfected with both STR-miR-luc constructs, along with a nrRNA encoding either the replicase of VEEV-TRD (TRD-REPL) used before, a replication-deficient mutant (inactive-REPL), or a hyperactive replicase (hyper-REPL)³³ for increased replication rates. The day after transfection, the emGFP expression level of the cells observed reflected the type of replicase used (FIG. 16). Specifically, with the inactive replicase, the capped STR-miR translated to a basal GFP expression, which was amplified more than 50 times by the TRD-REPL, and further increased about 10-fold by the hyper-REPL (FIG. 16). Regarding luciferase expression, no significant silencing was found when co-transfecting STR-miR-luc and inactive-REPL (FIG. 10A, left). Co-transfecting the TRD-REPL reduced luciferase expression by 50-60% (FIG. 10A, middle), and co-transfecting hyper-REPL culminated in 80% silencing (FIG. 10A, right). Importantly, cell viability remained unaffected regardless of the replicase variant used (FIG. 10B). It can be concluded that taRNA-miR suppresses a target gene in a replication-dependent manner without being cytotoxic.

taRNA-miR Mediates Sustained Suppression of an Endogenous Target Gene in Primary Cells and Mature miRNAs Accumulate During Replication

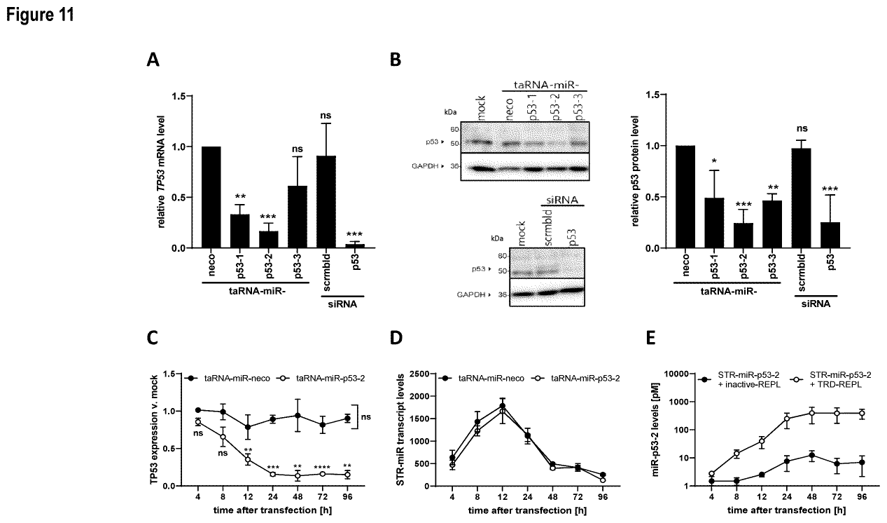

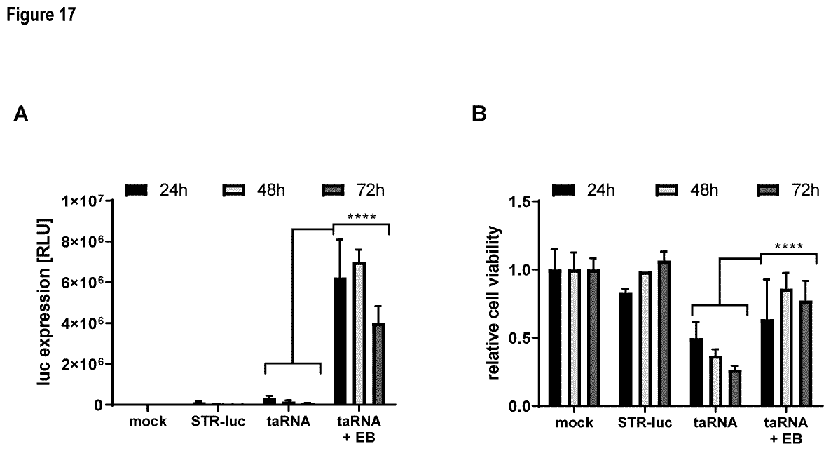

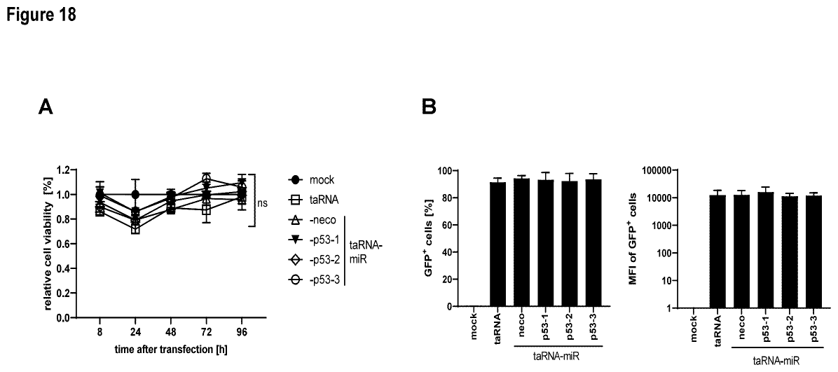

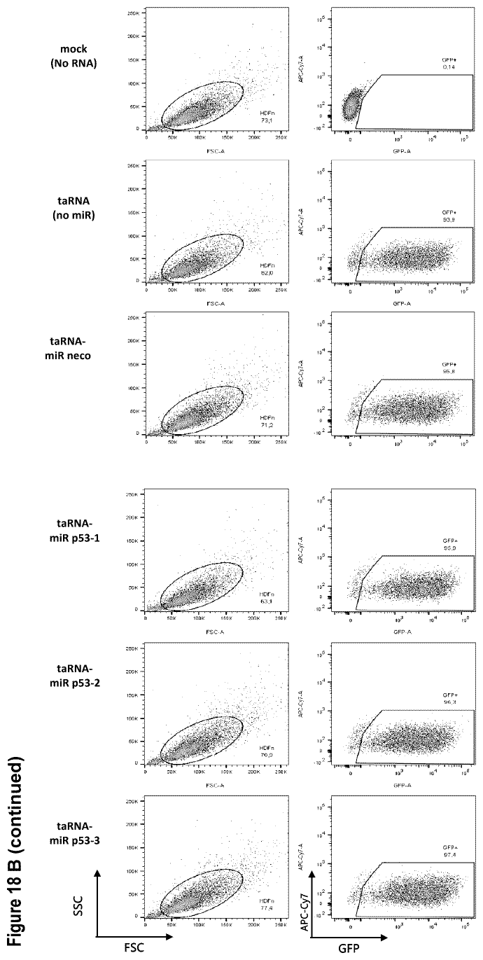

Having repeatedly achieved targeted gene knockdown by taRNA-miR in reporter cell lines, it was now aimed to downregulate an endogenous transcript under more physiologic conditions in primary human dermal fibroblasts (HDF). To this aim, three STR-miR constructs were designed expressing a dual GFP-SecNLuc reporter gene and inserted amiRNAs targeting human p53 (miR-p53-1, miR-p53-2, miR-p53-3). As controls, an STR encoding GFP-SecNLuc without the miRNA cassette and the previously used miR-neco were used. HDF possess an innate immune response that inhibits saRNA and taRNA replication,^{27; 34} which is activated by the transfection of in vitro transcribed, unmodified RNA, as well as double stranded RNA (dsRNA) intermediates generated during RNA-replication.^{35; 36} To counteract the innate immune response, cells were co-transfected with nrRNAs encoding the Vaccinia Virus (VACV) immune evasion proteins E3 and B18R (EB), which significantly enhance saRNA expression,³⁴ and were also boosting taRNA-expression and reducing cytotoxicity (FIG. 17). Hence, all subsequent taRNA-miR-p53 experiments in HDF were conducted in the presence of co-transfected EB nrRNA. It was observed that all three taRNA-miR-p53 constructs downregulated p53 expression at both the transcript and protein levels compared to the miR-neco control, with miR-p53-2 being most efficient, achieving an 80% knockdown efficiency (FIG. 11A, B). Neither viability of the transfected HDF (FIG. 18A) nor the taRNA-encoded transgene expression was impaired compared to controls (FIG. 18B).

To investigate the kinetics of TP53 gene knockdown and its correlation with STR-replication and release of mature miRNA, time-course experiments were performed using most efficient miR-p53-2. The results showed that 24 h after transfection, TP53 transcript levels were maximally reduced by 80% and remained suppressed for at least another 72 h (FIG. 11C). Concurrently, the STR-miR copy number increased within the first 12 h in response to nrRNA-REPL, and then decreased (FIG. 11D), negatively correlating with the knockdown kinetics (FIG. 11C). A good correlation between TP53 knockdown and the level of mature miRNAs released from replicating STR-miR was also observed (FIG. 11E). Both reached sustained maximal levels from around 24 to 96 h after transfection (FIGS. 11C and E). Interestingly, mature miRNA was also measurably released from STR-miR-p53-2 transfected cells with inactive-REPL, albeit at a much lower level (FIG. 11E), indicating that transfected RNAs are generally accessible for cytoplasmic pre-miR processing. To sum up, it was demonstrated that taRNA-miR suppressed an endogenous target gene in primary cells for several days, which is correlated with the presence of processed mature miRNAs.

A Polycistronic miRNA Cluster is Processed from taRNA-miR and Targeted Genes are Suppressed for Several Days

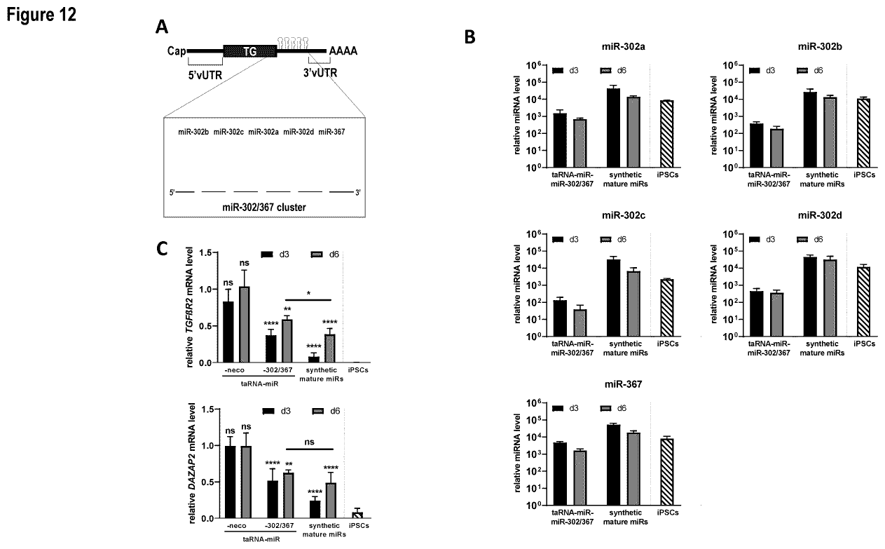

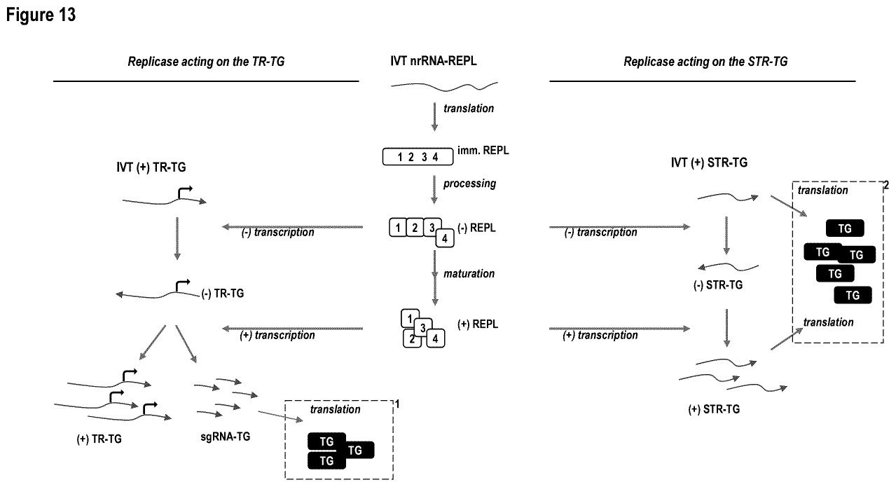

While synthetic miRNAs typically target only the specific gene of interest, natural miRNAs are known to regulate a network of target genes to different extents. Many miRNA genes are arranged in clusters⁵⁴, which further expands their pleiotropic effects. Hence, whether the taRNA-miR platform could functionally deliver a natural miRNA gene cluster and lead to the simultaneous release of multiple mature miRNAs was investigated. To address both questions, the polycistronic human miR-302/367 cluster was chosen, which contains five miRNA hairpins, namely miR-302b, -c, -a, -d and-367 and is highly expressed in human embryonic and induced pluripotent stem cells (iPSCs). This cluster regulates many genes involved in cell signaling, cell cycle, and epigenetic regulation of pluripotency.³⁷ The synthetic miR-155 backbone was replaced with the miR-302/367 cluster in the STR 3′UTR (FIG. 12A) and co-transfected HDF with either taRNA-miR-302/367 or taRNA-miR-neco, along with EB nrRNA. As a positive control for target gene regulation, cells with an equimolar mix of five synthetic mature miRNAs corresponding to the miR-302/367 cluster were also transfected. All five mature miRNAs in taRNA-miR-302/367 transfected cells three and six days after transfection were specifically detected, at levels that were approximately 100 to 1000 times higher compared to those in miR-neco transfected cells (FIG. 12B). Although the mature miRNAs released from taRNA-miR-302/367 did not reach the levels found in iPSCs (FIG. 12B), two known target genes of the cluster, DAZAP2 and TGFβR2,³⁷ were significantly suppressed in taRNA-miR-302/367 transfected HDF compared to miR-neco transfected cells, indicating that physiologically relevant levels were reached (FIG. 12C). In fact, although the transfection of synthetic miRNA achieved more than 10 times higher levels, target gene suppression was only about 30% more effective (FIG. 12C). In established iPSCs, TGFβR2 and DAZAP2 expression was barely detectable (FIG. 12C).

These findings demonstrate that the taRNA-miR platform can effectively deliver a natural polycistronic miRNA gene, resulting in the release of all enclosed miRNAs at levels sufficient for target gene regulation.

### Discussion

In this study, it was demonstrated that the taRNA vector platform can effectively deliver functional miRNAs and protein coding sequences simultaneously. It was observed that RNA replication is necessary for achieving target gene regulation, and that targets remain suppressed for several days. The incorporation of miRNA into taRNA thereby extends the functionality of this highly immunogenic vaccine vector platform.

The miRNA sequences that were incorporated into STRs correspond to pri-miRNAs, which are typically processed by nuclear Drosha as part of the canonical miRNA biogenesis pathway.³⁸ However, several studies have shown that miRNAs incorporated into the genome of RNA viruses that localize exclusively to the cytoplasm can also be processed by Drosha, yet by a cytoplasmically relocalized Drosha.^{22; 39} Moreover, alternative splicing can generate cytoplasmic isoforms of Drosha that cleave pri-miRNAs outside the nucleus,^{40; 41} suggesting altogether that STR-miRs are processed by Drosha. It was shown herein that target-specific miRNAs from STR-miRs mediate target suppression, indicating successful processing. Interestingly and importantly, it was found that target regulation did not impair the ability of STR-miRs to replicate, or to express co-encoded proteins. Actually, the released mature amiRNAs could theoretically counteract replication or translation by self-targeting complementary sequences in the STR-miR, and self-cleavage would abolish the capability of the RNA-molecules to replicate. Both does not seem to be relevant. Although self-targeting with siRNA is a natural antiviral defence mechanism found in most invertebrates and plants⁴², RNAi-mediated cleavage may be handicapped by complex secondary structures that mask the target sequence and protect it from the RISC complex.¹³ The absence of a measurable impact of STR-miR processing on STR-encoded protein expression indicates that the majority of STR-miR molecules is not processed to mature miRNA. This indicates that the high replication rates allow for sacrificing a small proportion of the transfected or de novo synthesized STR copies for miRNA production, and potential self-targeting would not significantly affect replication. This hypothesis is supported by observations made with engineered flaviviruses containing miRNA transcripts, where at least 100-fold fewer miRNAs were produced in infected cells than genomic RNA copies.¹⁸ Additionally, RNA replication takes place in membranous compartments sealed by replicase, known as spherules.⁴³ The RNA, which is template for replication localized inside the spherules, which provided an isolated and protective environment, potentially shielding it from the effects of the released miRNAs.

In the above study, the kinetics of taRNA-miR mediated target knockdown and the simultaneous accumulation of mature amiRNA correlated directly with the kinetics of STR-miR replication. Although a low number of mature miRNAs released from STRs in the absence of replicase was observed, only the amplification to high STR-miR copy numbers enabled target suppression. Conversely, producing more STR-miR transcripts by enhancing trans-replication with a hyperactive replicase led to a stronger target knockdown. The knockdown efficiency mediated by taRNA-miR was persistent for several days in this study, but it did not reach the efficiency of stable lentiviral miRNA transfer. These results suggest that the processing of cytoplasmic RNA is rather inefficient and requires RNA amplification to achieve biological effects, which is not observable when using non-replicating RNA. Thus, miRNA and protein co-transfer can be considered a unique selling point for replicating RNA compared to non-replicating mRNA.

In this study, it was observed that the miR-367 hairpin was processed most efficiently among the miRNAs in the miR-302/367 cluster. This finding is consistent with the observations made with SeV-based vectors developed for the long-term cytoplasmic production of miRNAs.¹⁷ The study showed that the SeV-vector-derived miR-367 hairpin is an exceptionally effective backbone for amiRNA production. In fact, the knockdown efficacy with miR-367 hairpin-based amiRNAs significantly outcompeted commonly used hairpins of miR-30, miR-124 and also miR-155 used in this study.

In primary cells, the innate immune response inhibits taRNA expression. Here, this issue was addressed by co-transfecting viral immune evasion proteins E3 and B18 as previously done to improve gene expression by self-amplifying RNA.³⁴ However, it may be possible to avoid the use of recombinant proteins or additional coding mRNAs by taRNA-encoded amiRNAs that downregulate RNA sensors or interferon-stimulating genes. Thus, immune-modulating amiRNAs are a more viable option for clinical translation of the platform. Overall, it has been shown herein that taRNA is a powerful tool for co-delivering therapeutic proteins and miRNAs. Incorporating them into untranslated regions of STRs is a simple and effective way to upgrade the functionality the taRNA platform, requiring only a minor elongation of the STR sequence that has no noticeable impact on the manufacturing of the in vitro transcribed RNAs.

### REFERENCES

1 Setten, R. L., Rossi, J. J., and Han, S. (2019). The current state and future directions of RNAi-based therapeutics. Nat Rev Drug Discor 18, 421-446.
2 Carthew, R. W., and Sontheimer, E. J. (2009). Origins and Mechanisms of miRNAs and siRNAs. Cell 136, 642-655.
3 Silva, J. M., Li, M. Z., Chang, K., Ge, W., Golding, M. C., Rickles, R. J., Siolas, D., Hu, G., Paddison, P. J., Schlabach, M. R., et al. (2005). Second-generation shRNA libraries covering the mouse and human genomes. Nature genetics 37, 1281-1288.
4 Zhou, H., Xia, X. G., and Xu, Z. (2005). An RNA polymerase II construct synthesizes short-hairpin RNA with a quantitative indicator and mediates highly efficient RNAi. Nucleic acids research 33, e62.
5 Chung, K.-H., Hart, C. C., Al-Bassam, S., Avery, A., Taylor, J., Patel, P. D., Vojtek, A. B., and Turner, D. L. (2006). Polycistronic RNA polymerase II expression vectors for RNA interference based on BIC/miR-155. Nucleic acids research 34, e53.
6 Boudreau, R. L., Monteys, A. M., and Davidson, B. L. (2008). Minimizing variables among hairpin-based RNAi vectors reveals the potency of shRNAs. RNA (New York, N.Y.) 14, 1834-1844.
7 Grimm, D. (2011). The dose can make the poison: lessons learned from adverse in vivo toxicities caused by RNAi overexpression. Silence 2.
8 McBride, J. L., Boudreau, R. L., Harper, S. Q., Staber, P. D., Monteys, A. M., Martins, I., Gilmore, B. L., Burstein, H., Peluso, R. W., Polisky, B., et al. (2008). Artificial miRNAs mitigate shRNA-mediated toxicity in the brain: Implications for the therapeutic development of RNAi. Proceedings of the National Academy of Sciences of the United States of America 105, 5868-5873.
9 Liang, X.-H., Hart, C. E., and Crooke, S. T. (2013). Transfection of siRNAs can alter miRNA levels and trigger non-specific protein degradation in mammalian cells. Biochimica et biophysica acta 1829, 455-468.
10 Boudreau, R. L., Martins, I., and Davidson, B. L. (2009). Artificial microRNAs as siRNA shuttles: Improved safety as compared to shRNAs in vitro and in vivo. Molecular therapy the journal of the American Society of Gene Therapy 17, 169-175.
11 Borel, F., van Logtenstein, R., Koornneef, A., Maczuga, P., Ritsema, T., Petry, H., van Deventer, S. J., Jansen, P. L., and Konstantinova, P. (2011). In vivo knock-down of multidrug resistance transporters ABCC1 and ABCC2 by AAV-delivered shRNAs and by artificial miRNAs. J RNAi Gene Silencing 7, 434-442.
12 Bumcrot, D., Manoharan, M., Koteliansky, V., and Sah, D. W. Y. (2006). RNAi therapeutics: A potential new class of pharmaceutical drugs. Nature chemical biology 2, 711-719.
13 Herrera-Carrillo, E., Liu, Y. P., and Berkhout, B. (2017). Improving miRNA Delivery by Optimizing miRNA Expression Cassettes in Diverse Virus Vectors. Human gene therapy methods 28, 177-190.
14 Vargas, J. E., Chicaybam, L., Stein, R. T., Tanuri, A., Delgado-Cañedo, A., and Bonamino, M. H. (2016). Retroviral vectors and transposons for stable gene therapy: advances, current challenges and perspectives. J Transl Med. 14, 288.
15 Stoller, M. L., Chang, H. C., and Fekete, D. M. (2013). Bicistronic gene transfer tools for delivery of miRNAs and protein coding sequences. Int J Mol Sci. 14, 18239-18255.
16 Park, T. S., Abate-Daga, D., Zhang, L., Zheng, Z., and Morgan, R. A. (2014). Gamma-retroviral vector design for the co-expression of artificial microRNAs and therapeutic proteins 24, 356-363.
17 Sano, M., Nakasu, A., Ohtaka, M., and Nakanishi, M. (2019). A Sendai Virus-Based Cytoplasmic RNA Vector as a Novel Platform for Long-Term Expression of MicroRNAs. Molecular therapy. Methods & clinical development 15, 371-382.
18 Rouha, H., Thurner, C., and Mandl, C. W. (2010). Functional microRNA generated from a cytoplasmic RNA virus. Nucleic acids research 38, 8328-8337.
19 Shapiro, J. S., Varble, A., Pham, A. M., and tenOever, B. R. (2010). Noncanonical cytoplasmic processing of viral microRNAs. RNA (New York, N.Y.) 16, 2068-2074.
20 Honda, T., Yamamoto, Y., Daito, T., Matsumoto, Y., Makino, A., and Tomonaga, K. (2016). Long-term expression of miRNA for RNA interference using a novel vector system based on a negative-strand RNA virus. Sci. Rep. 6.
21 Payne, S. (2017). Introduction to RNA Viruses. Viruses, 97-105.
22 Shapiro, J. S., Langlois, R. A., Pham, A. M., and tenOever, B. R. (2012). Evidence for a cytoplasmic microprocessor of pri-miRNAs. RNA (New York, N.Y.) 18, 1338-1346.
23 Lundstrom, K. (2019). RNA Viruses as Tools in Gene Therapy and Vaccine Development. Genes (Basel) 10.
24 Poleganov, M. A., Eminli, S., Beissert, T., Herz, S., Moon, J.-I., Goldmann, J., Beyer, A., Heck, R., Burkhart, I., Barea Roldan, D., et al. (2015). Efficient Reprogramming of Human Fibroblasts and Blood-Derived Endothelial Progenitor Cells Using Nonmodified RNA for Reprogramming and Immune Evasion. Human gene therapy 26, 751-766.
25 Beissert, T., Perkovic, M., Vogel, A., Erbar, S., Walzer, K. C., Hempel, T., Brill, S., Haefner, E., Becker, R., Türeci, O., et al. (2020). A Trans-amplifying RNA Vaccine Strategy for Induction of Potent Protective Immunity. Molecular therapy the journal of the American Society of Gene Therapy 28, 119-128.
26 Schmidt, C., Haefner, E., Gerbeth, J., Beissert, T., Sahin, U., Perkovic, M., and Schnierle, B. S. (2022). A taRNA vaccine candidate induces a specific immune response that protects mice against Chikungunya virus infections. Molecular therapy. Nucleic acids 28, 743-754.
27 Perkovic, M., Gawletta, S., Hempel, T., Brill, S., Nett, E., Sahin, U., and Beissert, T. (2023). A trans-amplifying RNA simplified to essential elements is highly replicative and robustly immunogenic in mice. Molecular therapy the journal of the American Society of Gene Therapy.
28 Langlois, R. A., Shapiro, J. S., Pham, A. M., and tenOever, B. R. (2012). In vivo delivery of cytoplasmic RNA virus-derived miRNAs. Molecular therapy the journal of the American Society of Gene Therapy 20, 367-375.
29 Liljestrom, P., and Garoff, H. (1991). A new generation of animal cell expression vectors based on the Semliki Forest virus replicon. Biotechnology (N.Y.) 9, 1356-1361.
30 Petrakova, O., Volkova, E., Gorchakov, R., Paessler, S., Kinney, R. M., and Frolov, I. (2005). Noncytopathic replication of Venezuelan equine encephalitis virus and eastern equine encephalitis virus replicons in Mammalian cells. Journal of virology 79, 7597-7608.
31 Garmashova, N., Gorchakov, R., Volkova, E., Paessler, S., Frolova, E., and Frolov, I. (2007). The Old World and New World alphaviruses use different virus-specific proteins for induction of transcriptional shutoff. Journal of virology 81, 2472-2484.
32 Schmidt, C., Hastert, F. D., Gerbeth, J., Beissert, T., Sahin, U., Perkovic, M., and Schnierle, B. S. (2022). A Bivalent Trans-Amplifying RNA Vaccine Candidate Induces Potent Chikungunya and Ross River Virus Specific Immune Responses. Vaccines 10, 1374.
33 Michel, G., Petrakova, O., Atasheva, S., and Frolov, I. (2007). Adaptation of Venezuelan equine encephalitis virus lacking 51-nt conserved sequence element to replication in mammalian and mosquito cells. Virology 362, 475-487.
34 Beissert, T., Koste, L., Perkovic, M., Walzer, K. C., Erbar, S., Selmi, A., Diken, M., Kreiter, S., Türeci, Ö., and Sahin, U. (2017). Improvement of In Vivo Expression of Genes Delivered by Self-Amplifying RNA Using Vaccinia Virus Immune Evasion Proteins. Human gene therapy 28, 1138-1146.
35 Gorchakov, R., Garmashova, N., Frolova, E., and Frolov, I. (2008). Different types of nsP3-containing protein complexes in Sindbis virus-infected cells. Journal of virology 82, 10088-10101.
36 Frolova, E. I., Gorchakov, R., Pereboeva, L., Atasheva, S., and Frolov, I. (2010). Functional Sindbis virus replicative complexes are formed at the plasma membrane. Journal of virology 84, 11679-11695.
37 Gao, Z., Zhu, X., and Dou, Y. (2015). The miR-302/367 cluster: A comprehensive update on its evolution and functions. Open biology 5, 150138.
38 Ha, M., and Kim, V. N. (2014). Regulation of microRNA biogenesis. Nature reviews. Molecular cell biology 15, 509-524.
39 Shapiro, J. S. (2013). Processing of virus-derived cytoplasmic primary-microRNAs. Wiley interdisciplinary reviews. RNA 4, 463-471.
40 Link, S., Grund, S. E., and Diederichs, S. (2016). Alternative splicing affects the subcellular localization of Drosha. Nucleic acids research 44, 5330-5343.
41 Dai, L., Chen, K., Youngren, B., Kulina, J., Yang, A., Guo, Z., Li, J., Yu, P., and Gu, S. (2016). Cytoplasmic Drosha activity generated by alternative splicing. Nucleic acids research 44, 10454-10466.
42 Ratcliff, F. G., MacFarlane, S. A., and Baulcombe, D. C. (1999). Gene silencing without DNA. rna-mediated cross-protection between viruses. The Plant cell 11, 1207-1216.
43 Paul, D., and Bartenschlager, R. (2013). Architecture and biogenesis of plus-strand RNA virus replication factories. World J Virol. 2, 32-48.
44 Sun, D., Melegari, M., Sridhar, S., Rogler, C. E., and Zhu, L. (2006). Multi-miRNA hairpin method that improves gene knockdown efficiency and provides linked multi-gene knockdown. BioTechniques 41, 59-63.
45 Hu, T., Fu, Q., Chen, P., Ma, L., Sin, O., and Guo, D. (2009). Construction of an artificial MicroRNA expression vector for simultaneous inhibition of multiple genes in mammalian cells. International journal of molecular sciences 10, 2158-2168.
46 Fellmann, C., Hoffmann, T., Sridhar, V., Hopfgartner, B., Muhar, M., Roth, M., Lai, D. Y., Barbosa, I. A. M., Kwon, J. S., Guan, Y., et al. (2013). An optimized microRNA backbone for effective single-copy RNAi. Cell reports 5, 1704-1713.
47 Fowler, D. K., Williams, C., Gerritsen, A. T., and Washbourne, P. (2016). Improved knockdown from artificial microRNAs in an enhanced miR-155 backbone: A designer's guide to potent multi-target RNAi. Nucleic acids research 44, e48.
48 Grimm, D., Wang, L., Lee, J. S., Schürmann, N., Gu, S., Börner, K., Storm, T. A., and Kay, M. A. (2010). Argonaute proteins are key determinants of RNAi efficacy, toxicity, and persistence in the adult mouse liver. The Journal of clinical investigation 120, 3106-3119.
49 Spuul, P., Balistreri, G., Hellstrom, K., Golubtsov, A. V., Jokitalo, E., and Ahola, T. (2011). Assembly of alphavirus replication complexes from RNA and protein components in a novel trans-replication system in mammalian cells. J. Virol. 85, 4739-4751.
50 Lagos-Quintana, M., Rauhut, R., Yalcin, A., Meyer, J., Lendeckel, W., and Tuschl, T. (2002). Identification of tissue-specific microRNAs from mouse. Current biology CB 12, 735-739.
51 Holtkamp, S., Kreiter, S., Selmi, A., Simon, P., Koslowski, M., Huber, C., Tureci, O., and Sahin, U. (2006). Modification of antigen-encoding RNA increases stability, translational efficacy, and T-cell stimulatory capacity of dendritic cells. Blood 108, 4009-4017.
52 Kreiter, S., Konrad, T., Sester, M., Huber, C., Tureci, O., and Sahin, U. (2007). Simultaneous ex vivo quantification of antigen-specific CD4+ and CD8+ T cell responses using in vitro transcribed RNA. Cancer Immunol. Immunother. 56, 1577-1587.
53 Livak, K. J., and Schmittgen, T. D. (2001). Analysis of relative gene expression data using real-time quantitative PCR and the 2 (-Delta Delta C (T)) Method. Methods 25, 402-408.
54 Gebert, L. F. R. and MacRae, I. J. (2019). Regulation of microRNA function in animals. Nat. Rev. Mol. Cell Biol. 20, 21-37

We claim:

1. A system comprising two RNA molecules,
wherein the first RNA molecule comprises an open reading frame encoding a functional RNA-dependent RNA polymerase (replicase) and
wherein the second RNA molecule is a replicable RNA molecule comprising at least one miRNA sequence, which miRNA sequence is capable of being excised from the second replicable RNA molecule when present in a cell, and is capable of regulating gene expression in a cell, and which replicable RNA molecule is capable of being replicated in trans by the replicase encoded by the first RNA molecule.

2. The system according to claim 1, wherein the second RNA molecule comprises at least one pre-miRNA sequence.

3. The system according to claim 1 or 2, wherein the first and/or second RNA molecule further comprises at least one open reading frame (ORF) encoding a protein of interest.

4. The system according to any one of claims 1 to 3, wherein the first RNA molecule is a replicable RNA molecule that can be replicated by its encoded replicase.

5. The system according to any one of claims 1 to 3, wherein the first RNA molecule is not a replicable RNA molecule.

6. The system according to any one of claims 1 to 3 or 5, wherein the first RNA molecule is an mRNA.

7. The system according to any one of claims 1 to 6, wherein the replicase is derived from the functional non-structural protein from a self-replicating virus.

8. The system according to claim 7, wherein the self-replicating virus is an alphavirus, preferably selected from the group consisting of Venezuelan equine encephalitis virus, Eastern equine encephalitis virus, Western equine encephalitis virus, Chikungunya virus, Semliki Forest virus, Sindbis virus, Barmah Forest virus, Middelburg virus and Ndumu virus.

9. The system according to claim 8, wherein the alphavirus is Venezuelan equine encephalitis virus or Semliki Forest virus.

10. The system according to any one of claims 1 to 9, wherein the second RNA molecule comprises at least two, at least three, at least four, at least five, or at least ten miRNA sequences.

11. The system according to claim 10, wherein the sequence of at least one miRNA sequence differs in its sequence from the other miRNA sequences, preferably wherein the sequence of each miRNA differs from the other.

12. The system according to claim 10, wherein the sequences of the miRNAs are the same sequence.

13. The system according to any one of claims 10 to 12, wherein the miRNAs target the same mRNA.

14. The system according to any one of claims 10 to 12, wherein the miRNAs target different mRNAs.

15. The system according to any one of claims 10 to 12, wherein the miRNAs target different sites on the same mRNA, or wherein the miRNAs target different sites on two or more mRNAs.

16. The system according to any one of claims 1 to 15, wherein the miRNA sequence is a naturally occurring miRNA sequence, preferably a human miRNA sequence.

17. The system according to any one of claims 1 to 15, wherein the miRNA sequence is an artificial miRNA sequence.

18. The system according to any one of claims 1 to 17, wherein the miRNA is a non-viral miRNA.

19. The system according to any one of claims 1 to 18, wherein the miRNA is a stem cell specific miRNA.

20. The system according to any one of claims 1 to 18, wherein the miRNA suppresses an innate immune response.

21. The system according to any one of claims 1 to 18, wherein the target of the miRNA is an mRNA that is relevant for the onset or progression of a disease, preferably an mRNA of an oncogene, mutated tumor suppressor gene or of a viral, bacterial or fungal gene.

22. The system according to claim 21, wherein the target of the miRNA is a mutated tumor suppressor gene.

23. The system according to claim 22, wherein the mutated tumor suppressor gene is TP53.

24. The system according to any one of claims 1 to 21, wherein the target of the miRNA is an interferon stimulated gene, preferably RSAD2 (viperin).

25. The system according to any one of claims 1 to 21, wherein the target of the miRNA is retinoic acid-inducible gene I (RIG-I).

26. The system according to any one of claims 1 to 21, wherein the target of the miRNA is the Eukaryotic Translation Initiation Factor 2 Alpha Kinase 2 (EIF2AK2) gene encoding protein kinase R (PKR).

27. The system according to any one of claims 1 to 21, wherein the target of the miRNA is DAZ-associated protein 2 (DAZAP2) and/or TGF beta receptor 2 (TGFβR2).

28. The system according to any one of claims 1 to 27, wherein the sequence of the miRNA comprises flanking and loop sequences from a naturally occurring miRNA, preferably from murine miR-155.

29. The system according to any one of claims 1 to 19, wherein the miRNA sequence is at least one miRNA sequence of the miR-302/367 cluster.

30. The system according to any one of claims 3 to 29, wherein the ORF is flanked by a 5′ untranslated region (UTR) and/or 3′ UTR.

31. The system according to any one of claims 3 to 30, wherein the protein of interest is a reporter protein, preferably GFP or a variant thereof.

32. The system according to any one of claims 3 to 30, wherein the protein of interest is a pluripotency factor or a differentiation factor.

33. The system according to any one of claims 3 to 30, wherein the protein of interest is an antigen or epitope thereof, preferably a T cell epitope.

34. The system according to claim 33, wherein the antigen or epitope is or is derived from a bacterial, viral, parasitical or fungal antigen.

35. The system according to any one of claims 3 to 30, wherein the protein of interest is a Vaccinia virus immune evasion protein.

36. The system according to claim 35, wherein the protein of interest is E3 or B18.

37. The system according to any one of claims 3 to 36, wherein the sequences of the miRNA is located in the 3′ untranslated region (UTR) of the at least one ORF of the second RNA molecule.

38. The system according to any one of claims 3 to 38, wherein the 5′-end of the miRNA sequences is connected to the ORF by a linker sequence, and/or the 3′-end of the miRNA sequences is connected to the 3′ conserved sequence element of the 3′UTR of the second RNA molecule by a linker sequence.

39. The system according to any one of claims 3 to 39, wherein each of the miRNA sequences are connected by a linker sequence.

40. The system according to claim 38 or 39, wherein the linker sequence comprises 5 to 30 nucleotides.

41. The system according to any one of claims 1 to 40, wherein the first and/or second RNA molecule is a modified RNA molecule.

42. The system according to claim 41, wherein the first and/or second RNA molecule is a modified RNA molecule comprising at least one modified uridine.

43. The system according to claim 42, wherein at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the uridines in the RNA molecules are pseudouridine (ψ), N1-methyl-pseudouridine (m1ψ), or 5-methyl-uridine (m5U), preferably N1-methyl-pseudouridine (1 ml).

44. The system according to any one of claims 1 to 43, wherein the first and/or second RNA molecule further comprises a 5′ cap, a 5′ regulatory region, a 5′ replication recognition sequence, a 3′ replication recognition sequence and/or a poly(A) sequence.

45. The system according to any one of claims 1 to 44, wherein the first and/or second RNA molecule comprises a 5′ cap, which is a naturally occurring 5′ cap or a 5′ cap analog.

46. The system according to claim 45, wherein the 5′ cap analog is one of ARCA, beta-S-ARCA, beta-S-ARCA (D1), beta-S-ARCA (D2), CleanCap, Cap0, Cap1 or AU (Cap1).

47. The system according to any one of claims 1 to 46, wherein the first and/or second RNA molecule comprises at least one modified uridine and wherein the RNA molecules comprises a 5′ cap having the sequence NpppNU, wherein the U in the 5′ cap is an unmodified uridine.

48. The system according to claim 47, wherein the 5′ cap has the sequence NpppAU with A representing a modified or unmodified adenosine nucleotide.

49. The system according to any one of claims 1 to 48, wherein the first and/or second RNA molecule comprises a 5′ cap comprising a Cap1 and a cap proximal sequence comprising positions+1, +2, +3, +4, and +5 of the RNA molecule(s), wherein:
(i) the Cap1 comprises m7G (5′)ppp(5′) (2′OMeN1) pN2, wherein N1 is position+1 of the RNA molecule, and N2 is position+2 of the RNA molecule, and wherein N1 and N2 are each independently chosen from: A, C, G, or U; and
(ii) the cap proximal sequence comprises N₁ and N₂ of the Cap1, and:
(a) a sequence selected from the group consisting of: A₃A₄X₅; C₃A₄X₅; A₃C₄A₅ and A₃U₄G₅; or
(b) a sequence comprising: X₃Y₄X₅;
wherein X₃ or X₅ is each independently chosen from A, G, C, or U; and wherein Y₄ is not C.

50. The system according to any one of claims 1 to 49, wherein the first and/or second RNA molecule comprises a modified 5′ regulatory region of a self-replicating RNA virus, which modified regulatory region comprises a point mutation at one or more of positions 67, 244, 245, 246, 248 of the 5′ regulatory region (SEQ ID NO: 1).

51. The system according to claim 50, wherein the self-replicating RNA virus is an alphavirus.

52. The system according to claim 50 or 51, wherein the 5′ regulatory region further comprises a point mutation at position 4 of the 5′ regulatory region (SEQ ID NO: 1).

53. The system according to any one of claims 50 to 52, wherein the point mutation is G4A, A67C, G244A, C245A, G246A, or C248A.

54. The system according to any one of claims 1 to 53, wherein the first and/or second RNA molecule comprises a 5′ replication recognition sequence, which is characterized in that at least one initiation codon is removed compared to a native 5′ replication recognition sequence.

55. The system according to claim 54, wherein the 5′ replication recognition sequence comprises a sequence homologous to an open reading frame of a non-structural protein or a portion thereof from a self-replicating virus, wherein the sequence homologous to an open reading frame of a non-structural protein or a portion thereof from a self-replicating virus is characterized in that it comprises the removal of at least one initiation codon compared to the native viral sequence.

56. The system according to claim 55, wherein the sequence homologous to an open reading frame of a non-structural protein or a portion thereof from a self-replicating virus is characterized in that it comprises the removal of at least the native start codon of the open reading frame of a non-structural protein from a self-replicating virus.

57. The system according to claim 55 or 56, wherein the sequence homologous to an open reading frame of a non-structural protein or a portion thereof from a self-replicating virus is characterized in that it comprises the removal of at least one initiation codon other than the native start codon of the open reading frame of a non-structural protein from a self-replicating virus.

58. The system according to any one of claims 55 to 57, wherein the sequence homologous to an open reading frame of a non-structural protein or a portion thereof from a self-replicating virus is characterized in that it is free of initiation codons.

59. The system according to any one of claims 55 to 58, wherein the first and/or second RNA molecule comprises at least one nucleotide change compensating for nucleotide pairing disruptions within at least one stem loop introduced by the removal of at least one initiation codon.

60. The system according to any one of claims 1 to 59, wherein the first and/or second RNA molecule comprises a 3′ replication recognition sequence.

61. The system according to any one of claims 1 to 60, wherein the 5′ and/or 3′ replication recognition sequences are derived from a self-replicating virus, preferably the same self-replicating virus species.

62. The system according to any one of claims 1 to 61, wherein the first and/or second RNA molecule comprises a poly(A) sequence comprising from about 80 to about 150 A residues, or an interrupted poly(A) sequence.

63. The system according to any one of claims 1 to 62, wherein the first and/or second RNA molecule does not comprise an open reading frame for an intact virus structural protein.

64. The system according to any one of claims 1 to 63 further comprising a third or more replicable RNA molecules that can be replicated by the replicase encoded by the first RNA molecule.

65. The system according to any one of claims 1 to 64 further comprising a reagent capable of forming particles with at least one of the RNA molecules.

66. The system according to claim 65, wherein the reagent is or comprises a polyalkyleneimine or a lipid.

67. The system according to claim 65 or 66, wherein the reagent is or comprises a lipid, preferably comprising a cationic headgroup.

68. The system according to any one of claims 65 to 67, wherein the reagent is or comprises a pH responsive lipid.

69. The system according to any one of claims 65 to 68, wherein the reagent is or comprises a PEGylated-lipid.

70. The system according to any one of claims 65 to 69, wherein the reagent is conjugated to polysarcosine, optionally wherein the reagent comprises a lipid conjugated to polysarcosine.

71. The system according to any one of claims 65 to 70, wherein the particles formed from at least one of the RNA molecules and the reagent are polymer-based polyplexes (PLX), lipid nanoparticles (LNP), lipoplexes (LPX) or liposomes.

72. The system according to any one of claims 65 to 71, wherein the particle further comprises at least one phosphatidylserine.

73. The system according to any one of claims 65 to 72, wherein the particles are nanoparticles, in which:
(i) the number of positive charges in the nanoparticles does not exceed the number of negative charges in the nanoparticles and/or
(ii) the nanoparticles have a neutral or net negative charge and/or
(iii) the charge ratio of positive charges to negative charges in the nanoparticles is 1.4:1 or less and/or
(iv) the zeta potential of the nanoparticles is 0 or less.

74. The system according to claim 73, wherein the charge ratio of positive charges to negative charges in the nanoparticles is between 1.4:1 and 1:8, preferably between 1.2:1 and 1:4.

75. The system according to claim 73 or 74, wherein the nanoparticles comprise at least one lipid, preferably comprise at least one cationic lipid.

76. The system according to claim 75, wherein the positive charges are contributed by the at least one cationic lipid and the negative charges are contributed by the RNA molecule.

77. The system according to claim 75 or 76, wherein the nanoparticles further comprise at least one helper lipid.

78. The system according to claim 77, wherein the helper lipid is a neutral lipid.

79. The system according to any one of claims 75 to 78, wherein the at least one cationic lipid comprises 1,2-di-O-octadecenyl-3-trimethylammonium propane (DOTMA), 1,2-dioleyloxy-3-dimethylaminopropane (DODMA), and/or 1,2-dioleoyl-3-trimethylammonium-propane (DOTAP).

80. The system according to any one of claims 77 to 79, wherein the at least one helper lipid comprises 1,2-di-(9Z-octadecenoyl)-sn-glycero-3-phosphoethanolamine (DOPE), cholesterol (Chol), 1,2-dioleoyl-sn-glycero-3-phosphocholine (DOPC), and/or 1,2-distearoyl-sn-glycero-3-phosphocholine (DSPC).

81. The system according to any one of claims 77 to 80, wherein the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1.

82. The system according to any one of claims 73 to 81, wherein the nanoparticles are lipoplexes comprising DODMA and DOPE in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

83. The system according to any one of claims 73 to 81, wherein the nanoparticles are lipoplexes comprising DODMA and Cholesterol in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

84. The system according to any one of claims 73 to 81, wherein the nanoparticles are lipoplexes comprising DODMA and DSPC in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

85. The system according to any one of claims 73 to 81, wherein the nanoparticles are lipoplexes comprising DODMA:Cholesterol:DOPE:PEGcerC16 in a molar ratio of 40:48:10:2.

86. The system according to any one of claims 73 to 81, wherein the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

87. The system according to any one of claims 73 to 81, wherein the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

88. The system according to any one of claims 73 to 81, wherein the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

89. The system according to any one of claims 65 to 88, wherein the reagent comprises a lipid and the particles formed are LNPs which are complexed with and/or encapsulate the RNA molecules.

90. The system according to any one of claims 65 to 89, wherein the reagent comprises a lipid and the particles formed are vesicles encapsulating the RNA molecules, preferably unilamellar liposomes.

91. The system according to claim 65 or 66, wherein the reagent is or comprises polyalkyleneimine.

92. The system according to claim 90, wherein (a) the molar ratio of the number of nitrogen atoms (N) in the polyalkyleneimine to the number of phosphor atoms (P) in the RNA molecules (N:P ratio) is 2.0 to 15.0, preferably 6.0 to 12.0; or (b) the molar ratio of the number of the number of nitrogen atoms (N) in the polyalkyleneimine to the number of phosphor atoms (P) in the RNA molecules (N:P ratio) is at least about 48, optionally about 48 to 300, about 60 to 200, or about 80 to 150.

93. The system according to claim 90 or 91, wherein the ionic strength of the composition is 50 mM or less, preferably wherein the concentration of monovalent cationic ions is 25 mM or less and the concentration of divalent cationic ions is 20 UM or less.

94. The system according to any one of claims 91 to 93, wherein the particles formed are polyplexes.

95. The system according to any one of claims 91 to 94, wherein the polyalkyleneimine comprises the following general formula (I): 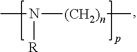
wherein
R is H, an acyl group or a group comprising the following general formula (II): 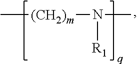
wherein R¹ is H or a group comprising the following general formula (III): 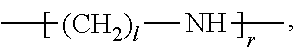
n, m, and l are independently selected from integers from 2 to 10; and
p, q, and r are integers, wherein the sum of p, q, and r is such that the average molecular weight of the polymer is 1.5.10² to 10⁷ Da, preferably 5000 to 10⁵ Da, more preferably 10000 to 40000 Da, more preferably 15000 to 30000 Da, even more preferably 20000 to 25000 Da.

96. The system according to claim 95, wherein n, m, and l are independently selected from 2, 3, 4, and 5, preferably from 2 and 3.

97. The system according to claim 95 or 96, wherein R¹ is H.

98. The system according to any one of claims 95 to 97, wherein R is H or an acyl group.

99. The system according to any one of claims 95 to 98, wherein the polyalkyleneimine comprises polyethylenimine and/or polypropylenimine, preferably polyethyleneimine.

100. The system according to any one of claims 98 to 99, wherein at least 92% of the N atoms in the polyalkyleneimine are protonatable.

101. The system according to any one of claims 1 to 100 further comprising one or more peptide-based adjuvants, wherein peptide-based adjuvants optionally comprise immune regulatory molecules, such as cytokines, lymphokines and/or co-stimulatory molecules.

102. The system according to any one of claims 1 to 101 further comprising one or more additives, wherein the additives optionally are selected from the group consisting of buffering substances, saccharides, stabilizers, cryoprotectants, lyoprotectants, and chelating agents.

103. The system according to claim 102, wherein the buffering substances comprise at least one selected from the group consisting of 4-(2-hydroxyethyl)-1-piperazineethanesulfonic acid (HEPES), 2-(N-morpholino) ethanesulfonic acid (MES), 3-morpholino-2-hydroxypropanesulfonic acid (MOPSO), acetic acid, acetate buffers and analogues, phosphoric acid and phosphate buffers, and citric acid and citrate buffers.

104. The system according to claim 102 or 103, wherein the saccharides comprise at least one selected from the group consisting of monosaccharides, disaccharides, trisaccharides, oligosaccharides, and polysaccharides preferably from glucose, trehalose, and saccharose.

105. The system according to any one of claims 102 to 104, wherein the cryoprotectants comprise at least one selected from the group consisting of glycols, such as ethylene glycol, propylene glycol, and glycerol.

106. The system according to any one of claims 102 to 105, wherein the chelating agent comprises EDTA.

107. A kit comprising two RNA molecules,
wherein the first RNA molecule comprises an open reading frame encoding a functional RNA-dependent RNA polymerase (replicase) and
wherein the second RNA molecule is a replicable RNA molecule comprising at least one miRNA sequence, which miRNA sequence is capable of being excised from the second replicable RNA when present in a cell, and is capable of regulating gene expression in a cell, which replicable RNA molecule is capable of being replicated in trans by the replicase encoded by the first RNA molecule.

108. The kit according to claim 107, wherein the first and/or second RNA molecule, preferably the second RNA molecule, further comprises at least one open reading frame (ORF) encoding a protein of interest.

109. The kit according to claim 107 or 108, wherein the two RNA molecules are in separate containers.

110. A pharmaceutical composition comprising two RNA molecules,
wherein the first RNA molecule comprises an open reading frame encoding a functional RNA-dependent RNA polymerase (replicase) and
wherein the second RNA molecule is a replicable RNA molecule comprising at least one miRNA sequence, which miRNA sequence is capable of being excised from the second replicable RNA when present in a cell, and is capable of regulating gene expression in a cell, which replicable RNA molecule is capable of being replicated in trans by the replicase encoded by the first RNA molecule, and a pharmaceutically acceptable carrier.

111. The pharmaceutical composition according to claim 110, wherein the first and/or second RNA molecule, preferably the second RNA molecule, further comprises at least one open reading frame (ORF) encoding a protein of interest.

112. The pharmaceutical composition according to claim 110 or 111, which is formulated for intradermal, subcutaneous, and/or intramuscular administration, such as by injection.

113. The kit or the pharmaceutical composition according to any one of claims 107 to 112 for use in therapy.

114. The kit or the pharmaceutical composition according to any one of claims 107 to 113 for use in a method of treating or preventing a disease, preferably wherein the subject is a mammal, more preferably wherein the mammal is a human, said method comprising administering the kit or the pharmaceutical composition according to any one of claims 107 to 112 to the subject.

115. The kit or the pharmaceutical composition for use according to claim 114, wherein administering the pharmaceutical composition comprises intradermal, subcutaneous, or intramuscular administration, such as by intradermal, subcutaneous or intramuscular injection.

116. The kit or the pharmaceutical composition for use according to claim 114, wherein the injection is by use of a needle or is by use of a needleless injection device.

117. The kit or the pharmaceutical composition for use according to any one of claims 114 to 116, wherein administering comprises administration by intramuscular injection, preferably with a needle.

118. The kit or the pharmaceutical composition for use according to any one of claims 114 to 117, wherein the RNA molecules are administered separately, preferably by the same route of administration.

119. The kit or the pharmaceutical composition for use according to any one of claims 114 to 117, wherein the disease is a bacterial, viral, parasitical or fungal infection, a cardiovascular disease, or cancer in a subject.

120. A method for the treatment or prevention of a bacterial, viral, parasitical or fungal infection in a subject, said method comprising administering to the subject a kit or a composition according to any one of claims 107 to 119.

121. A method for the treatment or prevention of cancer in a subject, said method comprising administering to the subject a kit or a composition according to any one of 107 to 119.

122. A first RNA molecule and a second RNA molecule for use in therapy, wherein the first RNA molecule comprises an open reading frame encoding a functional RNA-dependent RNA polymerase (replicase) and the second RNA molecule is a replicable RNA molecule comprising at least one miRNA sequence, which miRNA sequence is capable of being excised from the second replicable RNA when present in a cell, and is capable of regulating gene expression in a cell, and which replicable RNA molecule is capable of being replicated in trans by the replicase encoded by the first RNA molecule, optionally wherein the first or second RNA molecule, preferably the second RNA molecule, further comprises at least one open reading frame (ORF) encoding a protein of interest.

123. The first RNA molecule and second RNA molecule for use according to claim 122, wherein the therapy is the treatment or prevention of cancer or an infectious disease.
